(19) United States
(12) Patent Application Publication
(10) Pub. No.: US 2012/0101091 A1
(43) Pub. Date: Apr. 26, 2012

(54) VIRAL POLYMERASE INHIBITORS

(75) Inventors: Pierre Beaulieu, Rosemère (CA); Pierre Bonneau, Blainville (CA); René Coulombe, Montréal (CA); Pasquale Forgione, Montréal (CA); James Gillard, Rosemère (CA); Araz Jakalian, Laval (CA); Jean Rancourt, Laval (CA)

(73) Assignee: BOEHRINGER INGELHEIM INTERNATIONAL GMBH, Ingelheim am Rhein (DE)

(21) Appl. No.: 13/122,211

(22) PCT Filed: Oct. 1, 2009
(86) PCT No.: PCT/CA2009/001346

§ 371 Date: Sep. 7, 2011

Related U.S. Application Data
(60) Provisional application No. 61/102,593, filed on Oct. 3, 2008.

(51) Int. Cl. A61K 31/5377; C07D 407/10; C07D 417/12; C07D 413/10; C07D 405/10; C07D 405/12; C07D 413/14; A61K 31/343; A61K 31/427; A61K 31/443; A61K 31/4245; A61K 31/351; A61K 31/4196; A61P 31/14; C07D 307/91

(52) U.S. Cl. 514/232.8; 549/461; 548/200; 544/153; 546/284.1; 548/131; 549/414; 548/264.2; 544/131; 514/468; 514/365; 514/337; 514/364; 514/459; 514/384

(57) ABSTRACT

The present application provides compounds of formula I wherein X, Y, R², n, R⁵ and R⁶ are defined herein, useful as inhibitors of the hepatitis C virus NS5B polymerase The present application also provides pharmaceutical compositions containing said compounds, methods of using said compounds as pharmaceuticals alone or with other antiviral agent in the treatment of a hepatitis C viral infection in a mammal having or at risk of having the infection.

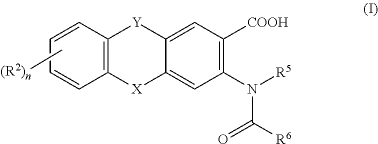

### RELATED APPLICATION

This application claims benefit of U.S. Ser. No. 61/102,593 filed Oct. 3, 3008, which is herein incorporated by reference.

### FIELD OF THE INVENTION

The present invention relates to compounds, compositions and methods for the treatment of hepatitis C virus (HCV) infection. In particular, the present invention provides novel inhibitors of the hepatitis C virus NS5B polymerase, pharmaceutical compositions containing such compounds and methods for using these compounds in the treatment of HCV infection.

### BACKGROUND OF THE INVENTION

it is estimated that at least 170 million persons worldwide are infected with the hepatitis C virus (HCV). Acute HCV infection progresses to chronic infection in a high number of cases, and, in some infected individuals, chronic infection leads to serious liver diseases such as cirrhosis and hepatocellular carcinoma.

Currently, standard treatment of chronic hepatitis C infection involves administration of pegylated interferon-alpha in combination with ribavirin. However, this therapy is not effective in reducing HCV RNA to undetectable levels in many infected patients and is associated with often intolerable side effects such as fever and other influenza-like symptoms, depression, thrombocytopenia and hemolytic anemia. Furthermore, some HCV-infected patients have co-existing conditions which contraindicate this treatment.

Therefore, a need exists for alternative treatments for hepatitis C viral infection. One possible strategy to address this need is the development of effective antiviral agents which inactivate viral or host cell factors which are essential for viral replication.

HCV is an enveloped positive strand RNA virus in the genus Hepacivirus in the Flaviviridae family. The single strand HCV RNA genome is approximately 9500 nucleotides in length and has a single open reading frame (ORF), flanked by 5′ and 3′ non-translated regions. The HCV 5′ non-translated region is 341 nucleotides in length and functions as an internal ribosome entry site for cap-independent translation initiation. The open reading frame encodes a single large polyprotein of about 3000 amino acids which is cleaved at multiple sites by cellular and viral proteases to produce the mature structural and non-structural (NS2, NS3, NS4A, NS4B, NS5A, and NS5B) proteins. The viral NS2/3 protease cleaves at the NS2-NS3 junction; while the viral NS3 protease mediates the cleavages downstream of NS3, at the NS3-NS4A, NS4A-NS4B, NS4B-NS5A and NS5A-NS5B cleavage sites. The NS3 protein also exhibits nucleoside triphosphatase and RNA helicase activities. The NS4A protein acts as a cofactor for the NS3 protease and may also assist in the membrane localization of NS3 and other viral replicase components. Although NS4B and the NS5A phosphoprotein are also likely components of the replicase, their specific roles are unknown. The NS5B protein is the elongation subunit of the HCV replicase possessing RNA-dependent RNA polymerase (RdRp) activity.

The development of new and specific anti-HCV treatments is a high priority, and virus-specific functions essential for replication are the most attractive targets for drug development. The absence of RNA dependent RNA polymerases in non-human mammals, and the fact that this enzyme appears to be essential to viral replication, would suggest that the NS5B polymerase is an ideal target for anti-HCV therapeutics. It has been recently demonstrated that mutations destroying NS5B activity abolish infectivity of RNA in a chimp model (Kolykhalov, A. A.; Mihalik K.; Feinstone, S. M.; Rice, C. M.; 2000; J. Viral. 74, 2046-2051).

WO 2007/087717 and WO 2008/0019477 disclose compounds of the general formula (A):

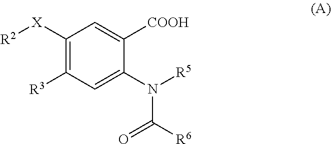

wherein R² is an optionally substituted aryl or Het which are useful for the treatment of Hepatitis C virus infections.

### SUMMARY OF THE INVENTION

The present invention provides a novel series of compounds having inhibitory activity against HCV polymerase. In particular compounds according to this invention inhibit RNA synthesis by the RNA dependent RNA polymerase of HCV, especially the enzyme NS5B encoded by HCV. A further advantage of compounds provided by this invention is their low to very low or even non-significant activity against other polymerases. Further objects of this invention arise for the one skilled in the art from the following description and the examples.

One aspect of the invention provides compounds of formula (I):

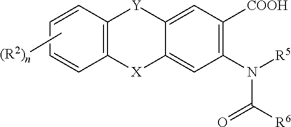

wherein:
either X is absent and Y is O; or
   Y is absent and X is O;
n is 0 to 4;
R² is selected from:
   a) halo, cyano, nitro or SO₃H;
   b) R⁷, —C(═O)—R⁷, —C(═O)—O—R⁷, —O—R⁷, —S—R⁷, —SO—R⁷, —SO₂—R⁷, —(C₁₋₆)alkylene-R⁷, —(C₁₋₆)alkylene-C(═O)—R⁷, —(C₁₋₆)alkylene-C(═O)—O—R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl; (C₃₋₇)cycloalkyl, —(C₁₋₆)alkyl-(C₃₋₇)cycloalkyl, aryl and Het;
      wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl, —N((C₁₋₄))alkyl)₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
      wherein each of the aryl and Het is optionally substituted with 1 to 3 substituents each independently selected from:
      i) halo, cyano, oxo, thioxo, imino, —OH, —O—(C₁₋₆)alkyl, —O—(C₁₋₆)haloalkyl, —(C₃₋₇)cycloalkyl, —(C₁₋₆)haloalkyl, —C(═O)—(C₁₋₆)alkyl, COOH, —SO₂(C₁₋₆)alkyl, —C(═O)—NH₂, —C(═O)—NH(C₁₋₄)alkyl; —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —O—C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹, —(C₁₋₆)alkylene-O—C(═O)—N(R⁸)R⁹, or —(C₁₋₆)alkylene-SO₂—N(R⁸)R⁹; wherein the (C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C)₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
      R⁸ is in each instance independently selected from H, (C₁₋₆)alkyl and (C₃₋₇)cycloalkyl; and
      R⁹ is in each instance independently selected from R⁷, —O—(C₁₋₆)alkyl, —(C₁₋₆)alkylene-R⁷, —SO₂—R⁷, —C(═O)—R⁷, —C(═O)OR⁷ and —C(═O)N(R⁸)R⁷, wherein R⁷ and R⁸ are as defined above;
         or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
         wherein the heterocycle is optionally substituted with 1 to 3 substituents each independently selected from (C₁₋₆)alkyl, (C₁₋₆)haloalkyl, halo, oxo, —OH, —SH, —O(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
R⁵ is H, (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het; each being optionally substituted with 1 to 4 substituents each independently selected from (C₁₋₆)alkyl, (C₁₋₆)haloalkyl, (C₃₋₇)cycloalkyl, Het, —OH, —COOH, —C(═O)—(C₁₋₆)alkyl, —C(═O)—O—(C₁₋₆)alkyl, —SO₂(C₁₋₆)alkyl, —C(═O)—N(R⁵¹)R⁵² and —O—R⁵³; wherein R⁵³ is C₁₋₆)alkyl, —(C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het, said aryl and Het being optionally substituted with (C₁₋₆)alkyl or —O—(C₁₋₆)alkyl;
   wherein R⁵¹ is H, (C₁₋₆)alkyl or (C₃₋₇)cycloalkyl; and
   R⁵² is H, (C₁₋₆)alkyl, (C₃₋₇)cycloalkyl, aryl, Het, —(C₁₋₃)alkyl-aryl or —(C₁₋₃)alkyl-Het;
      wherein each of the (C₁₋₆)alkyl, (C₃₋₇)cycloalkyl, aryl, Het, —(C₁₋₃)alkyl-aryl and —(C₁₋₃)alkyl-Het are optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N(C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
         wherein the (C₁₋₆)alkyl is optionally substituted with OH;
   or R⁵¹ and R⁵², together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
      wherein the heterocycle is optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
R⁶ is (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het; being optionally substituted with 1 to 5 substituents each independently selected from halo, (C₁₋₆)alkyl, (C₁₋₆)haloalkyl, (C₃₋₇)cycloalkyl; —OH, —SH, —O—(C₁₋₄)alkyl, —S—(C₁₋₄)alkyl and —N(R⁸)R⁹, wherein R⁸ and R⁹ are as defined above; and
Het is a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S, wherein each N heteroatom may, independently and where possible, exist in an oxidized state such that it is further bonded to an oxygen is atom to form an N-oxide group and wherein each S heteroatom may independently and where possible, exist in an oxidized state such that it is further bonded to one or two oxygen atoms to form the groups SO or SO₂;
or a salt or ester thereof.

Another aspect of this invention provides a compound of formula (I), or a pharmaceutically acceptable salt or ester thereof, as a medicament.

Still another aspect of this invention provides a pharmaceutical composition comprising a therapeutically effective amount of a compound of formula (I) or a pharmaceutically acceptable salt or ester thereof; and one or more pharmaceutically acceptable carriers.

According to an embodiment of this aspect, the pharmaceutical composition according to this invention additionally comprises at least one other antiviral agent.

The invention also provides the use of a pharmaceutical composition as described hereinabove for the treatment of a hepatitis C viral infection in a mammal having or at risk of having the infection.

A further aspect of the invention involves a method of treating a hepatitis C viral infection in a mammal having or at risk of having the infection, the method comprising administering to the mammal a therapeutically effective amount of a compound of formula (I), a pharmaceutically acceptable salt or ester thereof, or a composition thereof as described hereinabove.

Another aspect of the invention involves a method of treating a hepatitis C viral infection in a mammal having or at risk of having the infection, the method comprising administering to the mammal a therapeutically effective amount of a combination of a compound of formula (I) or a pharmaceutically acceptable salt or ester thereof, and at least one other antiviral agent; or a composition thereof.

Also within the scope of this invention is the use of a compound of formula (I) as described herein, or a pharmaceutically acceptable salt or ester thereof, for the treatment of a hepatitis C viral infection in a mammal having or at risk of having the infection.

Another aspect of this invention provides the use of a compound of formula (I) as described herein, or a pharmaceutically acceptable salt or ester thereof, for the manufacture of a medicament for the treatment of a hepatitis C viral infection in a mammal having, or at risk of having the infection.

An additional aspect of this invention refers to an article of manufacture comprising a composition effective to treat a hepatitis C viral infection; and packaging material comprising a label which indicates that the composition can be used to treat infection by the hepatitis C virus; wherein the composition comprises a compound of formula (I) according to this invention or a pharmaceutically acceptable salt or ester thereof.

Still another aspect of this invention relates to a method of inhibiting the replication of hepatitis C virus comprising exposing the virus to an effective amount of the compound of formula (I), or a salt or ester thereof, under conditions where replication of hepatitis C virus is inhibited.

Further included in the scope of the invention is the use of a compound of formula (I), or a salt or ester thereof, to inhibit the replication of hepatitis C virus.

### DETAILED DESCRIPTION OF THE INVENTION

### Definitions

As used herein, the following definitions apply unless otherwise noted:

The term “substituent”, as used herein and unless specified otherwise, is intended to mean an atom, radical or group which may be bonded to a carbon, atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The term “(C₁₋ₙ)alkyl” as used herein, wherein n is an integer, either alone or in combination with another radical, is intended to mean acyclic, straight or branched chain alkyl radicals containing from 1 to n carbon atoms and includes, but is not limited to, methyl, ethyl, propyl (n-propyl), butyl (n-butyl), 1-methylethyl (iso-propyl), 1-methylpropyl (sec-butyl), 2-methylpropyl (iso-butyl), 1,1-dimethylethyl (tert-butyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

The term “(C₁₋ₙ)alkylene” as used herein, wherein n is an integer, either alone or in combination with another radical, is intended to mean acyclic, straight or branched chain divalent alkyl radicals containing from 1 to n carbon atoms and includes, but is not limited to —CH₂—, —CH₂CH₂—,

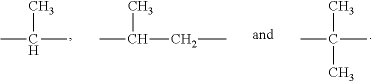

The term “(C₂₋ₙ)alkenyl”, as used herein, wherein n is an integer, either alone or in combination with another radical, is intended to mean an unsaturated, acyclic straight or branched chain radical containing two to n carbon atoms, at least two of which are bonded to each other by a double bond. Examples of such radicals include, but are not limited to, ethenyl (vinyl), 1-propenyl, 2-propenyl, and 1-butenyl. Unless specified otherwise, the term “(C₂₋ₙ)alkenyl” is understood to encompass individual stereoisomers where possible, including but not limited to (E) and (Z) isomers, and mixtures thereof. When a (C₂₋ₙ) alkenyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “(C₂₋ₙ)alkynyl”, as used herein, wherein n is an integer, either alone or in combination with another radical, is intended to mean an unsaturated, acyclic straight or branched chain radical containing two to n carbon atoms, at least two of which are bonded to each other by a triple bond. Examples of such radicals include, but are not limited to ethynyl, 1-propynyl, 2-propynyl, and 1-butynyl. When a (C₂₋ₙ)alkynyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “(C₃₋ₘ)cycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl and cycloheptyl. The term “—(C₁₋ₙ)alkyl-(C₃₋ₘ)cycloalkyl” as used herein, wherein n and m are both integers, either alone or in combination with another radical, is intended to mean an alkyl radical having 1 to n carbon atoms as defined above which is itself substituted with a cycloalkyl radical containing from 3 to m carbon atoms as defined above, and includes, but is not limited to, cyclopropylmethyl, cyclobutylmethyl, cyclopentylmethyl, cyclohexylmethyl, 1-cyclopropylethyl, 2-cyclopropylethyl, 1-cyclobutylethyl, 2-cyclobutylethyl, 1-cyclopentylethyl, 2-cyclopentylethyl, 1-cyclohexylethyl and 2-cyclohexylethyl. When a (C₃₋ₘ)-cycloalkyl-(C₁₋ₙ)alkyl- group is substituted, it is understood that substituents may be attached to either the cycloalkyl or the alkyl portion thereof or both, unless specified otherwise.

The term “aryl” as used herein, either alone or in combination with another radical, is intended to mean a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, indenyl, 1-naphthyl, 2-naphthyl, tetrahydronaphthyl and dihydronaphthyl.

The term “—(C₁₋ₙ)alkyl-aryl” as used herein, wherein n is an integer, either alone or in combination with another radical, is intended to mean an alkyl radical having 1 to n carbon atoms as defined above which is itself substituted with an aryl radical as defined above. Examples of aryl-(C₁₋ₙ)alkyl- include, but are not limited to, phenylmethyl (benzyl), 1-phenylethyl, 2-phenylethyl and phenylpropyl. When an aryl-(C₁₋ₙ)alkyl- group is substituted, it is understood that substituents may be attached to either the aryl or the alkyl portion thereof or both, unless specified otherwise.

The term “Het” as used herein, either alone or in combination with another radical, is intended to mean a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S; wherein each N heteroatom may, independently and where possible, exist in an oxidized state such that it is further bonded to an oxygen atom to form an N-oxide group and wherein each S heteroatom may, independently and where possible, exist in an oxidized state such that it is further bonded to one or two oxygen atoms to form the groups SO or SO₂, unless specified otherwise. When a Het group is substituted, it is understood that substituents may be attached to any carbon atom or heteroatom thereof which would otherwise bear a hydrogen atom, unless specified otherwise.

The term “—(C₁₋ₙ)alkyl-Het” as used herein and unless specified otherwise, wherein n is an integer, either alone or in combination with another radical, is intended to mean an alkyl radical having 1 to n carbon atoms as defined above which is itself substituted with a Het substituent as defined above. Examples of Het-(C₁₋ₙ)alkyl-include, but are not limited to, thienylmethyl, furylmethyl, piperidinylethyl, 2-pyridinylmethyl, 3-pyridinylmethyl, 4-pyridinylmethyl, quinolinylpropyl, and the like. When a Het-(C₁₋ₙ)alkyl- group is substituted, it is understood that substituents may be attached to either the Het or the alkyl portion thereof or both, unless specified otherwise.

The term “heteroatom” as used herein is intended to mean O, S or N.

The term “heterocycle” as used herein and unless specified otherwise, either alone or in combination with another radical, is intended to mean a 4- to 7-membered saturated, unsaturated or aromatic heterocycle containing from 1 to 4 heteroatoms each independently selected from O, N and S; or a monovalent radical derived by removal of a hydrogen atom therefrom. Examples of such heterocycles include, but are not limited to, azetidine, pyrrolidine, tetrahydrofuran, tetrahydrothiophene, thiazolidine, oxazolidine, pyrrole, thiophene, furan, pyrazole, imidazole, isoxazole, oxazole, isothiazole, thiazole, triazole, tetrazole, piperidine, piperazine, azepine, diazepine, pyran, 1,4-dioxane, 4-morpholine, 4-thiomorpholine, pyridine, pyridine-N-oxide, pyridazine, pyrazine, pyrimidine, and the following heterocycles:

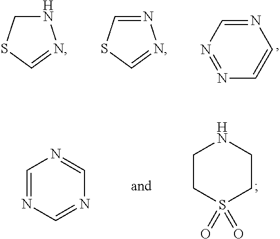

and saturated, unsaturated and aromatic derivatives thereof.

The term “heteropolycycle” as used herein and unless specified otherwise, either alone or in combination with another radical, is intended to mean a heterocycle as defined above fused to one or more other cycle, including a carbocycle, a heterocycle or any other cycle; or a monovalent radical derived by removal of a hydrogen atom therefrom. Examples of such heteropolycycles include, but are not limited to, indole, isoindole, benzimidazole, benzothiophene, benzofuran, benzodioxole, benzothiazole, quinoline, isoquinoline, naphthyridine, and the following heteropolycycles:

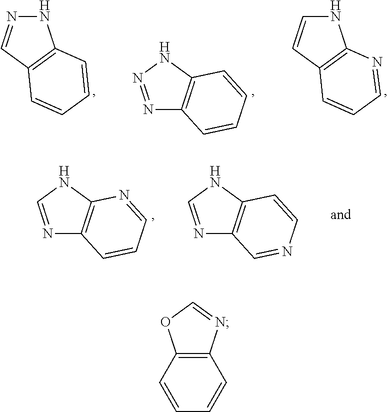

and saturated, unsaturated and aromatic derivatives thereof.

The term “halo” as used herein is intended to mean a halogen substituent selected from fluoro, chloro, bromo or iodo.

The term “(C₁₋ₙ)haloalkyl” as used herein, wherein n is an integer, either alone or in combination with another radical, is intended to mean an alkyl radical having 1 to n carbon atoms as defined above wherein one or more hydrogen atoms are each replaced by a halo substituent. Examples of (C₁₋ₙ)haloalkyl include but are not limited to chloromethyl, chloroethyl, dichloroethyl, bromomethyl, bromoethyl, dibromoethyl, fluoromethyl, difluoromethyl, trifluoromethyl, fluoroethyl and difluoroethyl.

The terms “—O—(C₁₋ₙ)alkyl” or “(C₁₋ₙ)alkoxy” as used herein interchangeably, wherein n is an integer, either alone or in combination with another radical, is intended to mean an oxygen atom further bonded to an alkyl radical having 1 to n carbon atoms as defined above. Examples of −O—(C₁₋ₙ)alkyl include but are not limited to methoxy (CH₃O—), ethoxy (CH₃CH₂O—), propoxy (CH₃CH₂CH₂O—), 1-methylethoxy (iso-propoxy; (CH₃)₂CH—O—) and 1,1-dimethylethoxy (tert-butoxy; (CH₃)₃C—O—). When an —O—(C₁₋ₙ)alkyl radical is substituted, it is understood to be substituted on the (C₁₋ₙ)alkyl portion thereof.

The terms “—S—(C₁₋ₙ)alkyl” or “(C₁₋ₙ)alkylthio” as used herein interchangeably, wherein n is an integer, either alone or in combination with another radical, is intended to mean an sulfur atom further bonded to an alkyl radical having 1 to n carton atoms as defined above. Examples of —S—(C₁₋ₙ)alkyl include but are not limited to methylthio (CH₃S—), ethylthio (CH₃CH₂S—), propylthio (CH₃CH₂CH₂S—), 1-methylethylthio (isopropylthio; (CH₃)₂CH—S—) and 1,1-dimethylethylthio (tert-butylthio; (CH₃)₃C—S—). When —S—(C₁₋ₙ)alkyl radical, or an oxidized derivative thereof, such as an —SO—(C₁₋ₙ)alkyl radical or an —SO₂—(C₁₋ₙ)alkyl radical, is substituted, each is understood to be substituted on the (C₁₋ₙ)alkyl portion thereof.

The term “oxo” as used herein is intended to mean an oxygen atom attached to a carbon atom as a substituent by a double bond (═O).

The term “thioxo” as used herein is intended to mean a sulfur atom attached to a carbon atom as a substituent by a double bond (═S).

The term “imino” as used herein is intended to mean a NH group attached to carbon atom as a substituent by a double bond (═NH).

The term “cyano” or “CN” as used herein is intended to mean a nitrogen atom attached to a carbon atom by a triple bond (C≡N).

The term “COOH” as used herein is intended to mean a carboxyl group (—C(═O)—OH). It is well known to one skilled in the art that carboxyl groups may be substituted by functional group equivalents. Examples of such functional group equivalents contemplated in this invention include, but are not limited to, esters, amides, imides, boronic acids, phosphonic acids, phosphoric acids, tetrazoles, triazoles, N-acylsulfamides (RCONHSO₂NR₂), and N-acylsulfonamides (RCONHSO₂R).

The term “functional group equivalent” as used herein is intended to mean an atom or group that may replace another atom or group which has similar electronic, hybridization or bonding properties.

The term “protecting group” as used herein is intended to mean protecting groups that can be used during synthetic transformation, including but not limited to examples which are listed in Greene, “Protective Groups in Organic Chemistry”, John Wiley & Sons, New York (1981), and more recent editions thereof, herein incorporated by reference.

The following designation [symbol] is used in sub-formulas to indicate the bond which is connected to the rest of the molecule as defined.

The term “salt thereof” as used herein is intended to mean any acid and/or base addition salt of a compound according to the invention, including but not limited to a pharmaceutically acceptable salt thereof.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Berge et al., J. Pharm. Sci. 1977, 66, pp. 1-19, herein incorporated by reference.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, sulfuric acid, sulfamic acid, nitric acid, phosphoric acid and the like, and organic acids including but not limited to acetic acid, trifluoroacetic acid, adipic ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like. Particularly preferred are the ammonium, potassium, sodium, calcium, and magnesium salts. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins and the like. Particularly preferred organic nontoxic bases are isopropylamine, diethylamine, ethanolamine, trimethylamine, dicyclohexylamine, choline, and caffeine.

The term “ester thereof” as used herein is intended to mean any ester of a compound according to the invention in which any of the —COOH substituents of the molecule is replaced by a —COOR substituent, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl, each of which being optionally further substituted. The term “ester thereof” includes but is not limited to pharmaceutically acceptable esters thereof.

The term “pharmaceutically acceptable ester” as used herein is intended to mean esters of the compound according to the invention in which any of the COOH substituents of the molecule are replaced by a —COOR substituent, in which the R moiety of the ester is selected from alkyl (including, but not limited to, methyl, ethyl, propyl, 1-methylethyl, 1,1-dimethylethyl, butyl); alkoxyalkyl (including, but not limited to methoxymethyl), acyloxyalkyl (including, but not limited to acetoxymethyl); arylalkyl (including, but not limited to, benzyl); aryloxyalkyl (including, but not limited to, phenoxymethyl); and aryl (including, but not limited to phenyl) optionally substituted with halogen, (C₁₋₄)alkyl or (C₁₋₄)alkoxy. Other suitable esters can be found in Design of Prodrugs. Bundgaard, H. Ed Elsevier (1985), herein incorporated by reference. Such pharmaceutically acceptable esters are usually hydrolyzed in vivo when injected into a mammal and transformed into the acid form of the compound according to the invention. With regard to the esters described above, unless otherwise specified, any alkyl moiety present preferably contains 1 to 16 carbon atoms, more preferably 1 to 6 carbon atoms. Any aryl moiety present in such esters preferably comprises a phenyl group. In particular the esters may be a (C₁₋₁₆)alkyl ester, an unsubstituted benzyl ester or a benzyl ester substituted with at least one halogen, (C₁₋₆)alkyl, (C₁₋₆)alkoxy, nitro or trifluoromethyl.

The term “mammal” as used herein is intended to encompass humans, as well as non-human mammals which are susceptible to infection by hepatitis C virus. Non-human mammals include but are not limited to domestic animals, such as cows, pigs, horses, dogs, cats, rabbits, rats and mice, and non-domestic animals.

The term “treatment” as used herein is intended to mean the administration of a compound or composition according to the present invention to alleviate or eliminate symptoms of the hepatitis C disease and/or to reduce viral load in a patient. The term “treatment” also encompasses the administration of a compound or composition according to the present invention post-exposure of the individual to the virus but before the appearance of symptoms of the disease, and/or prior to the detection of the virus in the blood, to prevent the appearance of symptoms of the disease and/or to prevent the virus from reaching detectable levels in the blood.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal.

The term “Therapeutically effective amount” means an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

### Preferred Embodiments

In the following preferred embodiments, groups and substituents of the compounds of formula (I):

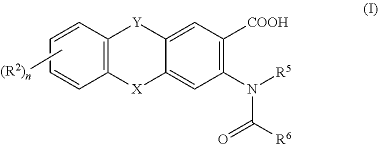

are described in detail,

### Core:

Core-A: In one embodiment, the Core is:

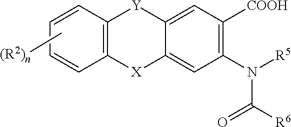

wherein R², n, R⁵ and R⁶ are as defined herein; and
wherein X and Y are defined as:
X, Y-A: In one embodiment, X is O and Y is absent,
X, Y-B: In one embodiment, Y is O and X is absent.
Any and each individual definition of X, Y as set out herein may be combined with any and each individual definition of n, R², R⁵ and R⁶ as set out herein.
Core-B: In another embodiment, the Core is:

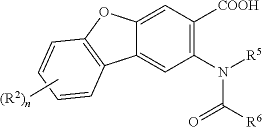

wherein R², n, R⁵ and R⁶ are as defined herein.
Core-C: In another embodiment, the Core is:

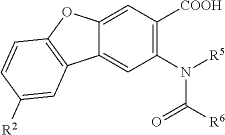

wherein R², R⁵ and R⁶ are as defined herein.
Core-D: In another embodiment, the Core is:

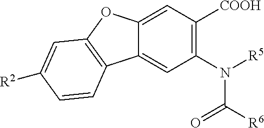

wherein R², R⁵ and R⁶ are as defined herein.
Core-E: In another embodiment, the Core:

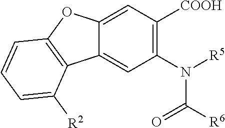

wherein R², R⁵ and R⁶ are as defined herein.
Core-F: In another embodiment, the Core is:

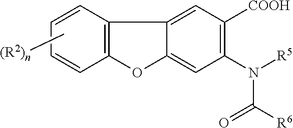

wherein R², n, R⁵ and R⁶ are as defined herein.
Core-G: In another embodiment, the Core is:

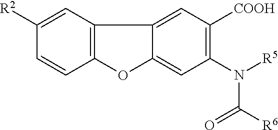

wherein R², R⁵ and R⁶ are as defined herein.
Core-H: In one embodiment, the Core is:

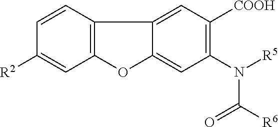

wherein R², R⁵ and R⁶ are as defined herein.
Core-I: In one embodiment, the Core is:

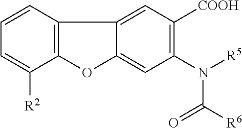

wherein R², R⁵ and R⁶ are as defined herein.

Any and each individual definition of the Core as set out herein may be combined with any and each individual definition of n, R², R⁵ and R⁶ as set out herein.

### R²:

R²-A: In one embodiment, R² is selected from:
   a) halo, cyano, nitro or SO₃H;
   b) R⁷, —C(═O)—R⁷, —C(═O)—O—R⁷, —O—R⁷, —S—R⁷, —SO—R⁷, —SO₂—R⁷, —(C₁₋₆)alkylene-R⁷, —(C₁₋₆)alkylene-C(═O)—R⁷, —(C₁₋₆)alkylene-C(═O)—O—R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalky, aryl and Het;
      wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl, —N((C₁₋₄)alkyl)₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
      wherein each of the aryl and Het is optionally substituted with 1 to 3 substituents each independently selected from:
      i) halo, cyano, oxo, thioxo, amino, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —O—C(═O)—N(R⁸)R⁹, —SO₂N(R⁸)R⁹, —C₁₋₆)alkylene-N(R⁸)R⁹, —(C₁₋₆)alkylene-C(═O)—N(R⁸)R⁹, —(C₁₋₆)alkylene-O—C(═O)—N(R⁸)R⁹, or —(C₁₋₆)alkylene-SO₂—N(R⁸)R⁹; wherein the (C₁₋₆)alkylene is optionally substituted with 1 or 2 substituents each independently selected from —OH, —(C₁₋₆)alkyl, halo, (C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N(C₁₋₄)alkyl)₂;
      R⁸ is in each instance independently selected from H, (C₁₋₆)alkyl and (C₃₋₇)cycloalkyl and
      R⁹ is in each instance independently selected from R⁷, —O—(C₁₋₆)alkyl, —(C₁₋₆)alkylene-R⁷, —SO₂—R⁷, —C(═O)—R⁷, —C(═O)OR⁷ and —C(═O)N(R⁸)R⁷; wherein R⁷ and R⁸ are as defined, above;
         or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
         wherein the heterocycle is optionally substituted with 1 to 3 substituents each independently selected from (C₁₋₆)alkyl, (C₁₋₆)haloalkyl, halo, oxo, —OH, —SH, —O(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl.
R²-B: In another embodiment, R² is selected from;
   a) halo, nitro or SO₃H;
   b) R⁷, —C(═O)—R⁷, —O—R⁷, —SO—R⁷, —SO₂—R⁷, —(C₁₋₆)alkylene-R⁷, —(C₁₋₆)alkylene-C(═O)—R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
      wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₁₋₄)alkyl, —NH(C₃₋₇)cycloalkyl, —N((C₁₋₄)alkyl)(C₃₋₇)cycloalkyl, —N((C₁₋₄)alkyl)₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
      wherein each of the aryl and Het is optionally substituted, with 1 to 3 substituents each independently selected from:
      i) halo, cyano, oxo, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —O—C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹, —(C₁₋₆)alkylene-O—C(═O)—N(R⁸)R⁹, or —(C₁₋₆)alkylene-SO₂—N(R⁸)R⁹; wherein the (C₁₋₆)alkylene is optionally substituted with 1 or 2 substituents each independently selected from —OH, —(C₁₋₆)alkyl, halo, (C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
      R⁸ is in each instance independently selected from H, (C₁₋₆)alkyl and (C₃₋₇)cycloalkyl; and
      R⁹ is in each instance independently seeded from R⁷, —O—(C₁₋₆)alkyl, —(C₁₋₆)alkylene-R⁷, —SO₂—R⁷, —C(═O)—R⁷, —C(═O)OR⁷ and —C(═O)N(R⁸)R⁷; wherein R⁷ and R⁸ are as defined above.
R²-C: In another embodiment, R² is selected from:
   a) halo, nitro or SO₃H;
   b) R⁷, —C(═O)—R⁷, —O—R⁷, —S—R⁷, —SO—R⁷, —SO₂—R⁷, —(C₁₋₆)alkylene-R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
      wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, —(C₁₋₆)haloalkyl, (C₃₋₇)cycloalkyl, —O—(C₁₋₆)alkyl, cyano, COOH, —NH₂, —NH₂, —NH(C₁₋₄)alkyl, —NH(C₃₋₇)cycloalkyl, —N((C₁₋₄)alkyl)(C₃₋₇)cycloalkyl, —N((C₁₋₄)alkyl₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
      wherein each of the aryl and Het is optionally substitute with 1 to 3 substituents each independently selected from:
      i) halo, cyano, oxo, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N(((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —SO₂—N(R^{B})R⁹, —(C₁₋₆)alkylene-N(R⁸)R⁹, —(C₁₋₆)alkylene-C(═O)—N(R⁸)R⁹ or —(C₁₋₆)alkylene-SO₂—N(R⁹)R⁹; wherein the (C₁₋₆)alkylene is optionally substituted with 1 or 2 substituents each independently selected from —OH, —(C₁₋₆)alkyl, halo, (C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
      R⁸ is in each instance independently selected from H and (C₁₋₆)alkyl; and
      R⁹ is in each instance independently selected from R⁷, —O—(C₁₋₆)alkyl, —(C₁₋₆)alkylene-R⁷, —SO₂—R⁷, —C(═O)—R⁷; wherein R⁷ and R⁸ are as defined above.
R²-D: In another embodiment, R² is selected from:
   a) halo, nitro or SO₃H;
   b) R⁷, C(═O)OH, C(═O)(C₁₋₆)alkyl, —O—R⁷, —S—R⁷, —SO—R⁷, —SO₂—R⁷, —(C₁₋₆)alkylene-R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
      wherein the (C₁₋₆)alkyl, (C₂₋₆)alkenyl, (C₁₋₆)alkynyl, (C₁₋₆)haloalkyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl, —N((C₁₋₄)alkyl₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
      wherein each of the aryl and Het is optionally substituted with 1 to 3 substituents each independently selected from:
      i) halo, cyano, oxo, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹ or —(C₁₋₆)alkylene-SO₂—N(R⁸)R⁹; wherein the (C₁₋₆)alkylene is optionally substituted with 1 or 2 substituents each independently selected from —OH, —(C₁₋₆)alkyl, halo, (C₁₋₆)haloalkyl, (C₃₋₇)cycloalkyl, —O—(C₁₋₆)alkyl, cyano, COOH, —NH₂, —NH₂, —NH(C₁₋₄)alkyl and —N((C₁₋₄)alkyl)₂;
      R⁸ is in each instance independently selected from H and (C₁₋₆)alkyl; and
      R⁹ is in each instance independently selected from R⁷, —O—(C₁₋₆)alkyl; —(C₁₋₆)alkylene-R⁷, —SO₂—R⁷, —C(═O)—R⁷; wherein R⁷ is as defined above,
R²-E: In another embodiment, R² is selected from:
   a) halo, nitro or SO₃H;
   b) R⁷, C(═O)OH, C(═O)(C₁₋₆)alkyl, —O—R⁷, —SO₂—R⁷, —(C₁₋₆)alkylene-R⁷, —(C₁₋₆)alkylene-O—R⁷, —(C₁₋₆)alkylene-S—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
      wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, —(C₁₋₆)haloalkyl, (C₃₋₇)cycloalkyl, —O—(C₁₋₆)alkyl, COOH, —NH₂, —N((C₁₋₄)alkyl)(aryl), aryl, —(C₁₋₆)alkyl-aryl, aryl, —S—(C₁₋₆)alkyl-aryl, Het, —(C₁₋₆)alkyl-Het, —O—(C₁₋₆)alkyl-Het; and
      wherein each of the aryl and Het is optionally substituted with 1 to 3 substituents each independently selected from:
      i) halo, cyano, oxo, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —C(═O)—NH₂, —C(═O)—NH(C₁₋₄)alkyl, —C(═O)—N((C₁₋₄)alkyl)₂, —NH₂, —NH(C₁₋₄)alkyl, —N(C₁₋₄)alkyl)₂ or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹ or —(C₁₋₆)alkylene-SO₂—N(R⁸)R⁹; wherein the (C₁₋₆)alkylene is optionally substituted with 1 or 2 substituents each independently selected from —OH, —(C₁₋₆)alkyl, halo, —(C₁₋₆)haloalkyl, —O—(C₁₋₆)alkyl;
      R⁸ is in each instance independently selected from H and (C₁₋₆)alkyl; and
      R⁹ is in each instance independently selected from R⁷, —O—(C₁₋₆)alkyl, —(C₁₋₆)alkylene-R⁷, —C(═O)—R⁷; wherein R⁷ is as defined above.
R²-F: In another embodiment, R² is selected from:
   a) halo, nitro or SO₃H;
   b) R⁷, OH, C(═O)OH, C(═O)(C₁₋₆)alkyl, —SO₂—R⁷, —(C₁₋₆)alkylene-R⁷, —(C₁₋₆)alkylene-O—R⁷, —(C₁₋₆)alkylene-S—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
      wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, —(C₁₋₆)haloalkyl, (C₃₋₇)cycloalkyl, O—(C₁₋₆)alkyl, COOH, —NH₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, O—(C₁₋₆)alkyl-Het; and
      wherein each of the aryl and Het is optionally substituted with 1 to 3 substituents each independently selected from:
      i) halo, cyano, oxo, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —C(═O)NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂ or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —SO₂—N(R⁸)R⁹, —(C₁₋₆)alkylene-N(R⁸)R⁹ or —(C₁₋₆)alkylene-C(═O)—N(R⁸)R⁹; wherein the (C₁₋₆)alkylene is optionally substituted with 1 or 2 substituents each independently selected from —OH, —(C₁₋₆)alkyl, halo, —(C₁₋₆)haloalkyl, —O—(C₁₋₆)alkyl;
      R⁸ is in each instance independently selected from H and (C₁₋₆)alkyl; and
      R⁹ is in each instance independently selected from R⁷, —O—(C₁₋₆)alkyl, —(C₁₋₆)alkylene-R⁷, —C(═O)—R⁷; wherein R⁷ is as defined above.
R²-G: In another embodiment, R² is selected from:
   a) halo, nitro or SO₃H;
   b) R⁷, OH, C(═O)OH, C(═O)(C₁₋₆)alkyl, —SO₂—R⁷, —(C₁₋₆)alkylene-R⁷, —(C₁₋₆)alkylene-O—R⁷, —(C₁₋₆)alkylene-S—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
      wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, halo, (C₁₋₆)haloalkyl, —O—(C₁₋₆)alkyl, COOH, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
      wherein each of the aryl and Het is optionally substituted with 1 to 3 substituents each independently selected from:
      i) halo, cyano, oxo, —OH, —O—(C₁₋₆)alkyl, (C₁₋₆)haloalkyl, —NH₂, —N((C₁₋₄)alkyl)₂ or —NH—C(═O)(C₁₋₄)alkyl;
      ii) (C₁₋₆)alkyl optionally substituted with —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —SO₂—N(R⁸)R⁹ or —(C₁₋₆)alkylene-N(R⁸)R⁹; R⁸ is H; and
      R⁹ is in each instance independently selected from R⁷, —(C₁₋₆)alkylene-R⁷ or —C(═O)—R⁷, wherein R⁷ is as defined above.
R²-H: In another embodiment, R² is selected from:
   a) halo, nitro or SO₃H;
   b) R⁷, OH, C(═O)OH, C(═O)(C₁₋₆)alkyl, —SO₂—R⁷, —(C₁₋₆)alkylene-R⁷, —(C₁₋₆)alkylene-O—R⁷, —(C₁₋₆)alkylene-S—R⁷ or —(C₁₋₆)alkylene-SO₂—R⁷;
      wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
      wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, halo, (C₁₋₆)haloalkyl, —O—(C₁₋₆)alkyl, COOH, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
      wherein each of the aryl and Het is optionally substituted with 1 to 3 substituents each independently selected from:
      i) halo, cyano, oxo, —OH, —O—(C₁₋₆)alkyl, (C₁₋₆)haloalkyl, —NH₂, —N((C₁₋₄)alkyl)₂ or —NH—C(═O)(C₁₋₄)alkyl;
      ii) (C₁₋₆)alkyl optionally substituted with —O—(C₁₋₆)alkyl; and
      iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
   c) —N(R⁸)R⁹, —C(═O)—N(R⁸)R⁹, —SO₂—N(R⁸)R⁹ or —(C₁₋₆)alkylene-N(R⁸)R⁹;
      R⁸ is H; and
      R⁹ is in each instance independently selected from R⁷,
      —(C₁₋₆)alkylene-R⁷ or —C(═O)—R⁷, wherein R⁷ is as defined above;
   wherein Het is defined as:

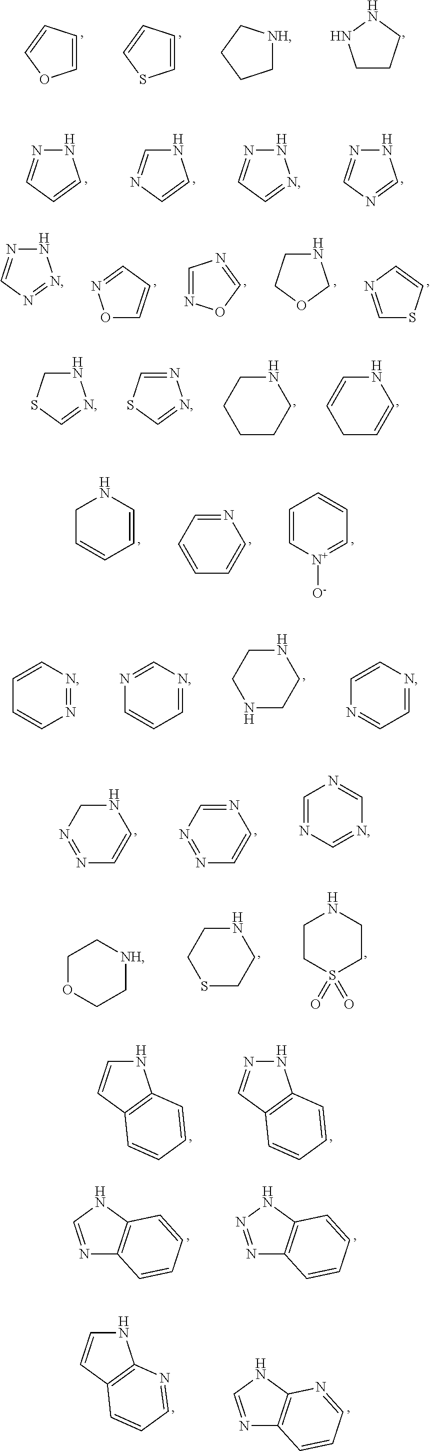

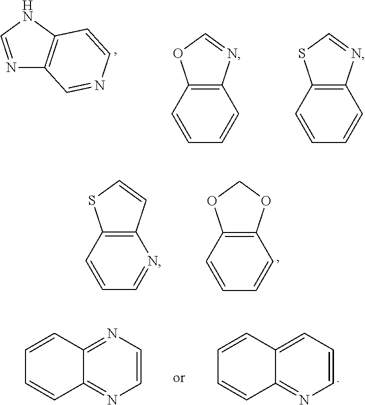

R²-I: In another embodiment, R² is:
   H, F, SO₃H, NO₂, C(═O)OH, C(═O)CH₃, NH₂, CH₃, CF₃, OH, —OCH₃, —CH₂OCH₃, —CH₂OCH₂CH₃, —CH₂OCH(CH₃)₂, —CH₂OCH₂CH(CH₃)₂, —CH₂OH,

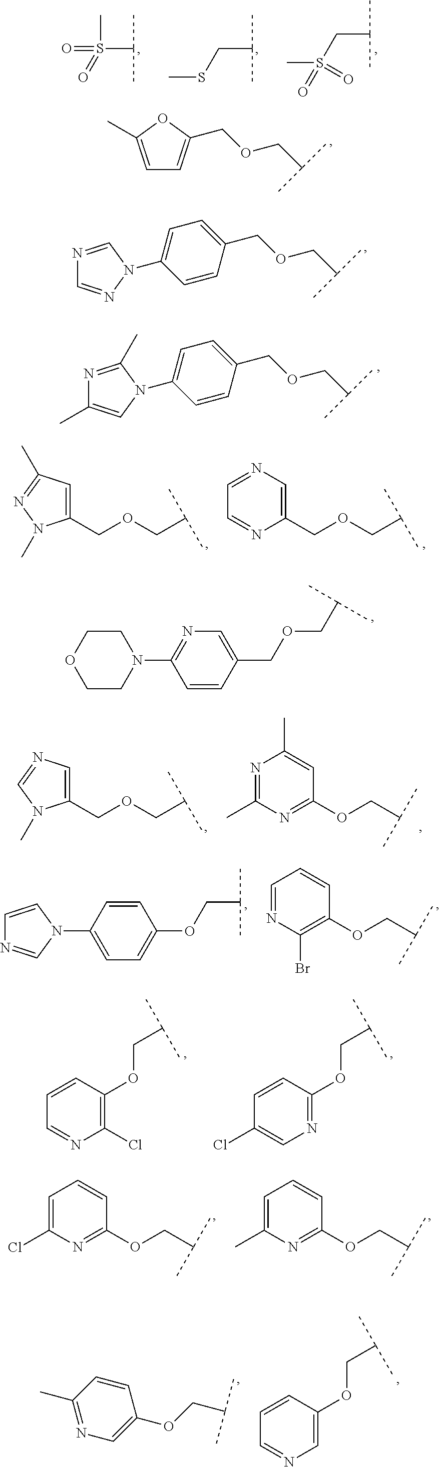

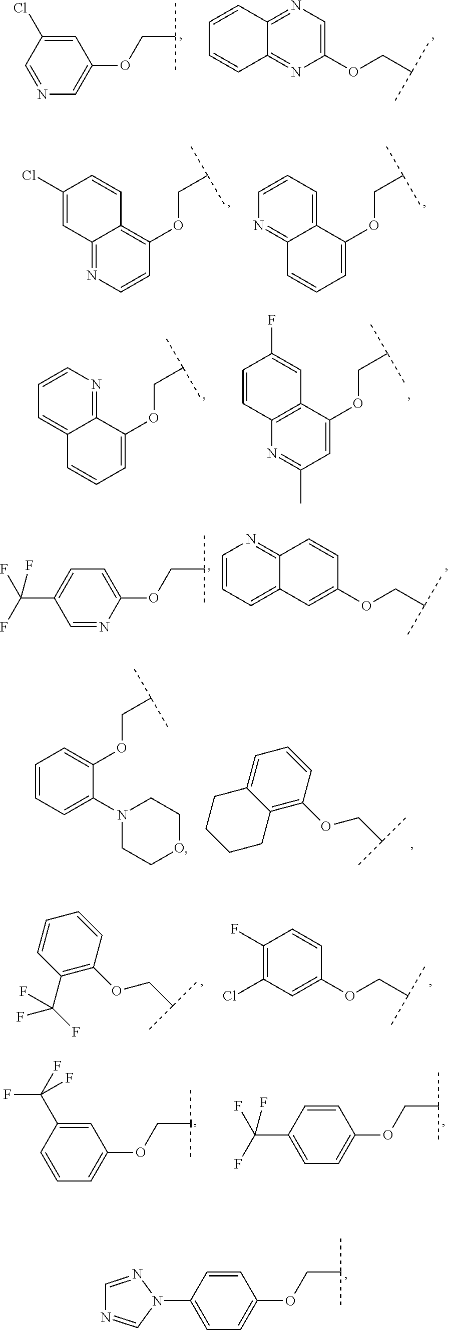

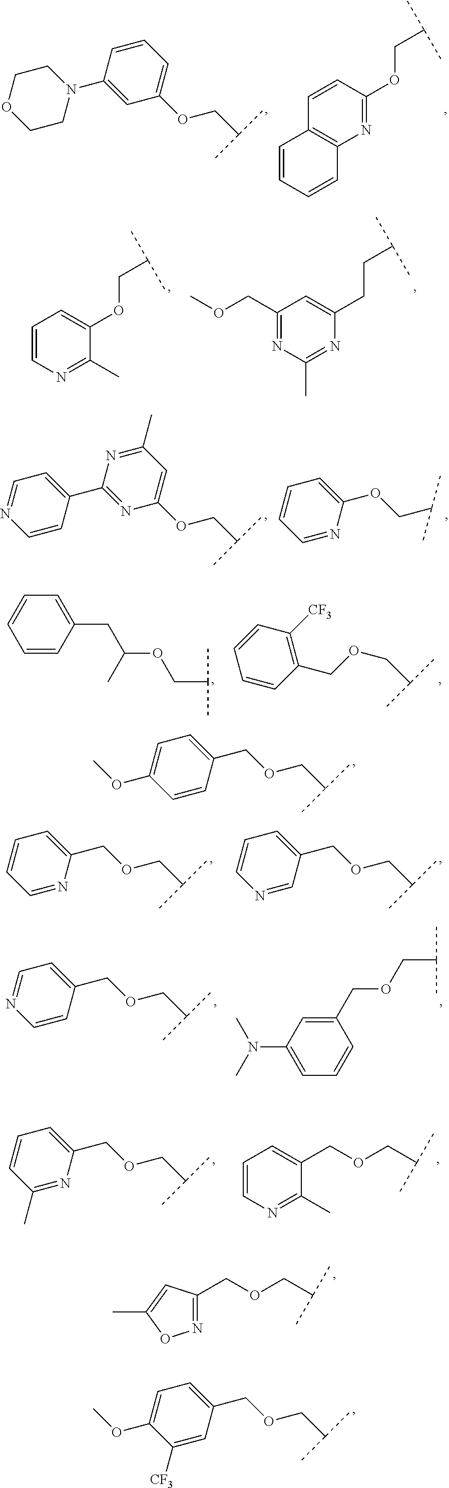

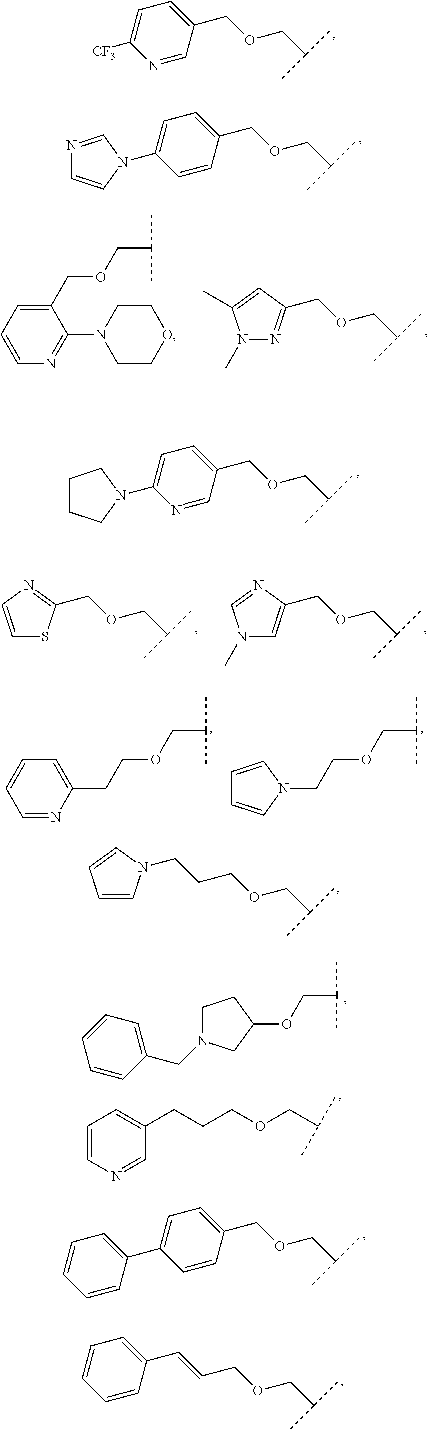

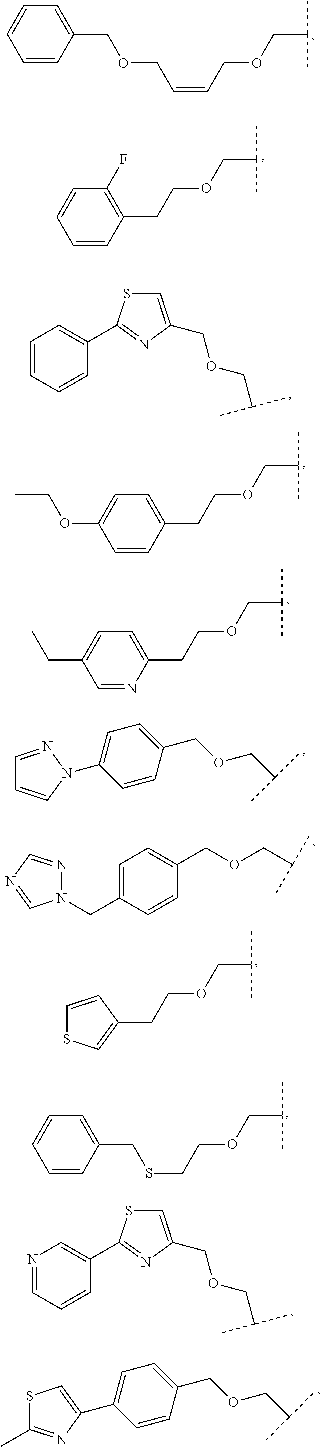

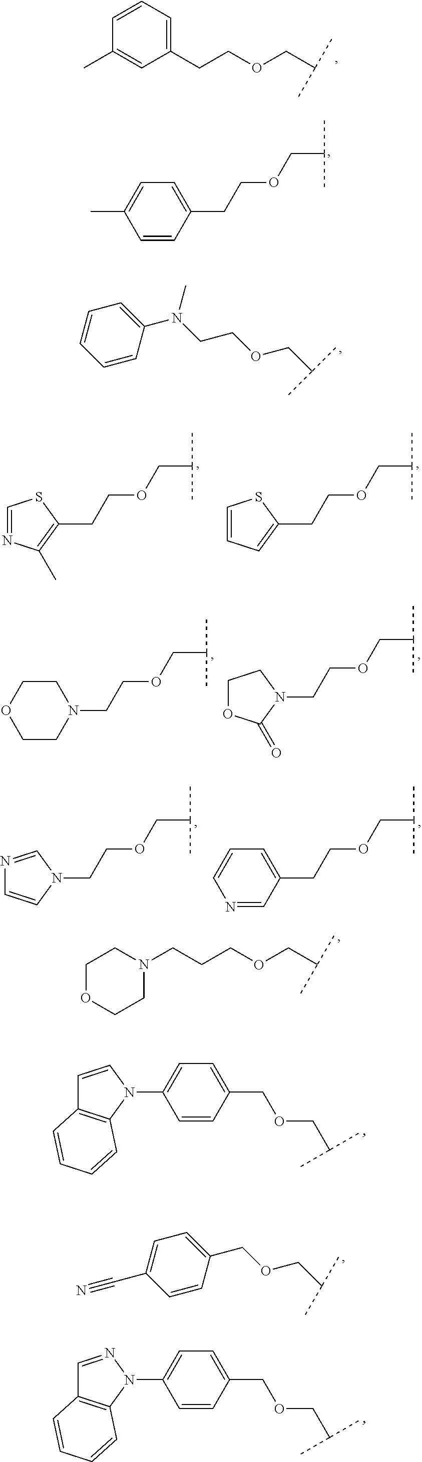

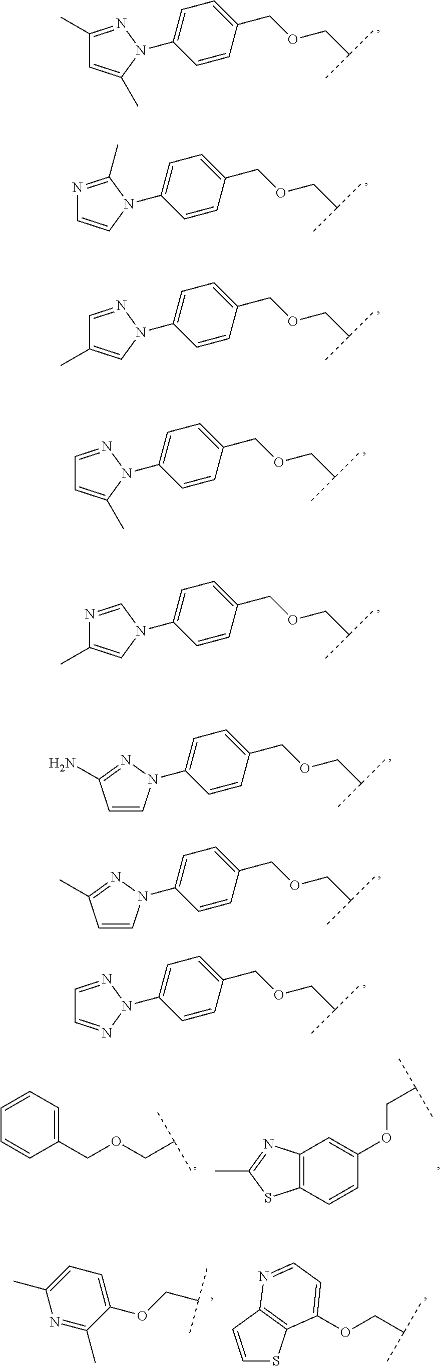

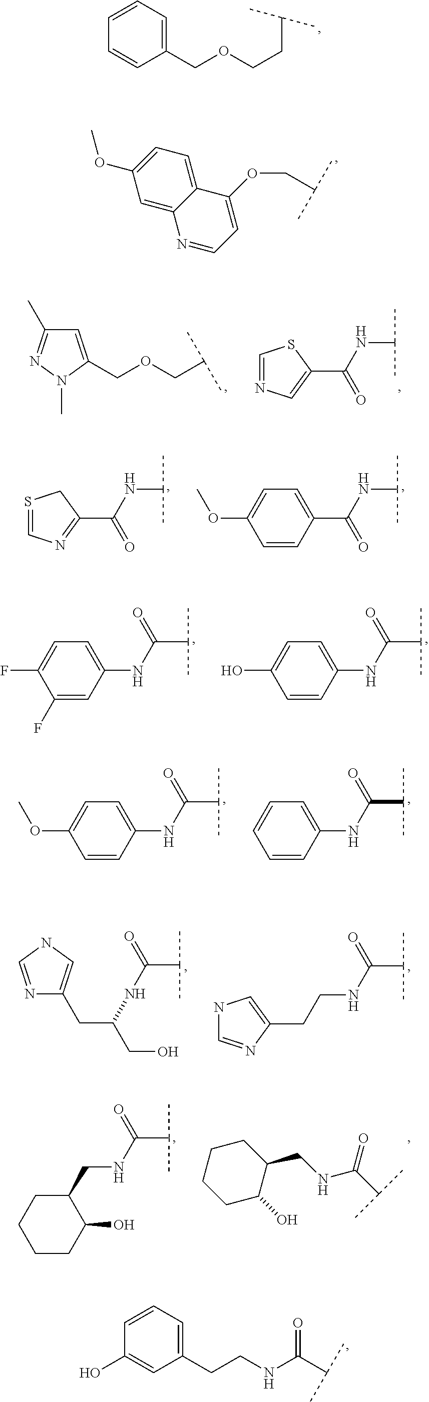

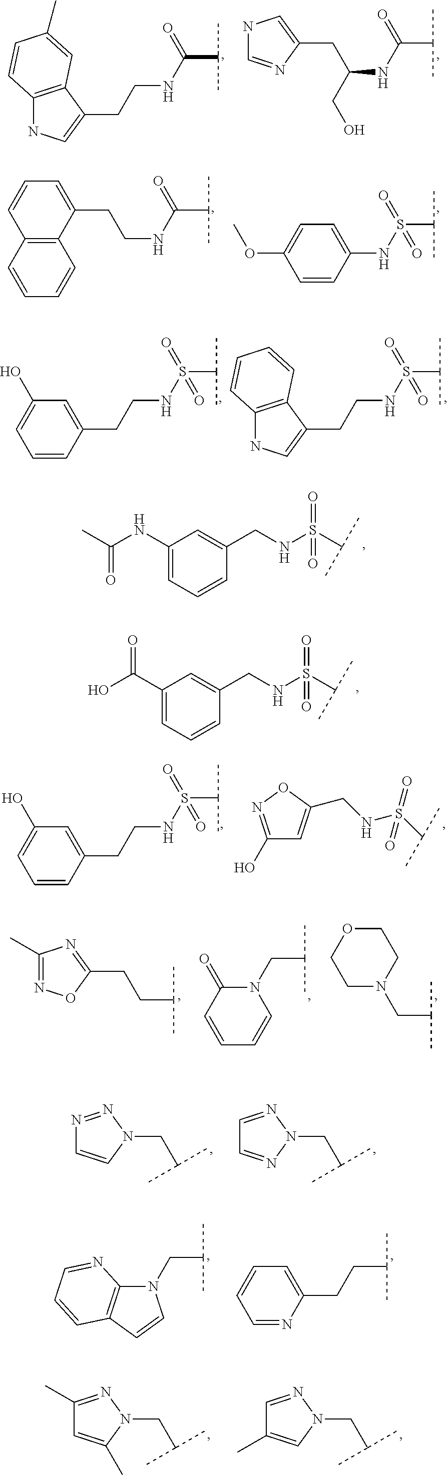

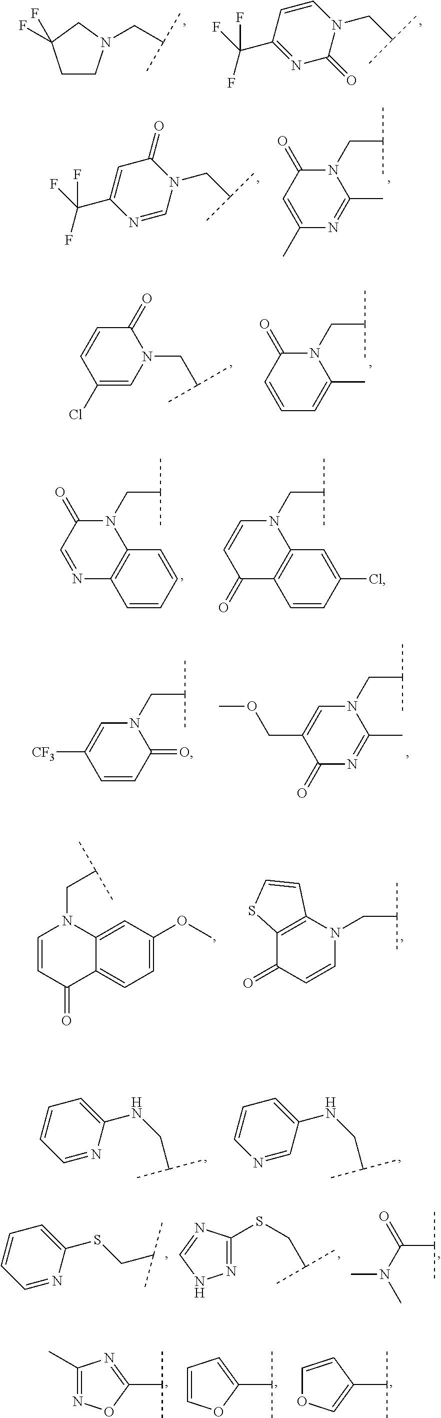

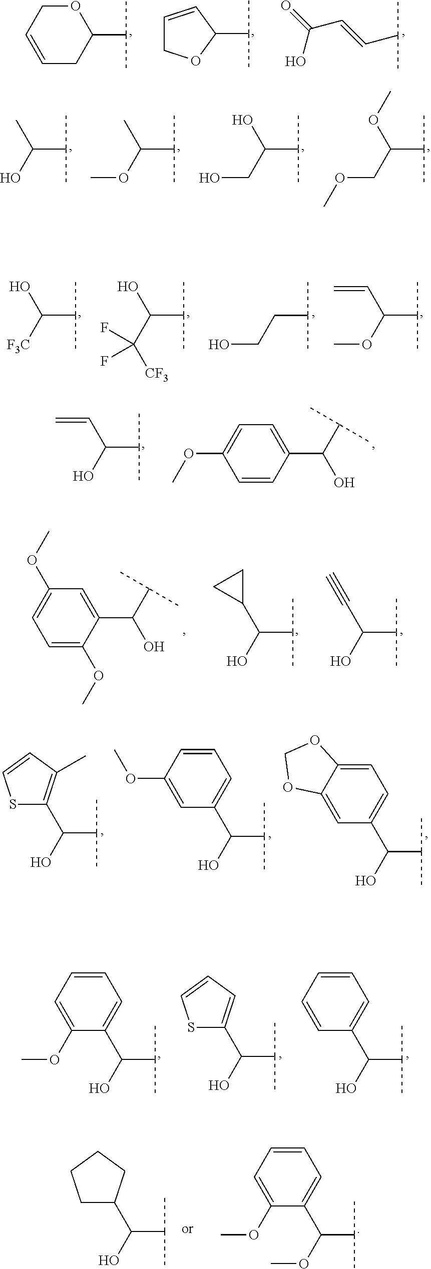

Any and each individual definition of R² as set out herein may be combined with any and each individual definition the Core, n, R⁵ and R⁶ as set out herein,

n:
n-A: In one embodiment; n is 0, 1, 2, 3 or 4,
n-B: In another embodiment, n is 0, 1, 2 or 3,
n-C: In another embodiment, n is 0.1 or 2.
n-D: In another embodiment, n is 0 or 1.

Any and each individual definition of n as set out herein may be combined with any and each individual definition of the Core, R², R⁵ and R⁶ as set out herein.

### R⁵:

R⁵-A: In one embodiment, R⁵ is H, (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het; each being optionally substituted with 1 to 4 substituents each independently selected from (C₁₋₆)alkyl, (C₁₋₆)haloalkyl, (C₃₋₇)cycloalkyl, Het, —OH, —COOH, —C(═O)—(C₁₋₆)alkyl, —C(═O)—O—(C₁₋₆)alkyl, —SO₂(C₁₋₆)alkyl, —C(═O)—N(R⁵¹)R⁵² and —O—R⁵³; wherein R⁵³ is (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het, said aryl and Het being optionally substituted with (C₁₋₆)alkyl or —O—(C₁₋₆)alkyl;
   wherein R⁵¹ is H, (C₁₋₆)alkyl or (C₃₋₇)cycloalkyl; and
   R⁵² is H, (C₁₋₆)alkyl, (C₃₋₇)cycloalkyl, aryl, Het, —(C₁₋₃)alkyl-aryl or —(C₁₋₃)alkyl-Het;
      wherein each of the (C₁₋₆)alkyl, (C₃₋₇)cycloalkyl, aryl, Het, —(C₁₋₃)alkyl-aryl and —(C₁₋₃)alkyl-Het are optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
         wherein the (C₁₋₆)alkyl is optionally substituted with OH; or R⁵¹ and R⁵², together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
      wherein the heterocycle is optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl.
R⁵-B: In another embodiment, R⁵ is H, (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het; each being optionally substituted with 1 to 4 substituents each independently selected from (C₁₋₆)alkyl, (C₁₋₆)haloalkyl, (C₃₋₇)cycloalkyl, Het, —OH, —COOH, —C(═O)—(C₁₋₆)alkyl, —C(═O)—O—(C₁₋₆)alkyl, —SO₂(C₁₋₆)alkyl, —C(═O)—N(R⁵¹)R⁵² and —O—R⁵³; wherein R⁵³ is (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het, said aryl and Het being optionally substituted with (C₁₋₆)alkyl or —O—(C₁₋₆)alkyl; wherein R⁵¹ is H, (C₁₋₆)alkyl or (C₃₋₇)cycloalkyl; and
   R⁵² is H, (C₁₋₆)alkyl, (C₃₋₇)cycloalkyl, aryl, Het, —(C₁₋₃)alkyl-aryl or —(C₁₋₃)alkyl-Het;
      wherein each of the (C₁₋₆)alkyl, (C₃₋₇)cycloalkyl, aryl, Het, —(C₁₋₃)alkyl-aryl and —(C₁₋₃)alkyl-Het are optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
         wherein the (C₁₋₆)alkyl is optionally substituted with OH.
R⁵-C: In another embodiment, R⁵ is (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het; each being optionally substituted with 1 to 4 substituents each independently selected from (C₁₋₆)alkyl, (C₁₋₆)haloalkyl, (C₃₋₇)cycloalkyl, Het, —OH, —COOH, —C(═O)—(C₁₋₆)alkyl, —C(═O)—O—(C₁₋₆)alkyl, —SO₂(C₁₋₆)alkyl, —C(═O)—N(R⁵¹)R⁵² and —O—R⁵³; wherein R⁵³ is (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het, said aryl and Het being optionally substituted with (C₁₋₆)alkyl or —O—(C₁₋₆)alkyl;
   wherein R⁵¹ is H, (C₁₋₆)alkyl or (C₃₋₇)cycloalkyl; and
   R⁵² is H, (C₁₋₆)alkyl, (C₃₋₇)cycloalkyl, aryl, Het, alkyl-aryl or —(C₁₋₃)alkyl-Het;
      wherein each of the (C₁₋₆)alkyl, (C₃₋₇)cycloalkyl, aryl, Het, —(C₁₋₃)alkyl-aryl and —(C₁₋₃)alkyl-Het are optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
         wherein the (C₁₋₆)alkyl is optionally substituted with OH.
R⁵-D: In another embodiment, R⁵ is (C₁₋₆)alkyl, (C₃₋₇)cycloalkyl, —(C₁₋₆)alkyl-(C₃₋₇)cycloalkyl; each being optionally substituted with 1 to 4 substituents each independently selected from (C₁₋₆)alkyl, (C₁₋₆)haloalkyl, (C₃₋₇)cycloalkyl, Het, —OH, —COOH, —C(═O)—(C₁₋₆)alkyl, —C(═O)—O—(C₁₋₆)alkyl, —SO₂(C₁₋₆)alkyl, —C(═O)—N(R⁵¹)R⁵² and —O—R⁵³; wherein R⁵³ is (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het, said aryl and Het being optionally substituted with (C₁₋₆)alkyl or —O—(C₁₋₆)alkyl; wherein R⁵¹ is H, (C₁₋₆)alkyl or (C₃₋₇)cycloalkyl; and
   R⁵² is H, (C₁₋₆)alkyl, (C₃₋₇)cycloalkyl, aryl, Het, —(C₁₋₃)alkyl-ary or —(C₁₋₃)alkyl-Het;
      wherein each of the (C₁₋₆)alkyl, (C₃₋₇)cycloalkyl, aryl, Het, —(C₁₋₃)alkyl-aryl and (C₁₋₃)alkyl-Het are optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
         wherein the (C₁₋₆)alkyl is optionally substituted with OH.
R⁵-E: In one embodiment, R⁵ is (C₁₋₆)alkyl, (C₃₋₇)cycloalkyl, —(C₁₋₆)alkyl-C₃₋₇)cycloalkyl; each being optionally substituted with 1 to 2 substituents each independently selected from (C₁₋₆)alkyl, —OH, —C(═O)—(C₁₋₆)alkyl, —C(═O)—O—(C₁₋₆)alkyl, —C(═O)—N(R⁵¹)R⁵² and —O—R⁵³; wherein R⁵³ is (C₁₋₆)alkyl, (C₃₋₇)cycloalkyl or —(C₁₋₆)alkyl-(C₃₋₇)cycloalkyl; R⁵¹ is H, (C₁₋₆)alkyl or (C₃₋₇)cycloalkyl; and R⁵² is H, (C₁₋₆)alkyl or (C₃₋₇)cycloalkyl.
R⁵-F: In one embodiment, R⁵ is (C₁₋₆)alkyl or (C₃₋₇)cycloalkyl; each being optionally substituted with 1 to 2 substituents each independently selected from (C₁₋₄)alkyl, —OH, —C(═O)—(C₁₋₄)alkyl, —C(═O)—O—(C₁₋₄)alkyl, —C(═O)—N(R⁵¹)R⁵² and —O—(C₁₋₆)alkyl; R⁵¹ is H or (C₁₋₄)alkyl; and R⁵² is H or (C₁₋₄)alkyl.
R⁵-G: In another embodiment, R⁵ is (C₁₋₄)alkyl or (C₃₋₇)cycloalkyl; each being optionally substituted with 1 to 2 substituents each independently selected from (C₁₋₄)alkyl, —C(═O)—N(R⁵¹)R⁵² and —O—(C₁₋₄)alkyl; R⁵¹ is (C₁₋₄)alkyl; and
   R⁵² is (C₁₋₄)alkyl.
R⁵-H: In another embodiment, R⁵ is:

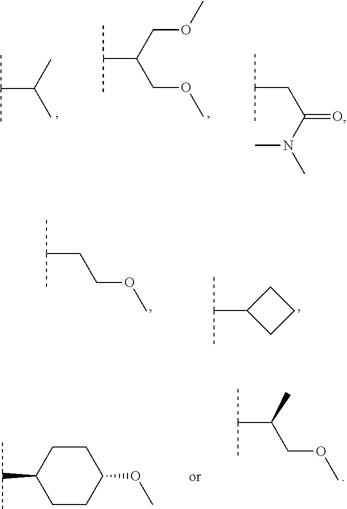

Any and each individual definition of R⁵ as set out herein may be combined with any and each individual definition of the Core, n, R² and R⁶ as set out herein.

### R⁶:

R⁶-A: In one embodiment, R⁶ is (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het; being optionally substituted with 1 to 5 substituents each independently selected from halo, (C₁₋₆)alkyl, (C₁₋₆)haloalkyl, (C₃₋₇)cycloalkyl, —OH, —SH, —O—(C₁₋₄)alkyl, —S—(C₁₋₄)alkyl and —N(R⁸)R⁹; wherein R⁸ is in each instance independently selected from H, (C₁₋₆)alkyl and (C₃₋₇)cycloalkyl; and
   R⁹ is in each instance independently selected from R⁷, —O—(C₁₋₆)alkyl, —(C₁₋₆)alkylene-R⁷, —SO₂—R⁷, —C(═O)—R⁷, —C(═O)OR⁷ and —C(═O)N(R⁸)R⁷;
   wherein R⁷ and R⁸ are as defined above;
   or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
   wherein the heterocycle is optionally substituted with 1 to 3 substituents each independently selected from (C₁₋₆)alkyl, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl.
R⁶-B: In yet another alternative embodiment, R⁶ is (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het, being optionally substituted with 1 to 3 substituents each independently selected from halo, (C₁₋₆)alkyl and (C₁₋₆)haloalkyl.
R⁶-C: In still another embodiment, R⁶ is (C₃₋₇)cycloalkyl, —(C₁₋₆)alkyl-(C₃₋₇)cycloalkyl, phenyl or Het, optionally substituted with 1 to 3 substituents each independently selected from halo, (C₁₋₄)alkyl and (C₁₋₄)haloalkyl;
   wherein the Het is selected from:

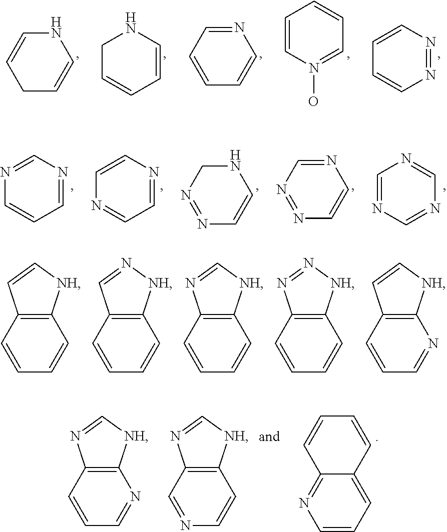

R⁶-D: In another alternative embodiment. R⁶ is (C₅₋₆)cycloalkyl, —(C₁₋₃)alkyl-(C₅₋₆)cycloalkyl, phenyl or Het optionally substituted with 1 to 3 substituents each independently selected from halo, (C₁₋₄)alkyl and (C₁₋₄)haloalkyl; wherein Het is a 4- to 7-membered saturated, unsaturated or aromatic heterocycle having 1 to 3 nitrogen heteroatoms.
R⁶-E: In still another embodiment, R⁶ is phenyl, cyclohexyl, —CH₂-cyclopentyl or pyridine optionally substituted with 1 to 3 substituents each independently selected from halo, (C₁₋₄)alkyl and (C₁₋₄)haloalkyl.
R⁶-F: In still another embodiment, R⁶ is phenyl, optionally substituted with 1 to 3 substituents each independently selected from halo and (C₁₋₄)alkyl.
R⁶-G: In still another embodiment, R⁶ is pyridine, optionally substituted with 1 to 3 substituents each independently selected from halo and (C₁₋₄)alkyl.
R⁶-H: In still another embodiment, R⁶ is cyclohexyl or —CH₂-cyclopentyl, optionally substituted with 1 to 3 substituents each independently selected from halo, (C₁₋₄)alkyl and (C₁₋₄)haloalkyl.
R⁶-I: In still another embodiment, R⁶ is:

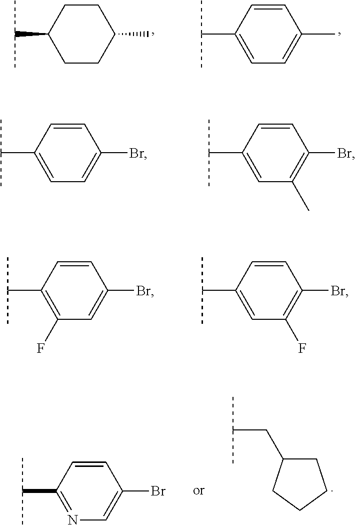

Any and each individual definition of R⁶ as set out herein may be combined with any and each individual definition of the Core, n, R², and R⁵ as set out herein.

Examples of preferred subgeneric embodiments of the present invention are et forth in the following table, wherein each substituent group of each embodiment is defined according to the definitions set forth above:

| Embodiment | Core | n | R² | R⁵ | R⁶ |
|---|---|---|---|---|---|
| E-1 | Core-A | n-C | R²-G | R⁵-E | R⁶-H |
| E-2 | Core-A | n-C | R²-D | R⁵-E | R⁶-H |
| E-3 | Core-A | n-B | R²-E | R⁵-E | R⁶-H |
| E-4 | Core-A | n-B | R²-G | R⁵-G | R⁶-D |
| E-5 | Core-A | n-C | R²-D | R⁵-G | R⁶-D |
| E-6 | Core-A | n-C | R²-E | R⁵-G | R⁶-D |
| E-7 | Core-A | n-A | R²-G | R⁵-E | R⁶-H |
| E-8 | Core-B | n-C | R²-G | R⁵-E | R⁶-D |
| E-9 | Core-B | n-C | R²-D | R⁵-E | R⁶-D |
| E-10 | Core-B | n-C | R²-E | R⁵-E | R⁶-D |
| E-11 | Core-B | n-C | R²-G | R⁵-E | R⁶-E |
| E-12 | Core-B | n-C | R²-D | R⁵-E | R⁶-E |
| E-13 | Core-B | n-C | R²-E | R⁵-E | R⁶-E |
| E-14 | Core-B | n-C | R²-G | R⁵-E | R⁶-H |
| E-15 | Core-B | n-B | R²-D | R⁵-E | R⁶-H |
| E-16 | Core-B | n-C | R²-E | R⁵-E | R⁶-H |
| E-17 | Core-B | n-C | R²-G | R⁵-G | R⁶-D |
| E-18 | Core-B | n-B | R²-D | R⁵-G | R⁶-D |
| E-19 | Core-B | n-C | R²-E | R⁵-G | R⁶-D |
| E-20 | Core-B | n-C | R²-G | R⁵-G | R⁶-E |
| E-21 | Core-B | n-C | R²-D | R⁵-G | R⁶-E |
| E-22 | Core-B | n-C | R²-E | R⁵-G | R⁶-E |
| E-23 | Core-B | n-C | R²-G | R⁵-G | R⁶-H |
| E-24 | Core-B | n-C | R²-D | R⁵-G | R⁶-H |
| E-25 | Core-B | n-C | R²-E | R⁵-G | R⁶-H |
| E-26 | Core-B | n-D | R²-A | R⁵-C | R⁶-B |
| E-27 | Core-B | n-D | R²-A | R⁵-H | R⁶-C |
| E-28 | Core-B | n-C | R²-C | R⁵-E | R⁶-D |
| E-29 | Core-B | n-B | R²-D | R⁵-D | R⁶-B |
| E-30 | Core-B | n-D | R²-G | R⁵-A | R⁶-A |
| E-31 | Core-B | n-B | R²-H | R⁵-A | R⁶-I |
| E-32 | Core-B | n-B | R²-D | R⁵-B | R⁶-C |
| E-33 | Core-B | n-B | R²-E | R⁵-C | R⁶-C |
| E-34 | Core-C | — | R²-G | R⁵-E | R⁶-D |
| E-35 | Core-C | — | R²-D | R⁵-E | R⁶-D |
| E-36 | Core-C | — | R²-E | R⁵-E | R⁶-D |
| E-37 | Core-C | — | R²-G | R⁵-E | R⁶-E |
| E-38 | Core-C | — | R²-D | R⁵-E | R⁶-E |
| E-39 | Core-C | — | R²-E | R⁵-E | R⁶-E |
| E-40 | Core-C | — | R²-G | R⁵-E | R⁶-H |
| E-41 | Core-C | — | R²-D | R⁵-E | R⁶-H |
| E-42 | Core-C | — | R²-E | R⁵-E | R⁶-H |
| E-43 | Core-C | — | R²-G | R⁵-G | R⁶-D |
| E-44 | Core-C | — | R²-D | R⁵-G | R⁶-D |
| E-45 | Core-C | — | R²-E | R⁵-G | R⁶-D |
| E-46 | Core-C | — | R²-G | R⁵-G | R⁶-E |
| E-47 | Core-C | — | R²-D | R⁵-G | R⁶-E |
| E-48 | Core-C | — | R²-E | R⁵-G | R⁶-E |
| E-49 | Core-C | — | R²-G | R⁵-G | R⁶-H |
| E-50 | Core-C | — | R²-D | R⁵-G | R⁶-H |
| E-51 | Core-C | — | R²-E | R⁵-G | R⁶-H |
| E-52 | Core-C | — | R²-E | R⁵-B | R⁶-I |
| E-53 | Core-C | — | R²-D | R⁵-D | R⁶-E |
| E-54 | Core-C | — | R²-I | R⁵-E | R⁶-F |
| E-55 | Core-C | — | R²-A | R⁵-G | R⁶-A |
| E-56 | Core-C | — | R²-B | R⁵-H | R⁶-B |
| E-57 | Core-C | — | R²-F | R⁵-C | R⁶-B |
| E-58 | Core-F | n-C | R²-G | R⁵-G | R⁶-E |
| E-59 | Core-F | n-C | R²-G | R⁵-G | R⁶-H |
| E-60 | Core-F | n-C | R²-E | R⁵-E | R⁶-D |

Examples of most preferred compounds according to this invention are each single compound listed in the following Tables 1 to 3.

In general, all tautomeric and isomeric forms and mixtures thereof, for example, individual geometric isomers, stereoisomers, atropisomers, enantiomers, diastereomers, racemates, racemic or non-racemic mixtures of stereoisomers, mixtures of diastereomers, or mixtures of any of the foregoing forms of a chemical structure or compound is intended, unless the specific stereochemistry or isomeric form is specifically indicated in the compound name or structure. Compounds of the invention that contain asymmetrically substituted carbon atoms can be isolated in optically active or racemic forms.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the present invention from this disclosure and the knowledge in the art.

Preparation of pure stereoisomers, e.g. enantiomers and diastereomers, or mixtures of desired enantiomeric excess (ee) or enantiomeric purity, are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Abuja, Chiral Separations by Chromatography, Am. Chem. Soc., 2000, herein incorporated by reference. Furthermore, there are equally well-known methods for the quantitation of enantiomeric excess or purity, for example, GC, HPLC, CE, or NMR, and assignment of absolute configuration and conformation, for example, CD, ORD, X-ray crystallography, or NMR.

The compounds according to the present invention are inhibitors of the hepatitis C virus NS5B RNA-dependent RNA polymerase and thus may be used to inhibit replication of hepatitis C viral RNA.

A compound according to the present invention may also be used as a laboratory reagent or a research reagent. For example, a compound of the present invention may be used as positive control to validate assays, including but not limited to surrogate cell-based assays and in vitro or in vivo viral replication assays.

Compounds according to the present invention may also be used as probes to study the hepatitis C virus NS5B polymerase, including but not limited to the mechanism of action of the polymerase, conformational changes undergone by the polymerase under various conditions and interactions with entities which bind to or otherwise interact with the polymerase.

Compounds of the invention used as probes may be labelled with a label which allows recognition either directly or indirectly of the compound such that it can be detected, measured and quantified. Labels contemplated for use with the compounds of the invention include, but are not limited to, fluorescent labels, chemiluminescent labels, calorimetric labels, enzymatic markers, radioactive isotopes, affinity tags and photoreactive groups.

Compounds of the invention used as probes may also be labelled with an affinity tag whose strong affinity for a receptor can be used to extract from a solution the entity to which the ligand is attached. Affinity tags include but are not limited to biotin or a derivative thereof, a histidine polypeptide, a polyarginine, an amylose sugar moiety or a defined epitope recognizable by a specific antibody.

Furthermore, compounds of the invention used as probes may be labelled with a photoreactive group which is transformed, upon activation by light, from an inert group to a reactive species, such as a free radical. Photoreactive groups include but are not limited to photoaffinity labels such as benzophenone and azide groups.

Furthermore, a compound according to the present invention may be used to treat or prevent viral contamination of materials and therefore reduce the risk of viral infection of laboratory or medical personnel or patients who come in contact with such materials (e.g. blood, tissue, surgical instruments and garments, laboratory instruments and garments, and blood collection apparatuses and materials).

### Pharmaceutical Composition

Compounds of the present invention may be administered to a mammal in need of treatment for hepatitis C viral infection as a pharmaceutical composition comprising a therapeutically effective amount of a compound according to the invention or a pharmaceutically acceptable salt or ester thereof; and one or more conventional non-toxic pharmaceutically-acceptable carriers, adjuvants or vehicles. The specific formulation of the composition is determined by the solubility and chemical nature of the compound, the chosen route of administration and standard pharmaceutical practice. The pharmaceutical composition according to the present invention may be administered orally or systemically.

For oral administration, the compound, or a pharmaceutically acceptable salt or ester thereof, can be formulated in any orally acceptable dosage form including but not limited to aqueous suspensions and solutions, capsules, powders, syrups, elixirs or tablets. For systemic administration, including but not limited to administration by subcutaneous, intracutaneous, intravenous, intramuscular, intra-articular, intrasynovial, intrasternal, intrathecal, and intralesional injection or infusion techniques, it is preferred to use a solution of the compound, or a pharmaceutically acceptable salt or ester thereof, in a pharmaceutically acceptable sterile aqueous vehicle.

Pharmaceutically acceptable carriers, adjuvants, vehicles, excipients and additives as well as methods of formulating pharmaceutical compositions for various modes of administration are well-known to those of skill in the art and are described in pharmaceutical texts such as Remington: The Science and Practice of Pharmacy, 21st Edition, Lippincott Williams & Wilkins, 2005; and L. V. Allen, N. G. Popovish and H. C. Ansel, Pharmaceutical Dosage Forms and Drug Delivery Systems, 8th ed., Lippincott Williams & Wilkins, 2004, herein incorporated by reference.

The dosage administered will vary depending upon known factors, including but not limited to the activity and pharmacodynamic characteristics of the specific compound employed and its mode, time and route of administration; the age, diet, gender, body weight and general health status of the recipient; the nature and extent of the symptoms; the severity and course of the infection; the kind of concurrent treatment; the frequency of treatment; the effect desired; and the judgment of the treating physician; in general, the compound is most desirably administered at a dosage level that will generally afford antivirally effective results without causing any harmful or deleterious side effects.

A daily dosage of active ingredient can be expected to be about 0.01 to about 200 milligrams per kilogram of body weight, with the preferred dose being about 0.1 to about 50 mg/kg. Typically, the pharmaceutical composition of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

### Combination Therapy

Combination therapy is contemplated wherein a compound according to the invention, or a pharmaceutically acceptable salt or ester thereof, is co-administered with at least one additional antiviral agent. The additional agents may be combined with compounds of this invention to create a single dosage form. Alternatively these additional agents may be separately administered, concurrently or sequentially, as part of a multiple dosage form.

When the pharmaceutical composition of this invention comprises a combination of a compound according to the invention, or a pharmaceutically acceptable salt or ester thereof, and one or more additional antiviral agent, both the compound and the additional agent should be present at dosage levels of between about 10 to 100%, and more preferably between about 10 and 80% of the dosage normally administered in a monotherapy regimen. In the case of a synergistic interaction between the compound of the invention and the additional antiviral agent or agents, the dosage of any or all of the active agents in the combination may be reduced compared to the dosage normally administered in a monotherapy regimen.

Antiviral agents contemplated for use in such combination therapy include agents (compounds or biologicals) that are effective to inhibit the formation and/or replication of a virus in a mammal, including but not limited to agents that interfere with either host or viral mechanisms necessary for the formation and/or replication of a virus in a mammal. Such agents can be selected from another anti-HCV agent; an HIV inhibitor, an HAV inhibitor, and an HBV inhibitor.

Other anti-HCV agents include those agents that are effective for diminishing or preventing the progression of hepatitis C related symptoms or disease. Such agents include but are not limited to immunomodulatory agents, inhibitors of HCV NS3 protease, other inhibitors of HCV polymerase, inhibitors of another target in the HCV life cycle and other anti-HCV agents, including but not limited to ribavirin, amantadine, levovirin and viramidine.

Immunomodulatory agents include those agents (compounds or biologicals) that are effective to enhance or potentiate the immune system response in a mammal. Immunomodulatory agents include, but are not limited to, inosine monophosphate dehydrogenase inhibitors such as VX-497 (merimepodtb, Vertex Pharmaceuticals), class I interferons, class II interferons, consensus interferons, asialo-interferons pegylated interferons and conjugated interferons, including but not limited to interferons conjugated with other proteins including but not limited to human albumin. Class I interferons are a group of interferons that all bind to receptor type I, including both naturally and synthetically produced class I interferons, while class II interferons all bind to receptor type II. Examples of class I interferons include, but are not limited to, α-, β-, δ-, ω-, and τ-interferons, while examples of class II interferons include, but are not limited to, γ-interferons. In one preferred aspect, the other anti-HCV agent is an interferon. Preferably, the interferon is selected from the group consisting of interferon alpha 2B, pegylated interferon alpha, consensus interferon, interferon alpha 2A and lymphoblastoid interferon, in one preferred aspect, the composition comprises a compound of the invention, an interferon and ribavirin.

Inhibitors of HCV NS3 protease include agents (compounds or biologicals) that are effective to inhibit the function of HCV NS3 protease in a mammal. Inhibitors of HCV NS3 protease include, for example, those compounds described in WO 99/07733, WO 99/07734, WO 00/09558, WO 00/09543, WO 00/59929, WO 03/064416, WO 03/064455, WO 03/064456, WO.2004/039970, WO 2004/037855, WO 2004/039833, WO 2004/101602, WO 2004/101605, WO 2004/103996, WO 2005/028501, WO 2005/070955, WO 2006/000085, WO 2006/007700, WO 2006/007708, WO 2007/009227, WO 2004/093915, WO 2004/009121 (all by Boehringer Ingelheim), WO 2006/122188, WO 2006/086381, WO 2007/044933, WO 2007/056120, WO 2008/057875, US 2004/077551, WO 2007/008657, WO 2008/064061, WO 2008/064057, WO2008/008776, US 2004/0048802, WO 2008/064066, WO 2008/060927, WO 2008/057871, WO 2008/057873, US 2002/0177725, WO 02/48157, WO 02/48116, WO 2007/001406, WO 2006/101538, WO 02/08251, WO01/02424, WO 01/40262, WO 01/07407, WO 01/64678, WO 02/18369, WO 98/46597, US 2005/0153877, WO 02/060926, WO 03/053349, WO 03/099274, WO 03/099316, WO 2004/032827, WO 2004/043339, WO 2004/094452, WO 2005/046712, WO 2005/051410, WO 2005/054430 (all by BIAS). US 2008/0032936, WO 2008/021960, WO 2008/002924, WO 2007/146695, WO 2007/143694, WO 2006/021733, WO 2008/019289, WO 2008/022006, WO 2008/021956, WO 2008/019266, WO 2008/019,303, WO 2004/072243, WO 2004/093798, WO 2004/113365, WO 2005/010029 (all by Enanta). WO 2005/095403, WO 2008/005511, WO 2007/015824, WO 2007/044893, WO 2006/037214 (intermune). WO 01/58929, U.S. Pat. No. 5,990,276, WO 97/43310, WO 01/77113, WO 2006/130628, US 2003/0216325, US 2005/0176648, US 2005/0209164, WO 01/77113, WO 01/81325, WO 02/08187, WO 02/08198, WO 02/08244, WO 02/08256, WO 02/48172, WO 03/062228, WO 03/062285, WO 2005/021584, WO 2005/030796, WO 2005/058821, WO 2005/051980, WO 2005/085197, WO 2005/085242, WO 2005/085275, WO 2005/087721, WO 2005/087725, WO 2005/087730, WO 2005/087731, WO 2005/107745 and WO 2005/113581 (all by Schering), WO 2008/057209, WO 2008/051475, WO 2006/119061, WO 2007/016441, WO 2007/015855, WO 2007/015787 (all by Merck), WO 2006/043145 (Pfizer), all of which are herein incorporated by reference; and the candidates VX-950, SCH-503034, ITMN-191, TMC 435350, and MK7009.

Inhibitors of HCV polymerase include agents (compounds or biologicals) that are effective to inhibit the function of an HCV polymerase. Such inhibitors include, but are not limited to, non-nucleoside and nucleoside inhibitors of NS4A, NS5A, NS5B polymerase. Examples of inhibitors of HCV polymerase include but are not limited to those compounds described in: WO 03/007945, WO 03/010140, WO 03/010141, U.S. Pat. No. 6,448,281, WO 02/04426, WO 2008/019477, WO 2007/087717, WO 2006/007693, WO 2005/080388, WO 2004/099241, WO 2004/065367, WO 2004/064925 (all by Boehringer Ingelheim), WO 2006/093801, US 2005/0107364, WO 2005/019191, US 2004/0167123, WO 2004/041818, WO 2008/011337 (all by Abbott Laboratories), WO 01/32153 (Biochem Pharma Inc.), WO 01/60315 (Biochem Pharma Inc.), US 2004/0138170, WO 2004/106350, WO 2006/050161, WO 2006/104945, WO 2006/002231, US 2005/080053, US 2004/0242599, US 2004/0229839, WO 03/087298, WO 02/069903 (all by Biocryst Pharmaceuticals, Inc), WO 2006/094347 (Biota, Inc.), WO 2005/021568 (Biota, Inc.), WO 2008/051637, WO 2007/150001, WO 2006/066079 (all by Anadys Pharmaceuticals), WO 2007/033032, WO 2007/033175, WO 03/026587, WO 2007/143521, WO 2007/140109, WO 2007/140200, WO 2007/140254, WO 2007/136982, WO 2007/092000, WO 2007/092888, WO 2006/020082, US 2005/0119318, WO 2005/034850 (all by Bristol-Myers Squibb), WO 2007/034127 (Arrow Therapeutics Limited), WO 2005/063734 (Bayer), WO 03/093290, WO 2005/012288, WO 2008/011521, WO 2008/008907, WO 2008/008912, WO 2007/084157, WO 2007/019397, WO 2006/138744, WO 2006/121468, WO 2006/116557, WO 2006/102594, WO 2006/076529, WO 2006/075993, US 2006/0111311, WO 2005/054268, WO 2005/042556, US 2005/0090463, WO 2004/108687, WO 2004/028481, WO 2006/093986, WO 2006/093987 (all by Genelabs Technologies), WO 2006/117306, WO 2004/046159, WO 2007/113159, WO 2007/093541, WO 2007/068615, WO 2007/065829, WO 2007/020193, WO 2006/021341, WO 2006/021340, WO 02/100415, WO 02/094289, WO 02/18404 (all by F. Hoffmann-La Roche), WO 2007/039142, WO 2007/039145, WO 2007/039144, WO 2006/045613, WO 2006/045615, WO 2005/103045, WO 2005/092863, WO 2005/079799, WO 2004/096774, WO 2004/096210, WO 2004/076415, WO 2004/060889, WO 2004/037818, WO 2004/009543, WO 03/097646, WO 03/037893, WO 03/037894, WO 03/037895, WO 03/000713 (all by Glaxo Group), WO 2007/144686, WO 2006/000922, WO 2004/046331, WO 2004/002422, WO 2004/002999, WO 2004/003000, WO 2005/009418, WO 03/026675, WO 03/026589, WO 2007/025043 (all by Idenix). US03/050320, WO 2007/119889, WO 2006/052013, WO 2005/080399, WO 2005/049622, WO 2005/014543, EP 1 162 196, WO 01/47883 (all by Japan Tobacco), WO 2007/095269, WO 2007/054741, WO 03/062211, WO 00/06529, WO 99/64442, WO 2006/119975, WO 2006/046030, WO 2006/046039, WO 2005/034941, WO 2005/023819, WO 2004/110442, WO 2004/087714, WO 2007/065883, WO 02/06246, WO 2007/129119, WO 2007/029029, WO 2007/028789, WO 2006/029912, WO 2006/027628, WO 2006/008556 (all by Istituto Di Richerche Di Biologia Malecolare P. Angeletti SPA), WO 2008/005542, WO 2006/091905, WO 2005/063751, WO 2004/005286 (all by Gilead Sciences), WO 2008/043704 (Medivir), WO 2005/123087, WO 2007/021610, WO 2006/065335, WO 2006/012078, WO 2004/003138, WO 2004/000858, WO 03/105770, WO 03/020222, WO 2005/084192, WO 2004/009020, WO 2004/007512, WO 02/057425, WO 02/057287, WO 2007/022073, US 2004/0229840 (all by Merck and Co.), WO 2006/018725, WO 2004/073599, WO 2004/074270, WO 03/095441, WO 03/082848 (all by Pfizer), US 2005/00154056, WO 2004/002977. WO 2004/002944, WO 2004/002940 (all by Pharmacia & Upjohn Company), WO 00/04141 (Ribozyme), WO 2006/050035 (Schering), WO 2006/050034 (Schering), US 2003/0203948 (Shionogi), WO 02/20497 (Shionogi), WO 2005/121132 (Shionogi) EP 1321463 (Shire Biochem), WO 02/100851 (Shire Biochem), WO 02/100846 (Shire Biochem), WO 03/061385, WO 03/0162256, WO 03/062255, U.S. Pat. No. 6,906,190, WO 2004/080466 (all by Ribapharm), WO 2007/026024 (Tibotec), WO 2006/065590 (XTL Biopharmaceuticals), WO 2008/051244, WO 2007/092558, WO 2006/034337, WO 03/099275, WO 03/099824 (all by Wyeth), WO 03/059356, WO 01/85172, WO 01/85720, WO 03/037262, WO 2008/059042, WO 2008/043791. WO 2008/017688, WO 2007/147794, WO 2007/088148, WO 2007/071434, WO 2007/039146, WO 2006/100106, WO 2004/058150, WO 2004/052312, WO 2004/052313, WO 03/099801, WO 02/098424 (all by Smithkline Beecham), WO 2007/027248 (Valeant), WO 2008/058393, WO 2006/119646, WO 2004/052879, WO 2004/052885, WO 00/18231, WO 00/13708, WO 00/10573, WO 2004/041201, WO 03/090674 (all by Viropharma), (all of which are herein incorporated by reference) and the candidates HCV 796 (ViroPharma/Wyeth), R-1626, R-1656 and R-7128 (Roche), NM 283 (Idenix/Novartis), VCH-759 and VCH-916 (Virochem), GS9190 (Gilead), GL60667 (Genelabs/Novartis), MK-608 (Merck) and PF868554 (Pfizer).

The term “inhibitor of another target in the HCV life cycle” as used herein means an agent (compound or biological) that is effective to inhibit the formation and/or replication of HCV in a mammal other than by inhibiting the function HCV polymerase. This includes agents that interfere with either host or HCV viral targets necessary for the HCV life cycle or agents which specifically inhibit in HCV cell culture assays through an undefined or incompletely defined mechanism. Inhibitors of another target in the HCV life cycle include, for example, agents that inhibit viral targets such as Core, E1, E2, p7, NS2/3 protease, NS3 helicase, internal ribosome entry site (IRES), HCV entry and HCV assembly or host targets such as cyclophilin B, phosphatidylinositol 4-kinase IIIα, CD81, SR-B1, Claudin 1, VAP-A, VAP-B. Specific examples of inhibitors of another target in the HCV life cycle include ISIS-14803 (ISIS Pharmaceuticals), GS9190 (Gilead), GS9132 (Gilead), A-831 (AstraZeneca), NM-811 (Novartis), and DEBIO-025 (Debio Pharma).

It can occur that a patient may be co-infected with hepatitis C virus and one or more other viruses, including but not limited to human immunodeficiency virus (HIV), hepatitis A virus (HAV) and hepatitis B virus (HBV). Thus also contemplated is combination therapy to treat such co-infections by co-administering a compound according to the present invention with at least one of an HIV inhibitor, an HAV inhibitor and an HBV inhibitor.

HIV inhibitors include agents (compounds or biologicals) that are effective to inhibit the formation and/or replication of HIV. This includes but is not limited to agents that interfere with either host or viral mechanisms necessary for the formation and/or replication of HIV in a mammal HIV inhibitors include, but are not limited to:
NRTIs (nucleoside or nucleotide reverse transcriptase inhibitors) including but not limited to zidovudine (AZT), didanosine (ddI), zalcitabine (ddC), stavudine (d4T), lamivudine (3TC), emtricitabine, abacavir succinate, elvucitabine, adefovir dipivoxil, lobucavir (BMS-180194) lodenosine (FddA) and tenofovir including tenofovir disoproxil and tenofovir disoproxil fumarate salt, COMBIVIR™ (contains 3TC and AZT). TRIZIVIR™ (contains abacavir, 3TC and AZT), TRUVADA™ (contains tenofovir and emtridtabine), EPZICOM™ (contains abacavir and 3TC);
NNRTIs (non-nucleoside reverse transcriptase inhibitors) including but not limited to nevirapine, delaviradine, efavirenz, etravirine and rilpivirine;
protease inhibitors including but not limited to ritonavir, tipranavir, saquinavir, nelfinavir, indinavir, amprenavir, fosamprenavir, atazanavir, lopinavir, darunavir, lasinavir, brecanavir, VX-385 and TMC-114;
entry inhibitors including but not limited to
   CCR5 antagonists (including but not limited to maraviroc, viriviroc, INCB9471 and TAK-652),
   CXCR4 antagonists (including but not limited to AMD-11070),
   fusion inhibitors (including but not limited to enfuvirtide (T-20), TR1-1144 and TR1-999) and
   others (including but not limited to BMS-488043);
integrase inhibitors (including but not limited to raltegravir (MK-0518), BMS 707035 and elvitegravir (GS 9137));
TAT inhibitors;
maturation inhibitors (including but not limited to berivimat (PA-457));
immunomodulating agents (including but not limited to levamisole); and
other antiviral agents including hydroxyurea, ribavirin, IL-2, IL-12 and pensafuside.

HAV inhibitors include agents (compounds or biologicals) that are effective to inhibit the formation and/or replication of HAV. This includes but is not limited to agents that interfere with either host or viral mechanisms necessary for the formation and/or replication of HAV in a mammal. HAV inhibitors include but are not limited to Hepatitis A vaccines.

HBV inhibitors include agents (compounds or biologicals) that are effective to inhibit the formation and/or replication of HBV in a mammal. This includes but is not limited to agents that interfere with either host or viral mechanisms necessary for the formation and/or replication of HBV in a mammal. HBV inhibitors include, but are not limited to agents that inhibit the HBV viral DNA polymerase and HBV vaccines.

Therefore, according to one embodiment, the pharmaceutical composition of this invention additionally comprises a therapeutically effective amount of one or more antiviral agent.

A further embodiment provides the pharmaceutical composition of this invention wherein the one or more antiviral agent comprises at least one other anti-HCV agent.

According to a more specific embodiment of the pharmaceutical composition of this invention, the at least one other anti-HCV agent comprises at least one immunomodulatory agent.

According to another more specific embodiment of the pharmaceutical composition of this invention, the at least one other anti-HCV agent comprises at least one other inhibitor of HCV polymerase.

According to yet another more specific embodiment of the pharmaceutical composition of this invention, the at least one other anti-HCV agent comprises at least one inhibitor of HCV NS3 protease.

According to still another more specific embodiment of the pharmaceutical composition of this invention, the at least one other anti-HCV agent comprises at least one inhibitor of another target in the HCV life cycle.

### EXAMPLES

Other features of the present invention will become apparent from the following non limiting examples which illustrate, by way of example, the principles of the invention. As is well known to a person skilled in the art, reactions are performed in an inert atmosphere (including but not limited to nitrogen or Ar) where necessary to protect reaction components from air or moisture. Preparation of compounds of the invention can involve the protection and deprotection of various chemical groups. The need for protection and deprotection, and the selection of appropriate protecting groups can be readily determined by one skilled in the art. The chemistry of protecting groups can be found, for example, in Greene, “Protective Groups in Organic Chemistry”, John Wiley & Sons, New York (1981), and more recent editions thereof, herein incorporated by reference. Temperatures are given in degrees Celsius (° C.). Solution percentages and ratios express a volume to volume relationship, unless stated otherwise. Flash chromatography is carried out on silica gel (SiO₂) according to the procedure of W. C. Still et al., J. Org. Chem., (1978), 43, 2923. Mass spectral analyses are recorded using electrospray mass spectrometry. Purification on a combiflash is performed using an Isco Combiflash (column cartridge SiO₂). Unless otherwise specified, preparative HPLC is the purification method. Preparative HPLC is carried out under standard conditions using a SunFire™ Prep C18 OBD 5 μM reverse phase column, 19×50 mm and a linear gradient (20 to 98%) employing 0.1% TFA/acetonitrile and 0.1% TFA/water as solvents. Compounds are isolated as TFA salts when applicable. Analytical HPLC is carried out under standard conditions using a Combiscreen™ ODS-AQ C18 reverse phase column, YMC, 50×4.6 mm id., 5 μM, 120 Å at 220 nM, elution with a linear gradient as described in the following table (Solvent A is 0.06% TFA in H₂O solvent B is 0.06% TFA in MeCN):

| Time (min) | Flow (mL/min) | Solvent A (%) | Solvent B (%) |
|---|---|---|---|
| 0 | 3.0 | 95 | 5 |
| 0.5 | 3.0 | 95 | 5 |
| 6.0 | 3.0 | 50 | 50 |
| 10.5 | 3.5 | 0 | 100 |

Abbreviations or symbols used herein include:

Ac: acetyl;
AcOH: acetic acid;
Bn: benzyl (phenylmethyl);
BOC or Boc: tert-butyloxycarbonyl;
Bu: butyl;
DBU: 1,8-diazabicyclo[5.4.0]undec-7-ene;
DCE: dichloroethane;
DCM: dichloromethane;
DIPEA: diisopropylethylamine;
DMA: dimethylacetamide
DMAP: 4-dimethylaminopyridine;

### DMF: N,N-dimethylformamide;

DMSO: dimethylsulfoxide;
EC₅₀: 50% effective concentration;
Et: ethyl;
Et₂O: diethyl ether;
EtOAc: ethyl acetate;
EtOH: ethanol;
Hex: hexane;
HPLC: high performance liquid chromatography;
IC₅₀: 50% inhibitory concentration;
ⁱPr or i-Pr: 1-methylethyl (iso-propyl);
LC-MS: liquid chromatography-mass spectrometry;
Me: methyl;
MeCN: acetonitrile;
MeI: iodomethane;
MeOH: methanol;
MS: mass spectrometry (ES: electrospray);
NaHB(OAc)₃: sodium triacetoxyborohydride;
Ph: phenyl;
Pr: n-propyl;
Psi: pounds per square inch;
Rpm: rotations per minute;
RT: room temperature (approximately 18° C. to 25° C.);
t-BME: tert-butlymethylether
tert-butyl or t-butyl: 1,1-dimethylethyl;
tert-BuOH or t-BuOH: tert-butanol
TBAF: tetrabutylammonium fluoride;
TBTU: 2-(1H-benzotriazole-1-yl)-1,1,3,3-tetramethyl uronium tetrafluoroborate;
TEA: triethylamine;
TFA: trifluoroacetic acid;
THF: tetrahydrofuran;
TLC: thin layer chromatography.

### Example 1A

### Compound 1004

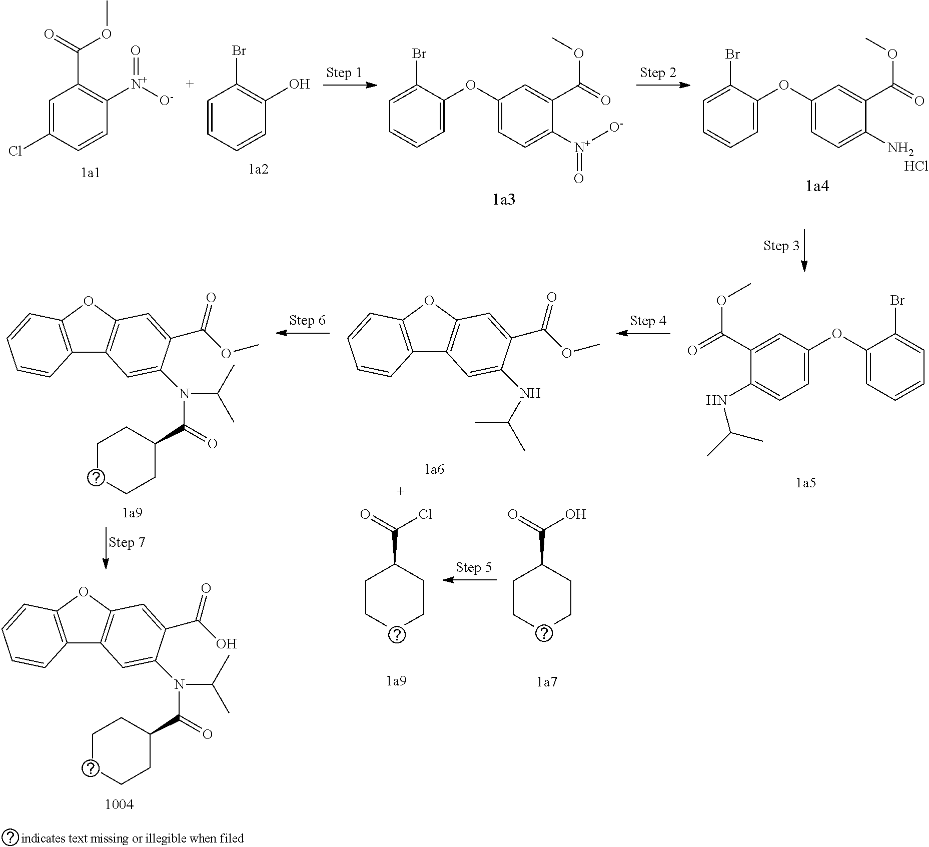

### Step1:

Add anhydrous potassium carbonate (4.83 g, 34.9 mmol) to a mixture of chloro-nitro areae (1a1, 4.98 g, 23.1 mmol) and 2-bromophenol (1a2, 3.4 mL, 29.3 mmol) in DMSO (30 mL) and heat for about 6 h at 80° C. Bring the mixture to RT and leave at RT for about a further 16 h. Dilute the mixture with water (150 mL) and extract with t-BME (3×100 mL). Combine the organic portions, and wash successively with 1 N NaOH (aqueous, 2×50 mL), water (50 mL), and brine (50 mL). Subsequent drying with anhydrous MgSO₄, filtering, and evaporation of the volatiles provides 1a3.

### Step 2:

Dissolve 1a3 (7.00 g, 19.9 mmol) into a mixture consisting of EtOH (80 mL), water (11 mL) and saturated NH₄Cl (aqueous, 11 mL). Add iron powder (3.33 g, 59.6 mmol) in one portion and heat for about 7 h at 80° C. Add another portion of iron powder (2.30 g, 41.2 mmol) to the mixture and continue heating for about a further 9 h. Dilute with EtOAc and filter the solids. Collect the filtrate and evaporate the volatiles. Dissolve the resulting crude material into t-BME and wash successively with saturated NaHCO₃, water, and brine. Dry the organic portion with anhydrous MgSO₄, filter and evaporate the volatiles. Dissolve the resulting material in Et₂O (55 mL) and add 4 N HCl in dioxane (7.5 mL, 30 mmol). Filtering the solids and drying yields 1a4,

### Step 3:

To a suspension of 1a4 (6.35 g, 17.7 mmol) in DCM (50 mL), add 2-methoxypropene (7.00 mL, 73.1 mmol) and NaHB (OAc)₃ (7.97 g, 37.6 mmol). Stir the mixture at RT for about 2.5 h. Dilute with EtOAc and wash the organic portion with water and brine. Dry over MgSO₄, filter and evaporate the volatiles. Purification of the crude material with flash chromatography (EtOAc/Hexanes) provides 1a5.

### Step 4:

Reference: Keith Fagnou et al., J. Org. Chem. 2005, 70, 7578-7584.

Add potassium acetate (680 mg, 5.02 mmol) and Pearlman's catalyst (116 mg, 0.17 mmol) to 1a5 (503 mg, 1.38 mmol) in DMA (10 mL) and heat to 145° C. for about 18 h. Dilute with t-BME containing AcOH (3 mL) and wash with water. Filter the organic portion, dry over Na₂SO₄, filter and evaporate. Dissolve the resulting material in a 0.7 M diazomethane/ether solution and then evaporate the volatiles. Purification of the crude material with flash chromatography (EtOAc/Hexanes) affords 1a6.

### Step

Dissolve trans-4-methylcyclohexane carboxylic acid 1a7 (3.00 g, 21.1 mmol) in DCM (20 mL) and cool to 0° C. Add oxalyl chloride (2.8 mL, 31.6 mmol), then DMF (10 μL). Stir for about 1 h, warm to RT and stir for about a further 3 h. Evaporate the volatiles under reduced pressure and dilute the residue in pentane. The solids are filtered and the filtrate collected. Evaporation of the filtrate to constant mass affords 1a8,

### Step 6:

To a mixture of 1a6 (106 mg, 0.37 mmol) in anhydrous pyridine (3 mL) under an Ar atmosphere, add DMAP (16.8 mg, 0.14 mmol) and 1a8 (250 mg, 1.56 mmol). Heat the mixture to 60° C. for about 17 h. Dilute the mixture with EtOAc and wash successively with 1 N HCl (aqueous), water and brine. Dry the organic phase over MgSO₄, filter and evaporate the volatiles. Purification of the residue with flash chromatography (EtOAc/Hexanes) affords 1a9.

### Step 7;

Add 1 N NaOH (aqueous, 0.4 mL) to a mixture of 1a9 (25 mg, 0.061 mmol) in DMSO (0.4 mL) and heat to 50° C. Subsequently, add MeOH (0.4 mL). After a period of about 5.5 h, add excess TFA until the pH is approximately <2. Purification and lyophilization of the volatiles affords 1004.

### Example 1B

### Compound 1005

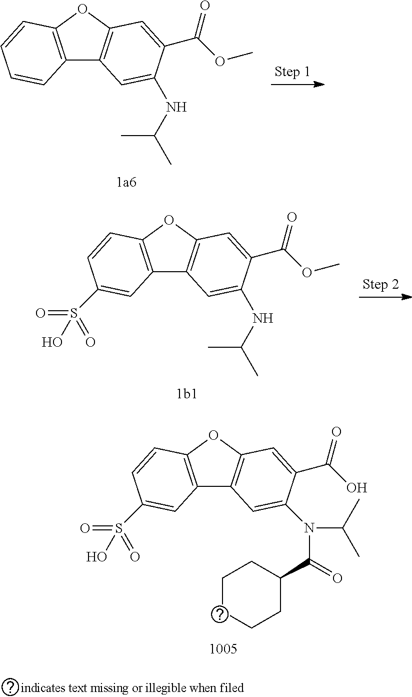

### Step 1;

Add 1a6 (30 mg, 0.11 mmol) to concentrated sulfuric acid (0.4 mL) and immerse in a sonication bath for about 30 min. Add water (4.6 mL) and filter the solids to afford 1b1.

### Step 2;

To a mixture of 1b1 (21 mg, 0.058 mmol) in anhydrous pyridine (2 mL) under an Ar atmosphere, add DMAP (1 mg, 0.007 mmol) and 1a8 (57 mg, 0.35 mmol). Heat to 60° C. for about 25 h. Evaporate the volatiles and dissolve the residue in DMSO (1 mL). Add 5 N NaOH (aqueous, 0.2 mL) and heat to 50° C. for about 1.6 h, then leave at RT for about 18 h. Add excess TFA until the pH is approximately <2. Purification affords 1005.

### Example 1C

### Compound 1012

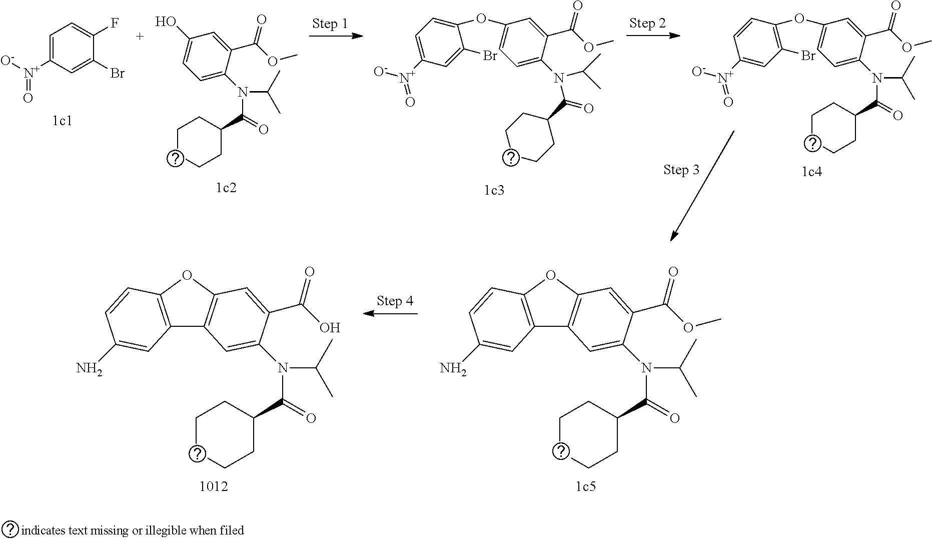

### Step 1:

Add to a mixture of 3-broma-4-fluoronitrobenzene (1c1, 245 mg, 1.12 mmol) and 1c2 (synthesis according to the procedure in WO 2007/087717) (264 mg, 0.79 mmol) in DMSO (2.8 mL), anhydrous potassium carbonate (154 mg, 1.11 mmol) and stir at 85° C. for about 2.5 h. Dilute the mixture with t-BME and wash successively with 1 N NaOH (aqueous), water and brine. Subsequently, dry with anhydrous MgSO₄, filter, and evaporate the volatiles. Purification by flash chromatography (EtOAc/Hexanes) provides 1c3,

### Step 2:

Reference: Keith Fagnou et al., J. Am. Chem. Soc. 2006, 128, 581-590.

Stir a mixture consisting of 1c3 (750 mg, 1.41 mmol), potassium carbonate (582 mg, 4.21 mmol), tricyclohexylphosphine tetrafluoroborate (108 mg, 0.28 mmol) and DMA (7 mL) at RT while bubbling Ar gas through the mixture for about 15 min. Add palladium(II)acetate (68 mg, 0.29 mmol) and immerse the reaction vessel into an oil bath preheated to 130° C., while maintaining a static Ar gas atmosphere. After about 30 min, cool the reaction to RT. Dilute the mixture with t-BME and wash with water. Dry with anhydrous MgSO₄, filter and evaporate the volatiles. Purification by flash chromatography (EtOAc/Hexanes) provides 1c4,

### Step 3:

Stir a mixture of 1c4 (156 mg, 0.34 mmol) and Pearlman's catalyst (60 mg) in MeOH (10 mL) under 1 atmosphere of hydrogen gas for about 2 h. Filter the solids and evaporate the volatiles to give 1c5.

### Step 4:

Using the protocol described in Example 1A, Step 7, 1c5 (20 mg, 0.47 mmol) is converted to 1012 as the TFA salt.

### Example 1D

### Compound 1006

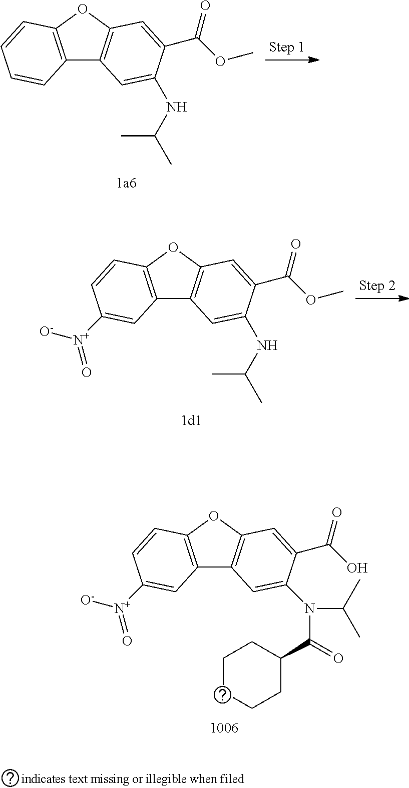

### Step 1:

To 1a6 (97 mg, 0.34 mmol) in cold concentrated sulfuric acid (2 mL), add KNO₃ (36 mg, 0.36 mmol) and stir the mixture at CPC for about 1 h. Dilute with EtOAc and water. To this biphasic mixture, add NaOH (solid) and Na₂CO₃ (solid) until the aqueous portion is basic. Separate the organic portion, dry it over Na₂SO₄, filter and evaporate. Purification by flash chromatography (EtOAc/Flexanes) provides 1d1.

### Step 2:

Using the protocol described in Example 1B, Step 2), 1d1 (14 mg, 0.044 mmol) is converted to 1006.

### Example 1E

### Compound 1007

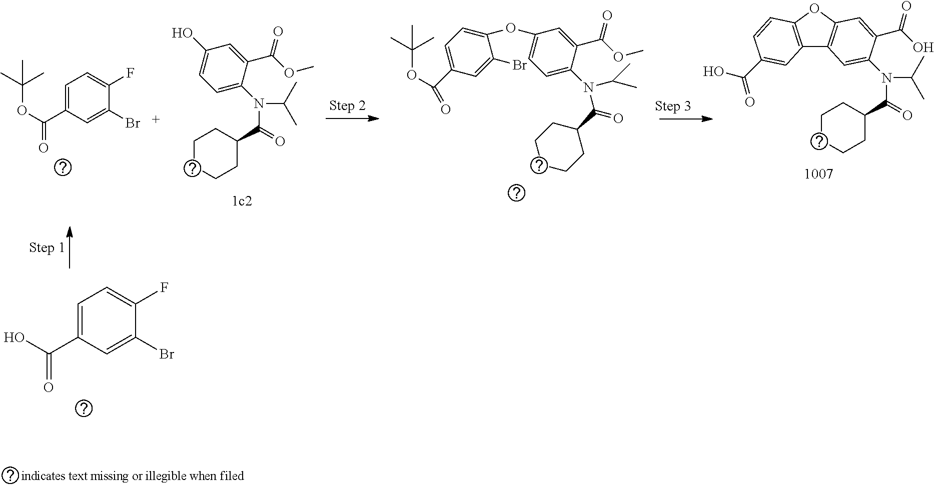

### Step 1:

Add carbonyl diimidazole (3.68 g, 22.7 mmol) to a mixture of 3-bromo-4-fluoro benzoic acid 1e1 (2.51 g, 11.5 mmol) in DMF (25 mL) and stir at RT for about 1 h. Cool the reaction to 0° C. and add t-BuOH (5.7 mL, 59.4 mmol). Then add DBU (1.9 mL, 12.6 mmol) dropwise. Allow the mixture to warm to RT and stir for about 21 h. Dilute the mixture with t-BME and wash successively with 10% citric acid (aqueous) and saturated NaHCO₃ (aqueous). Dry over MgSO₄, filter and evaporate the volatiles to obtain 1e2.

### Step 2:

Add anhydrous cesium carbonate (367 mg, 1.13 mmol) to a mixture of 1e2 (310 mg, 1.13 mmol) and 1c2 (255 mg, 0.76 mmol) in DMSO (4 mL) and stir at 50° C. for about 18 h. Dilute the mixture t-BME and wash successively with 10% citric acid (aqueous) and saturated NaHCO₃ (aqueous). Dry over Mg30₄, filter and evaporate the volatiles. Purification by flash chromatography (EtOAc/Hexanes) provides 1e3.

Step 3:
Reference: Keith Fagnou et al., J. Am. Chem. Soc. 2006, 128, 581-590.

Stir a mixture consisting of 1e3 (63 mg, 0.11 mmol), potassium carbonate (63 mg, 0.48 mmol), tricyclohexylphosphine tetrafluoroborate (6.5 mg, 0.017 mmol) and DMA (1 mL) at RT while bubbling Ar gas through the mixture for about 15 min. Add palladium(II)acetate (15 mg, 0.032 mmol) and immerse the reaction vessel into an oil bath preheated to 130° C., while maintaining a static Ar gas atmosphere. After about 15 h, cool the reaction to RT and dilute the mixture with EtOAc. Add AcOH (500 μL) and filter the solids. Collect the filtrate and wash with water and brine. Dry with anhydrous MgSO₄, filter, and evaporate the volatiles. Dissolve the residue in TFA (1 mL) and then evaporate. Dissolve the residue in DMSO (1 mL) and add 5 N NaOH (aqueous, 0.2 mL). Stir at 50° C. for about 2 h. Add excess TFA until the pH is approximately <2. Purification and lyophilization of the volatiles affords 1007.

### Example 1F

### Compound 1018

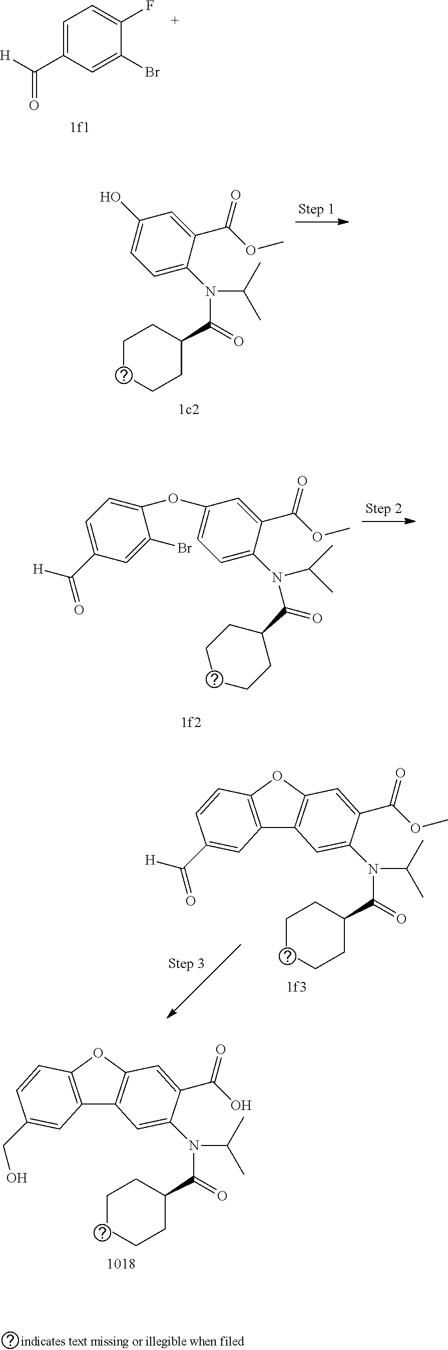

### Step 1:

In a fashion analogous to that for the production of 1c3 (Example 1C, Step 1), combine 3-bromo-4-fluoro benzaldehyde 1f1 (1.50 g, 7.39 mmol) and 1c2 (1.50 g, 4.5 mmol) in the presence of cesium carbonate (2.20 g, 6.75 mmol) to give 1f2.

### Step 2:

In a fashion analogous to that for the production of 1c4 (Example 1C, Step 2), combine 1f2 (2.15 g, 4.16 mmol) with potassium carbonate (1.73 g, 12.5 mmol), tricyclohexylphosphine tetrafluoroborate (320 mg, 0.84 mmol), DMA (100 mL) and palladiurn(II)acetate (200 mg, 0.87 mmol) at 130° C. for about 90 min to give 1f3.

### Step 3:

Dissolve 1f3 (14 mg, 0.033 mmol) into a mixture of MeOH (0.2 mL) and THF (0.2 mL) at RT. Add NaBH₄ (10 mg, 0.26 mmol) and stir for about 1 h. Evaporate the volatiles and dissolve the residue in DMSO (1.5 mL). Add 5 N NaOH (aqueous, 0.2 mL) and heat to 45° C. for about 45 min. Add excess AcOH. Purification and lyophilization of the volatiles affords 1018.

### Example 1G

### Compounds 1054, 1055 and 1056

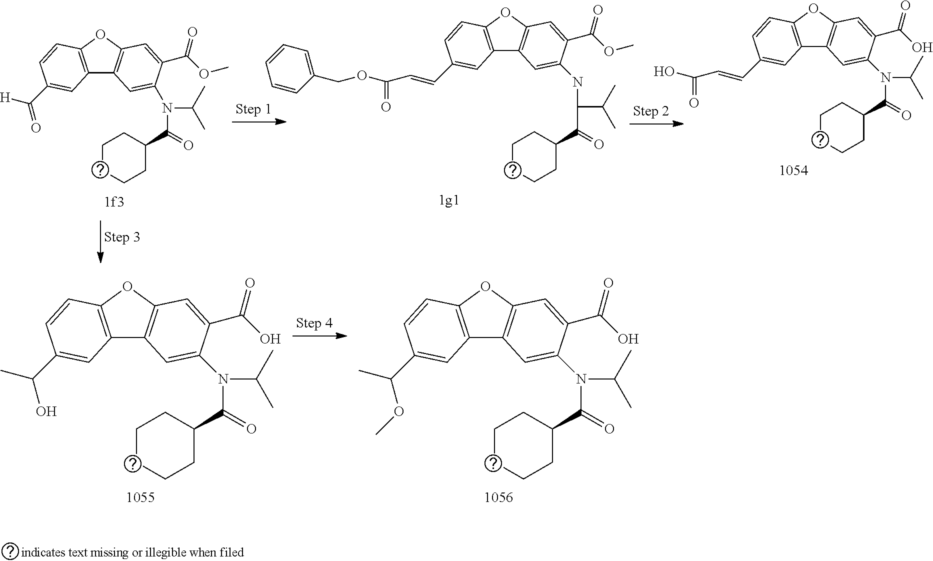

### Step 1:

Dissolve 1f3 (1.00 g, 2.29 mmol) in anhydrous DCM (100 mL) and add benzyl (triphenylphosphoranylidene)acetate (1.07 g, 2.54 mmol). Stir this mixture for about 2 days at RT. Evaporate the volatiles and purify with flash chromatography (EtOAc/Hexanes) to give 191.

### Step 2:

Add 1 N NaOH (aqueous, 0.3 mL) to a mixture of (30 mg, 0.088 mmol) in DMSO (1 mL) and heat to 50° C. After about 2 h at RT, add excess TFA to adjust the pH to approximately <2. Purification and lyophilization of the volatiles affords 1054,

### Step 3:

Dissolve 1f3 (100 g, 0.23 mmol) in anhydrous THF (2 mL) and cool to −78° C. Add a solution of 1.4 M CH₃Li in Et₂O (250 μL, 0.34 mmol) dropwise. Then add water (100 μL) and evaporate the volatiles. Dissolve the residue in DMSO (3 mL) and add 5 N NaOH (aqueous, 0.5 mL). Stir at RT for about 1 h. Add excess AcOH until the pH is approximately <4. Purification and lyophilization of the volatiles affords 1055.

### Step 4:

Dissolve 1055 (37 mg, 0.085 mmol) in THF (1 mL) and add a 0.7 M diazomethane/t-BME solution (2.5 mL). Evaporate the volatiles and dissolve the residue in anhydrous DMF (1 mL). Add MeI (27 μL, 0.42 mmol), followed by 60% NaH/mineral oil (6.8 mg, 0.17 mmol). Stir at RT for about 2 h. Add DMSO (1 mL) and 1 N NaOH (aqueous, 0.5 mL) and stir at RT for about 2 h. Add excess AcOH until the pH is approximately <4. Purification and lyophilization of the volatiles affords 1056.

### Example 1H

### Compounds 1057, 1058 and 1140

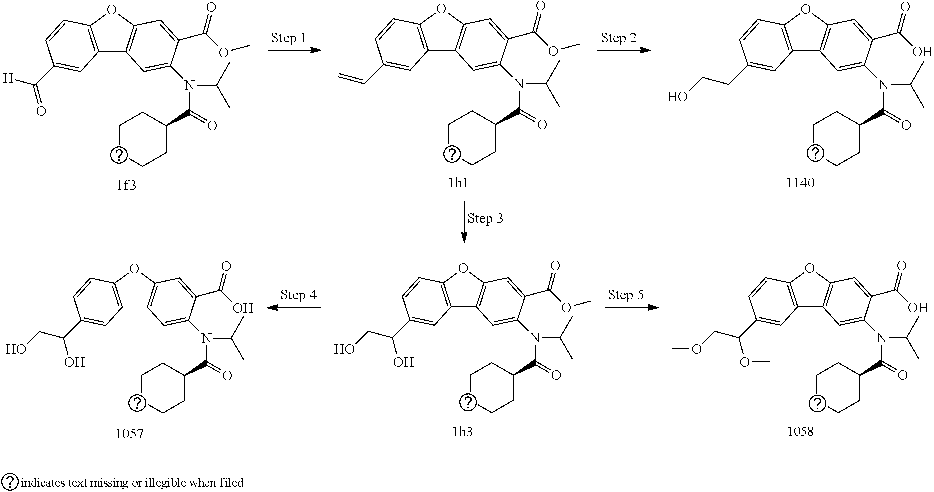

### Step 1:

Add solid potassium t-butoxide (112 mg, 1.00 mmol) to a suspension of methyltriphenylphosphonium bromide (357 mg, 1.00 mmol) in THF (10 mL) at −78° C. Warm the mixture slowly to 0° C. over about a 1 h period. To the suspension, add 1f3 (218 mg 0.500 mmol) dissolved in THF (5 mL). Stir for about 1 h. Add AcOH (50 μL) and evaporate the volatiles. Purification of the residue by flash chromatography (EtOAc/Hexanes) affords 1 h1.

### Step 2:

Add a borane-dimethyl sulfide complex (46 μL, 0.46 mmol) to a solution of 1h1 (100 mg, 0.23 mmol) in THF (5 mL) at −78° C. Allow the mixture to warm slowly to RT. Slowly add 30% H₂O₂ (aqueous, 0.5 mL), followed by 1 N NaOH (aqueous, 0.5 mL). Stir at RT for about 24 h. Evaporate the volatiles and acidify with AcOH and DMSO. Purification and lyophilization of the volatiles affords 1140.

### Step 3:

Add to a solution of 1h1 (100 mg, 023 mmol) in acetone (5 mL) at RT, 60% aqueous N-methylmorpholine-N-oxide (80 μL, 0.46 mmol), followed by the addition of a solution of 2.5% w/w OsO₄ in t-BuOH (290 μL, 0.023 mmol). Stir for about 2 h, then evaporate the volatiles. Add toluene (4 mL) and evaporate. Purification by flash chromatography (EtOAc/Hexanes) gives 1h3.

### Step 4.

Using the protocol described in Example 1A, Step 7, 1h3 (30 mg, 0.064 mmol) is convereted to 1057.

### Step 5:

Dissolve 1h3 (60 mg, 0.128 mmol) in anhydrous DMF (2 mL) and add MeI (65 μL, 1.03 mmol), followed by 60% NaH/mineral oil (28 mg, 010 mmol). Stir at RT for about 3 h, then add DMSO (2 mL). Add 1 N NaOH (aqueous, 0.4 mL) and stir at RT for about 18 h. Add excess AcOH until the pH is approximately <4. Purification and lyophilization of the volatiles affords 1058.

### Example 11

### Compounds 1136 and 1139

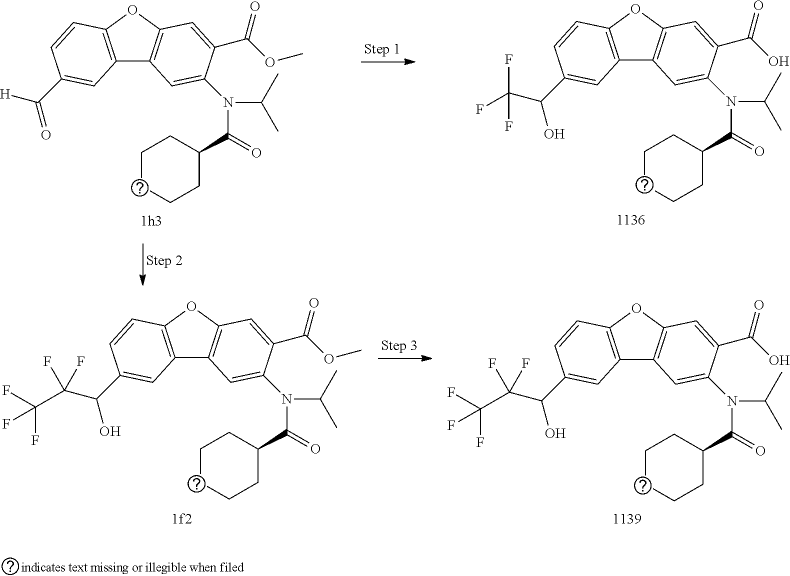

### Step 1:

Following the procedure outlined in Tetrahedron, 2000, 56(2), pp 275-283, dissolve KH (40 mg, 1.00 mmol) in anhydrous DMSO (2 mL). Add this solution to a mixture of gaseous CF₃H (100 mg, 1.43 mmol) in anhydrous DMF (3 mL) at −40 to −50° C. and stir for about 40 min. Add 1f3 (144 mg, 0.33 mmol) in DMF (2 mL). Stir the mixture at −50° C. for about 2 h, then at RT for about 3 days. Dilute with water, extract with EtOAc, dry the organic portion with Na₂SO₄, filter and evaporate. Dissolve the residue in DMSO. Purification and lyophilization of the volatiles affords 1136.

Step 2: Following the procedure outlined in JOC, 1987, 52(12), pp 2481-2490, to a flask containing gaseous CF₃CF₂I (635 mg, 2.58 mmol) and Et₂O (10 mL), add 1f3 (150 mg, 0.34 mmol) in a solution of Et₂O (10 mL) and cool to −78° C. Add 1.4 M

CH₃Li LiBr complex in Et₂O (445 μL, 0.62 mmol) portionwise. After about 10-15 mins, add 10% citric acid (aqueous, 4 mL) and then allow the mixture to warm to RT. Separate the layers and pass the organic portion through a small pad of Extrelut®. Collect the filtrate and evaporate. Purification of the residue by flash chromatography (EtOAc/Hexanes) affords 1i2 as an impure solid.

### Step 3:

Using the protocol described in Example 1A, Step 7, 1i2 (35 mg, 0.063 mmol) is converted to 1139.

### Example 1J

### Compounds 1141 and 1142

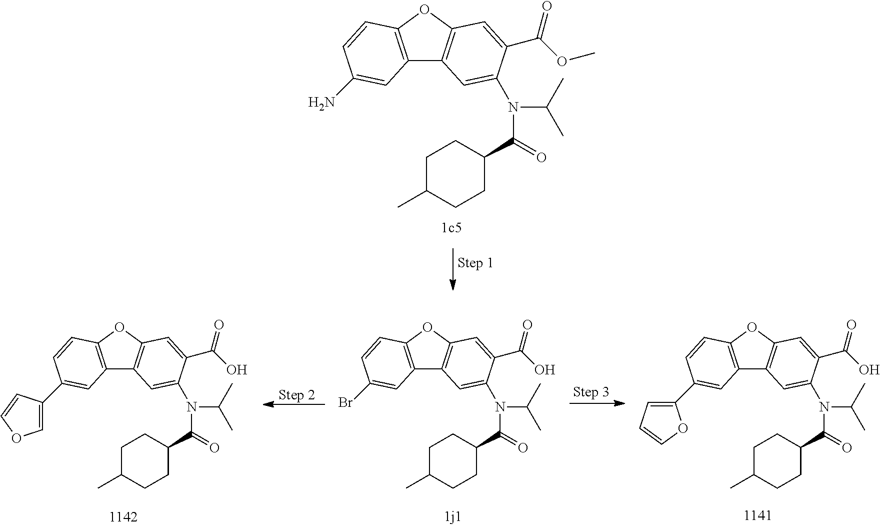

### Step 1:

Dissolve 1c5 (274 mg, 0.55 mmol) in MeCN (2.5 mL) and add TFA (5 mL). Cool the mixture to 0° C. Add aqueous sodium nitrite (67 mg/0.5 mL, 0.97 mmol) dropwise and stir for about 1 h. Add cupric bromide (434 mg, 1.95 mmol) in a mixture of MeCN (2.5 mL) and water (2 mL). Add solid cuprous bromide (279 mg, 1.95 mmol) as a solid and stir for about 1 h. Evaporate the volatiles, dilute with t-BME, wash with water, dry over MgSO₄, filter and evaporate. Purdication of the residue by flash chromatography (EtOAc/Hexanes) affords 1j1.

### Step 2:

To a mixture consisting of 1j1 (35 mg, 0.072 mmol), 3-furanboronic acid (20 mg, 0.18 mmol), toluene (1.5 mL), EtOH (1.5 mL), water (1 mL), LiCl (9 mg, 0.22 mmol), and Na₂CO₃ (27 mg, 0.25 mmol) under an Ar gas atmosphere, add (Ph₃P)₄Pd (8 mg, 0.007 mmol) and heat to 90° C. for about 20 h. Evaporate the volatiles and then add DMSO (2 mL) and 5 N NaOH (aqueous, 0.3 mL). Stir the mixture at 50° C. for about 4 h and then acidify to approximately pH<2 with TFA. Purification and lyophilization of the volatiles affords 1142.

### Step 3:

Using the protocol described in Example 1J, step 2, 1j1 (35 mg, 0.072 mmol) with 2-furanboronic acid (24 mg, 0.22 mmol) is converted to 1141.

### Example 1K

### Compound 1010

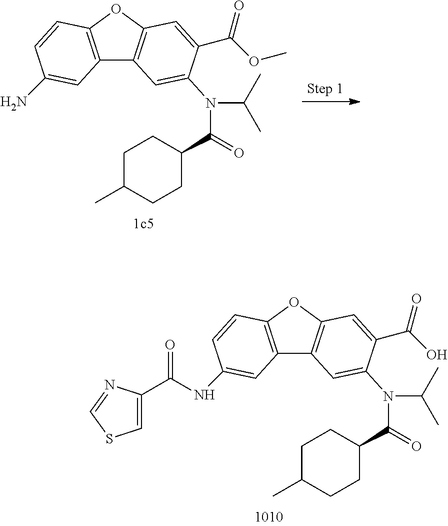

### Step 1:

Stir a mixture of 1c5 (28 mg, 0.066 mmol), TEA (46 μL, 0.33 mmol), TBTU (27 mg, 0.084 mmol), 1,3-thiazole-4-carboxylic acid (18 mg, 0.13 mmol) in DMSO (1.5 mL) for about 18 h. Add 5 N NaOH (aqueous, 0.2 mL) and heat to 45° C. for about 1 h. Acidify to approximately pH<2 with TFA, purify and lyophilize the volatiles to afford 1010.

### Example 1L

### Compounds 1031 and 1059

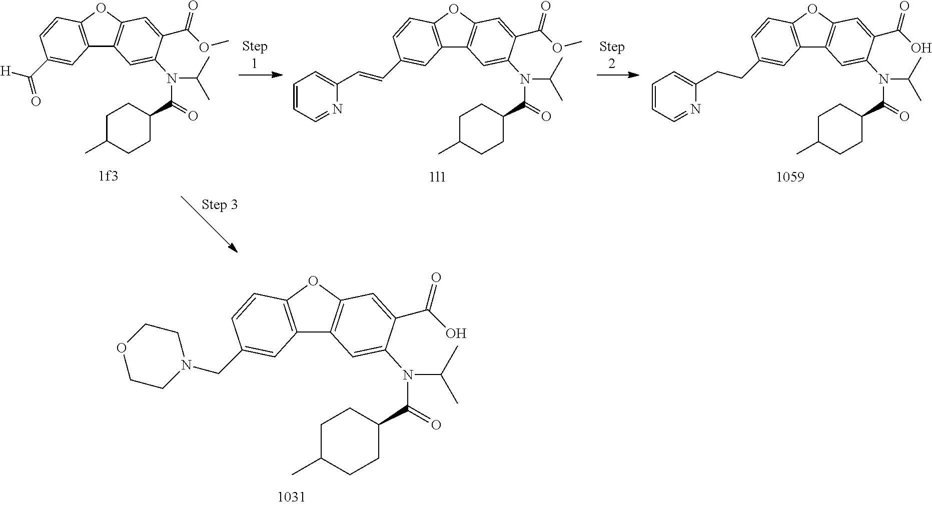

### Step 1;

Using triphenyl(2-pyridylmethyl)phosphonium chloride hydrochloride as reagent and the protocol described in Example 1H, Step 1, 1f3 (218 mg, 0.500 mmol) is converted to 1l1.

### Step 2:

Expose 1l1 (45 mg, 0.088 mmol) in MeOH (15 mL) containing Pearlman's catalyst to a hydrogen gas atmosphere (1 atm) for about 3 h. Filter the solids and evaporate the volatiles. Add DMSO (1 mL) and 5 N NaOH (aqueous, 0.2 mL) and stir at RT for about 18 h. Acidify to approximately pH<2 with TFA. Purification and lyophilization of the volatiles affords 1059 as the TFA adduct.

### Step 3:

Stir 1f3 (25 mg, 0.57 mmol), morpholine (30 μL), MeOH (500 μL) and AcOH (30 μL) at 50° C. for about 1 h. Cool to RT and add sodium cyanoborohydride (5.0 mg, 0.079 mmol). Stir for about 18 h. Add the mixture to 10% Na₂CO₃ (aqueous, 10 mL), whereupon a precipitate forms. The solids are collected by filtration, then are dissolved in DMSO (1 mL) and 2.5 N NaOH (aqueous, 0.1 mL). Stir the mixture at RT for about 18 h. Acidify until the pH is approximately <2 with TFA. Purification and lyophilization of the volatiles affords 1031 as the TFA salt.

### Example 1M

### Compound 1109

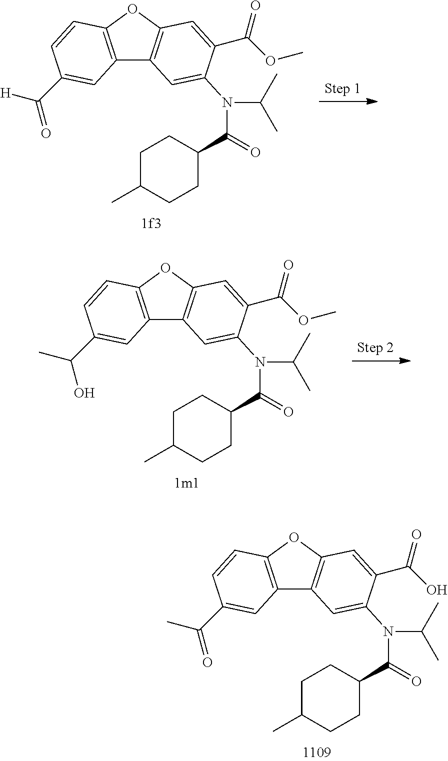

### Step 1:

Add a 1.4 M solution of CH₃Li/Et₂O (300 μL, 0.42 mmol) to 1f3 (100 mg, 0.23 mmol) in anhydrous THF (2 mL) at −78° C. and stir for about 30 min. Quench the reaction with AcOH (25 μL) and evaporate. Purification of the residue with flash chromatography (EtOAc/Hexanes) gives crude 1m1 which is used as such in the subsequent reaction.

### Step 2:

To crude 1m1 (70 mg) in THF (2 mL), add manganese dioxide (159 mg, 1.55 mmol) at RT and stir for about 3 h. Filter the solids and evaporate the volatiles. Dissolve the residue in DMSO (2 mL) and add 5 N NaOH (aqueous, 0.3 mL). Stir at RT for about 1 h, then add excess TFA to adjust the pH to approximately <2. Purification and lyophilization of the volatiles affords 1109.

### Example 1N

### Compound 1050

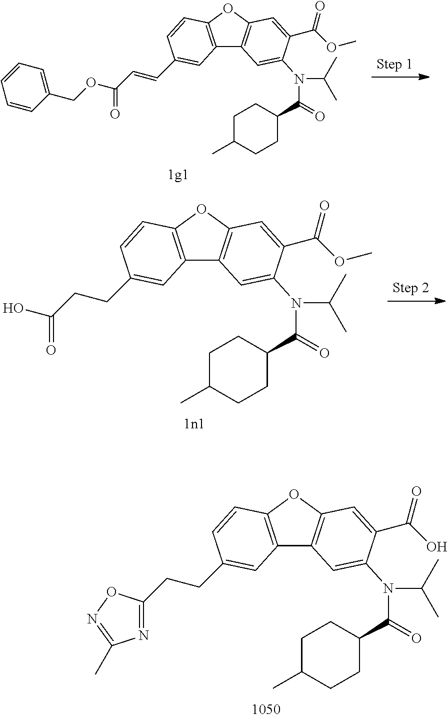

### Step 1:

Expose 1g1 (235 mg, 0.41 mmol) in MeOH (30 mL) containing Pearlman's catalyst (100 mg) to a hydrogen gas atmosphere (1 atm) for about 14 h. Filter the solids and evaporate the volatiles to give 1n1.

### Step 2:

Stir a mixture consisting of 1n1 (50 mg, 0.10 mmol), DIPEA (94 μL, 0.54 mmol), acetamide oxime (9.5 mg, 0.13 mmol), TBTU (41 mg, 0.13 mmol) and DMF (2 mL) at RT for 18 h. Dilute with t-BME, wash with water, then filter the organic portion through a pad of EXTRELUT®. Collect the filtrate and evaporate. Dilute the residue in THF (2 mL) and add 1 M TBAF/THF (100 μL, 0.10 mmol). Stir the mixture at RT for about 2 h. Evaporate the volatiles and dissolve the residue in DMSO (1 mL). Add 5 N NaOH (aqueous, 0.3 mL) and stir at RT for about 2 h. Add excess AcOH until the pH is approximately <4. Purification and lyophilization of the volatiles affords 1050.

### Example 1O

### Compounds 1173, 1174 and 1176

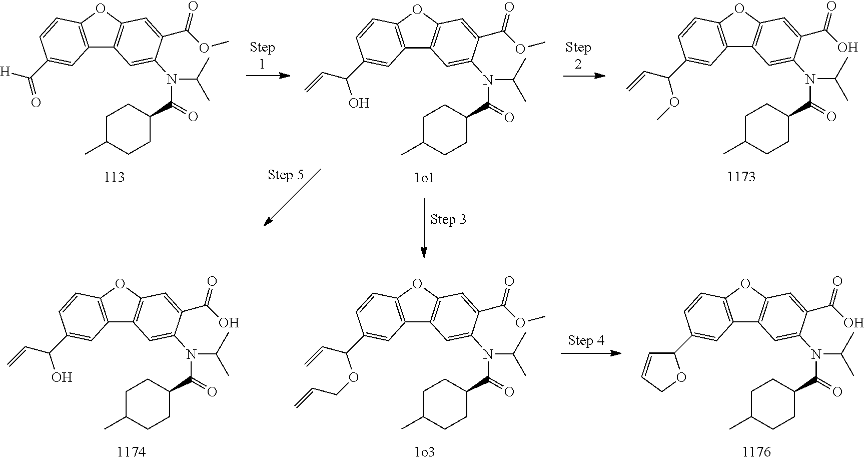

### Step 1:

Add 1.6 M vinylmagnesium bromide/THF (400 μL, 0.63 mmol) to 1f3 (250 mg, 0.54 mmol) in THF (5 mL) at 0° C. and stir for about 1 h. Dilute with saturated NH₄Cl (aqueous) and EtOAc. Separate the layers, dry the organic portion with MgSO₄, filter and evaporate. Purification of the residue with flash chromatography (EtOAc/Hexanes) provides 1o1.

### Step 2:

Add iodomethane (200 μL) and 60% w/w NaH/mineral oil (9 mg, 0.22 mmol) to 1o1 (50 mg, 0.11 mmol) in THF (1 mL) at 0° C. Warm the mixture to 40° C. for about 18 h, then cool the mixture to RT. Dilute with MeOH, then add 1 N NaOH (aqueous). Stir at RT for about 24 h. Purification and lyophilization of the volatiles affords 1173.

### Step 3:

Add allyl bromide (380 μL, 0.38 mmol) and 95% NaH (11 mg, 0.46 mmol) to 1o1 (135 mg, 0.29 mmol) in THF (3 mL) at 0° C. Warm the mixture to RT and stir for about 14 h. Dilute with saturated NH₄Cl (aqueous) and EtOAc and separate the layers. Dry the organic portion with MgSO₄, filter and evaporate. Purification of the residue with flash chromatography (EtOAc/Hexanes) provides 1o3.

### Step 4:

Dissolve 1o3 (60 mg, 0.12 mmol) in degassed toluene (30 mL) and add Hoveyda-Grubb's 2^{nd} generation catalyst (7 mg, 0.008 mmol). Heat the mixture in a pre-warmed oil bath set at 80° C. for about 30 min then cool to RT. Add silica gel and filter. Evaporate the volatiles and dilute the residue with EtOAc. Filter, evaporate and take-up the residue in THF/MeOH and 1 N NaOH (aqueous). Stir this mixture at RT for about 18 h. Purification and lyophilization of the volatiles affords 1176.

### Step 5:

Using the protocol described in Example 1A, Step 7, 1o1 (50 mg, 0.11 mmol) is converted to 1174.

### Example 1P

### Compound 1175

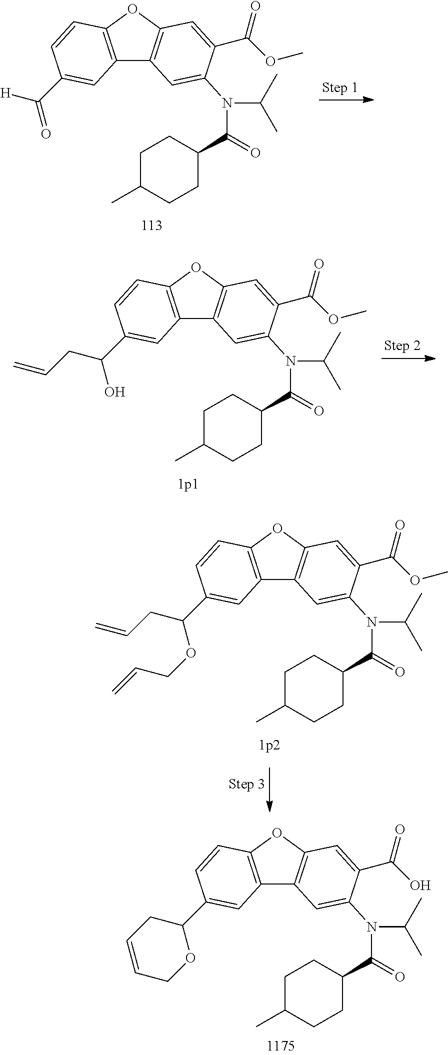

### Step 1:

Add 2 M allylmagnesium bromide/THF (44 μL, 0.88 mmol) slowly to 1f3 (350 mg, 0.80 mmol) in THF (7 mL) at 48° C., and then allow to warm to 0° C. Dilute with saturated NH₄Cl (aqueous) and EtOAc and separate the layers. Dry the organic portion with MgSO₄, flier and concentrate. Purification of the residue with flash chromatography (EtOAc/Hexanes) affords 1p1.

### Step 2:

Using the protocol described in Example 1O, Step 3, 1p1 (53 mg, 0.11 mmol) is converted to 1p2.

### Step 3:

Using the protocol described in Example 1O, Step 4, 1p2 (23 mg, 0.044 mmol) is converted to 1175.

### Example 1Q

### Compound 2002

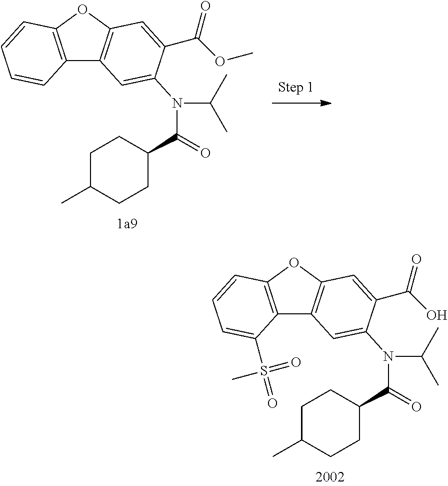

### Step 1:

Following the procedure outlined in Chemistry Letters, 1988, pp 395-398, add solid methanesulfonic anhydride (426 mg, 2.45 mmol) and triflic acid (200 μL, 2.26 mmol) to a mixture of solid 1a9 (100 mg, 0.25 mmol), and heat to approximately 80° C. for about 1 h. Add this mixture to water (20 mL), then extract with EtOAc (2×20 mL). Combine the organic portions and evaporate. Dissolve the residue in DMSO (4 mL) and add 5 N NaOH (aqueous, 0.5 mL). Stir at RT for about 18 h then add excess TFA until the pH is approximately <2. Purification and lyophilization of the volatiles affords 2002.

### Example 2A

### Compound 1025

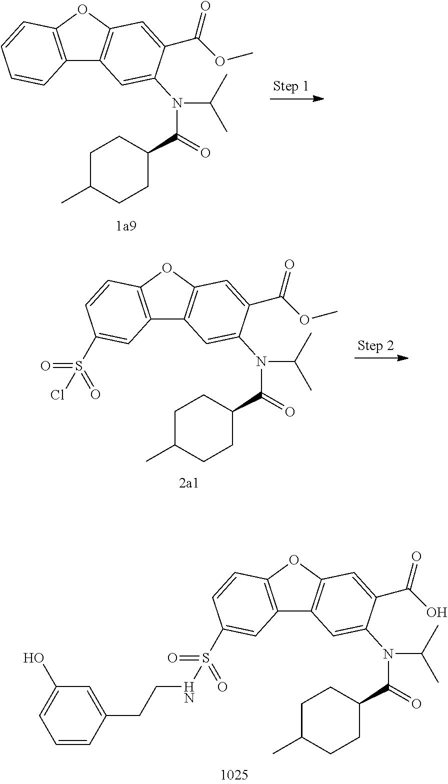

### Step 1:

Add chlorosulfonic acid (400 μL, 5.99 mmol) to 1a9 (250 mg, 0.61 mmol) in DCM (10 mL), and stir at RT for about 18 h. Dilute with t-BME, and wash with water and saturated NaHCO₃ (aqueous). Dry over MgSO₄, filter and concentrate. Purification of the residue with flash chromatography (EtOAc/Hexanes) gives 2a1.

### Step 2:

Add to 3-hydroxyphenethylamine hydrochloride salt (21 mg, 0.12 mmol) in DCM (2 mL), TEA (100 μL, 0.72 mmol) and 2a1 (30 mg, 0.059 mmol) in THF (1 mL). Stir at RT for about 2 h, then evaporate the volatiles. Dissolve the residue in DMSO (1 mL). Add 1 N NaOH (aqueous, 0.3 mL) and stir at RT for about 18 h. Add excess TFA until the pH is approximately <2. Purification and lyophilization of the volatiles affords 1025.

### Example 3A

### Compound 1024

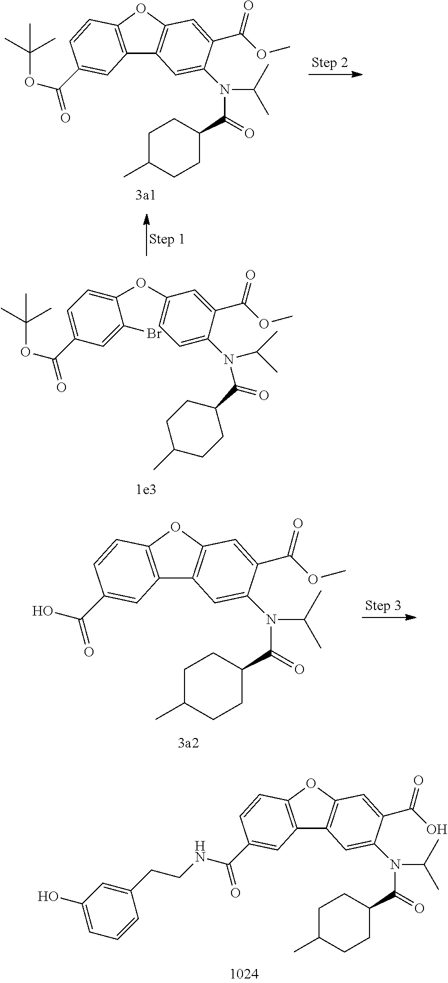

### Step 1:

Combine 1e3 (573 g, 0.97 mmol) with potassium carbonate (404 g, 2.92 mmol), tricyclohexylphosphine tetrafluoroborate (54 mg, 0.15 mmol), DMA (10 mL) and palladium(II)acetate (22 mg, 0.097 mmol) at 130° C. for about 90 min. Dilute with t-BME and add excess AcOH (0.4 mL). Wash with water and dry over MgSO₄, filter and concentrate. Dissolve the residue in t-BME (5 mL) and add a 0.7 M diazomethane solution in t-BME (10 mL). Evaporation of the volatiles and purification of the residue with flash chromatography (EtOAc/Hexanes) affords 3a1.

### Step 2:

Dissolve 3a1 (445 mg, 0.88 mmol) in TFA (4 mL) and stir at RT for about 2 h. Evaporate the volatiles then co-evaporate with toluene. Triturate with hexanes to give 3a2.

### Step 3:

Stir a mixture of 3a2 (30 mg, 0.066 mmol). TEA (92 μL, 0.66 mmol), TBTU (43 mg, 0.13 mmol) and 3-hydroxyphenethylamine hydrochloride salt (23 mg, 0.13 mmol) in DMSO (1 mL) for about 2 h. Add 5 N NaOH (aqueous, 0.3 mL) and stir at RT for about 18 h. Acidify with TFA until the pH is approximately <2. Purification and lyophilization of the volatiles affords 1024.

### Example 3B

### Compound 1052

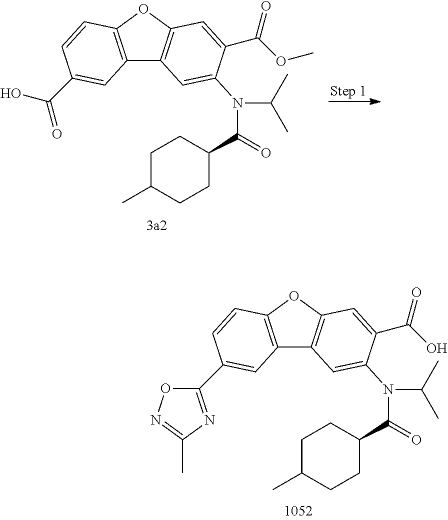

### Step 1:

Stir a mixture consisting of 3a2 (65 mg, 0.14 mmol). DIPEA (125 μL, 0.72 mmol), acetamide oxime (13 mg, 0.18 mmol), TBTU (55 mg, 0.17 mmol) and THF (2 mL) at RT for about 3 h. Add additional acetamide oxime (26 mg) and TBTU (275 mg), then stir for about 18 h. Dilute with t-BME, wash with water, then filter the organic portion through a pad of EXTRELUT®. Collect the filtrate and evaporate. Dilute the residue in THF (2 mL), add 1 M TBAF/THF (100 μL, 0.10 mmol) and stir at RT for about 2 h. Evaporate the volatiles and dissolve the residue in DMSO (1 mL). Add 5 N NaOH (aqueous, 0.3 mL) and stir at RT for about 2 h. Add excess AcOH until the pH is approximately <4. Purification and lyophilization of the volatiles affords 1052.

### Example 4A

### Compounds 1060, 1063 and 1066

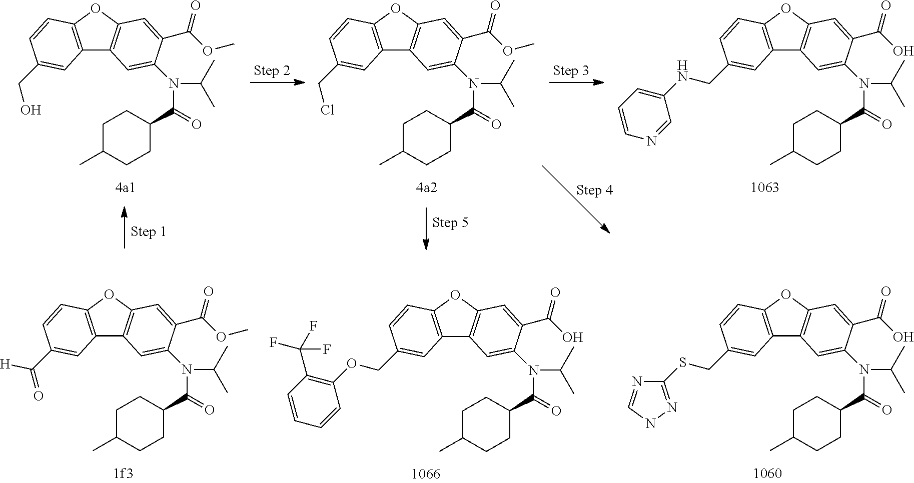

### Step 1:

Stir a mixture of 1f3 (3.00 g, 6.89 mmol), MeOH (150 mL) and sodium borohydride (300 mg, 7.93 mmol) at RT for about 1 h. Quench the reaction with excess 4 N HCl (aqueous) and stir for about 30 min. Evaporate the volatiles and dissolve the residue in EtOAc. Wash successively with water, saturated NaHCO₃ (aqueous) and brine, dry over MgSO₄, filter and concentrate to provide crude 4a1.

### Step 2:

Add to a mixture of crude 4a1 (3.00 g, 6.86 mmol) in anhydrous DCM (150 mL) and DMF (0.3 mL) at RT, thionyl chloride (1.50 mL, 21.0 mmol) and stir for about 20 min. Evaporate the volatiles and purify with flash chromatography (EtOAc/Hexanes) to provide partially purified material. Precipitate the product with a combination of EtOAc, Et₂O and hexanes and filter the solid to give 4a2.

### Step 3:

Add to a mixture of Cs₂CO₃ (19 mg, 0.058 mmol), KI (2.5 mg, 0.015 mmol), 3-aminopyridine (5.0 mg, 0.053 mmol) and MgSO₄ (20 mg), a solution of 4a2 (20 mg, 0.042 mmol) in DMF (500 μL). Stir at 70° C. for about 4 h then at RT overnight. Filter the mixture and then wash the filter with DMSO (500 μL). Combine the filtrate and washings and add 5 N NaOH (aqueous, 100 μL). Stir at RT for about 3 h. Acidify with excess AcOH. Purification and lyophilization of the volatiles affords 1063 as the TFA salt.

### Step 4:

Using the protocol described in Example 4A, Step 3, 4a2 (35 mg, 0.072 mmol) with 3-mercepto-1,2,4-triazole (5.4 mg, 0.053 mmol) is converted to 1060.

### Step 5:

Using the protocol described in Example 4A, Step 3, 4a2 (35 mg, 0.072 mmol), with 2-hydroxybenzotrifluoride (8.6 mg, 0.053 mmol) is converted to 1066.

### Example 4B

### Compound 1038

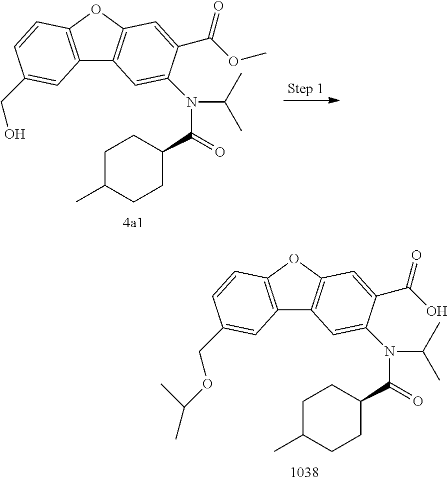

### Step 1:

Add compound 4a1 (20 mg, 0.045 mmol) to a mixture of 60% w/w NaH/mineral oil (6.0 mg, 0.15 mmol) in DMF (0.5 mL) and stir at RT for about 15-30 mins. Add 2-iodopropane (116 μL, 1.18 mmol) in portions and stir for about 3 days. Dilute with DMSO (1 mL) and acidify with excess AcOH. Purification and lyophilization of the volatiles affords 1038.

### Example 4C

### Compounds 1040, 1053 and 1129

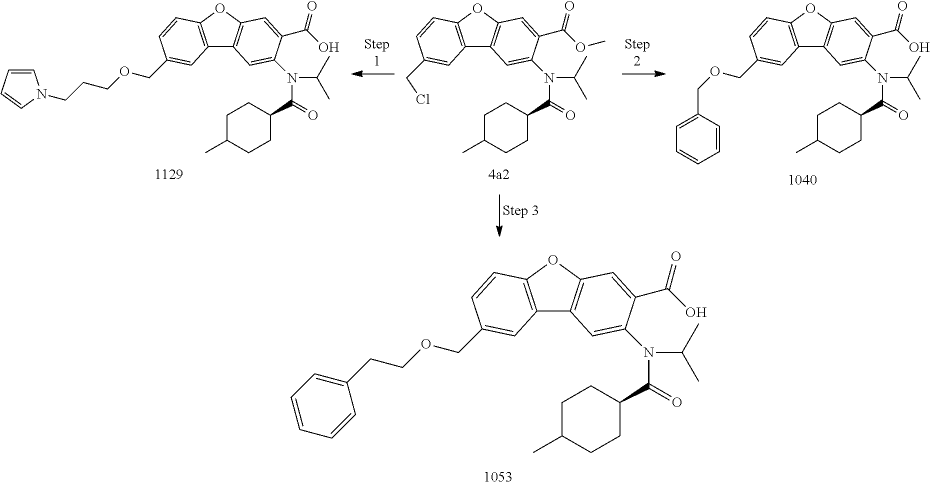

### Step 1;

Add to a mixture of KI (14 mg, 0.084 mmol) and 1-(3-hydroxypropyl)pyrrole (12.5 mg, 0.10 mmol), 60% w/w NaH/mineral oil (5.0 mg, 0.12 mmol) in DMSO (0.5 mL) (premix at 80° C. for about 1 h, then cool to RT). Stir for about 10 min, then add 4a2 (20 mg, 0.044 mmol) in DMSO (0.5 mL). Stir at RT for about 72 h. Add 5 N NaOH (aqueous, 100 μL) and stir for about 2 h. Dilute to a 1.5 mL volume with AcOH. Purification and lyophilization of the volatiles affords 1129.

### Step 2;

Add to a mixture of 60% w/w NaH/mineral oil (4.0 mg, 0.10 mmol) in DMF (0.5 mL), compound 4a2 (20 mg, 0.043 mmol) followed by benzyl alcohol (10 μL, 0.096 mmol). Stir at RT for about 1 h, then dilute with DMSO (1 mL) and acidify with excess AcOH. Purification and lyophilization of the volatiles affords 1040.

### Step 3:

Add potassium t-butoxide (15 mg, 0.13 mmol) to a mixture of KI (22 mg, 0.13 mmol), DMF (1 mL) and 2-phenylethanol (20 μL, 0.16 mmol) at RT. Stir for about 5-10 min. Add 4a2 (30 mg, 0.066 mmol) dissolved in DMF (1 mL) and stir for about 3 h. Add an additional portion of potassium t-butoxide (15 mg, 0.13 mmol) and stir for about 18 h. Acidify with excess TFA. Purification and lyophilization of the volatiles affords 1053.

### Example 5A

### Compound 1166

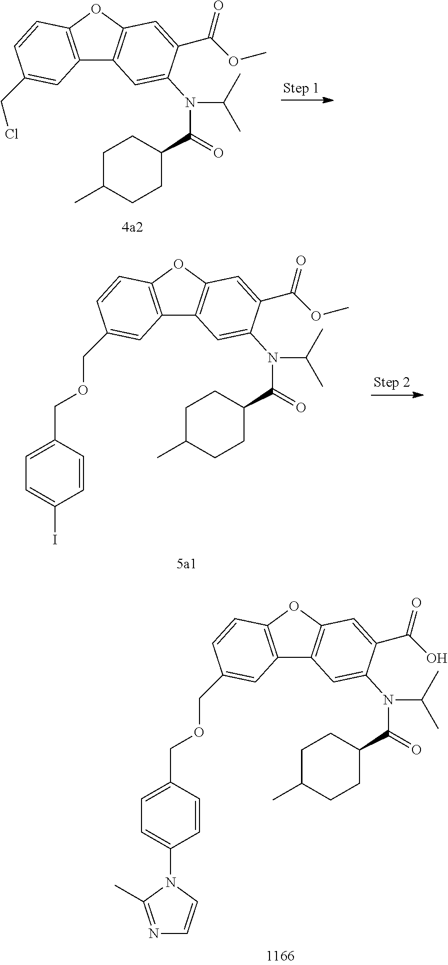

### Step 1:

Deposit 60% w/w NaH/mineral oil (100 mg, 2.50 mmol) into a 100 mL round bottom flask and wash with hexanes (20 mL). Add anhydrous DMSO (10 mL) and heat to 80° C. for about 1 h, then cool. Add 4-iodo benzyl alcohol (246 mg, 1.05 mmol) and KI (280 mg, 1.69 mmol) and stir for about 10 min. Add 4a2 (400 mg, 0.88 mmol) and stir for about 18 h at RT. Add the mixture to 1 N HCl (aqueous, 200 mL), whereupon a precipitate forms. Filter the solids. Dissolve the solids in EtOAc and wash with brine, dry over MgSO₄, filter and concentrate. Dissolve the residue in MeCN (6 mL) and add DBU (116 μL, 0.77 mmol) and iodomethane (200 μL, 3.22 mmol) portionwise over about 8 h. Stir for about 18 h at RT. Evaporate the volatiles and dissolve the residue in EtOAc. Wash successively with brine, saturated NaHCO₃ (aqueous) and 1 N HCl (aqueous) and brine. Dry over MgSO₄, filter and concentrate. Purification of the residue by flash chromatography (EtOAc/Hexanes) affords 5a1.

### Step 2:

Reference: Immaculada Dinarés et al., Eur. J. Org. Chem. 2005, 1637-1643.

Add a solution of 5a1 (19 mg, 0.029 mmol) in DMF (500 μL) to a mixture of Cs₂CO₃ (24 mg, 0.074 mmol), cuprous iodide (1.8 mg, 0.009 mmol) and 2-methylimidazole (3.0 mg, 0.036 mmol). Place under an atmosphere of Ar as and add trans-1,2-bis(methylamino)cyclohexane (3.0 mg, 0.021 mmol). Stir at 100° C. for about 36 h, cool to RT, then add 5 N NaOH (aqueous, 72 μL). Stir at 55° C. for about 2 h. Dilute to a 1.5 mL volume with AcOH. Purification and lyophilization of the volatiles affords 1166 as the TFA salt.

### Example 5B

### Compounds 1137 and 1138

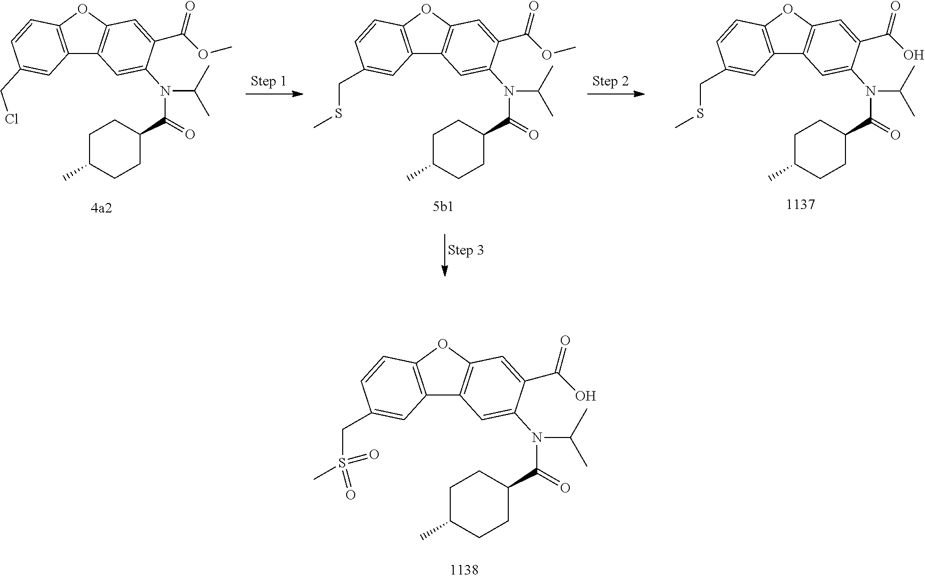

### Step 1:

To 4a2 (75 mg, 0.16 mmol) in THF (2 ml) at RT, add sodium methylthiolate (14 mg, 0.20 mmol) and stir for about 3 h. Add DMF (2 mL) and heat to 70° C. for about 16 h. Add a further portion of sodium methylthiolate (10 mg, 0.14 mmol) and stir for about 3 h. Evaporate the volatiles and dilute with t-BME. Wash with water, dry over MgSO₄, filter and concentrate. Purification of the residue by flash chromatography EtOAc/Hexanes) affords 5b1.

### Step 2:

Using the protocol described in Example 1A, Step 7, 5b1 (15 mg, 0.032 mmol) is converted to 1137.

### Step 3:

Add Oxone® (150 mg, 0.24 mmol) to a mixture of 5b1 (28 mg, 0.060 mmol), acetone (6 mL) and water (2 mL) at RT. Stir for about 4 h. Evaporate the acetone, co-evaporate with EtOH (3 mL). Add DMSO (3 mL) and 5 N NaOH (aqueous, 1 mL) to the residues and stir at RT for about 30 min. Acidify with excess AcOH. Purification and lyophilization of the volatiles affords 1138.

### Example 6A

### Compounds 1180 and 1190

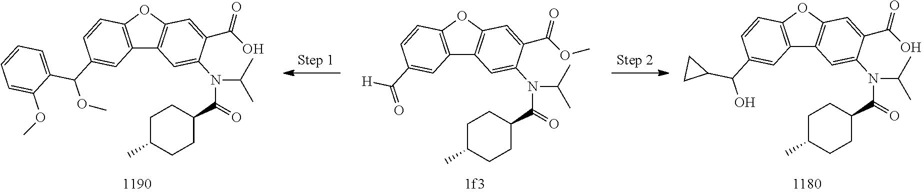

### Step 1:

Add 1 M/THF 2-methoxyphenyl magnesium bromide (690 μL, 0.67 mmol) to 1d3 (100 mg, 0.23 mmol) in THF (5 mL) at 0° C., and stir for about 1 h. Add 1 N HCl (aqueous, 100 μL) and dilute with t-BME. Wash with 1 N HCl (aqueous), dry over Na₂SO₄, filter and concentrate. Dissolve the residue in anhydrous DMF (2 mL) and add methyl iodide (73 μL, 1.15 mmol) followed by 60% NaH/mineral oil (48 mg, 1.15 mmol). Stir at RT for about 1 h, then add water (100 μL), DMSO (2 mL), and 5 N NaOH (aqueous, 1 mL). Stir at 50° C. for about 30 min. Cool to RT and add excess AcOH (500 μL). Stir for about 45 min. Add this mixture to water (15 mL), whereupon a grey precipitate forms. Collect the solids by filtration and dissolve the solids in DMSO (4.5 mL). Purification and lyophilization of the volatiles affords 1190.

### Step 2:

Add 0.5 M/THF cyclopropyl magnesium bromide (300 μL, 0.15 mmol) to a solution of 1f3 (50 mg, 0.12 mmol) in THF (2 mL) at 0° C., and stir for about 1 h. Add water (100 μL) and evaporate the volatiles. Add DMSO (1.5 mL) and 5 N NaOH (aqueous, 0.3 mL) and stir at RT for about 1-2 h. Acidify with excess AcOH. Purification and lyophilization of the volatiles affords 1188.

### Example 7A

### Compound 1189

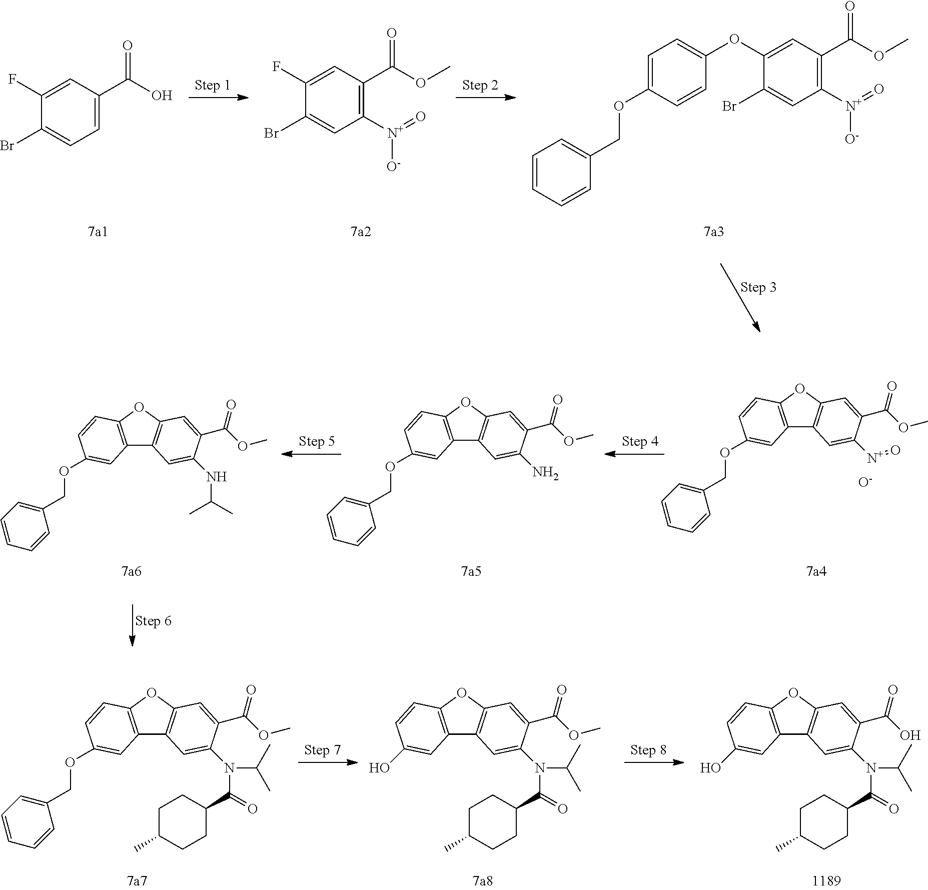

### Step 1:

Suspend 7a1 (19.9 g, 90.9 mmol) in concentrated sulfuric acid (150 mL) at RT and add KNO₃ (9.65 g, 95.4 mmol) portionwise. Stir the mixture for about 18 h, then pour slowly over 1.8 kg of ice. Stir until the ice melts and then filter the solids. Wash with water and dry at RT and ambient humidity. Dissolve the resulting solid in t-BME, and add freshly prepared diazomethane/t-BME solution until the intermediate acid is no longer detectable by RP-HPLC. Add AcOH to quench excess diazomethane, then wash with water and saturated NaHCO₃ (aqueous). Dry the organic portion over Na₂SO₄, filter and concentrate to give 7a2.

### Step 2:

Using the protocol described in Example 1C, Step 1, stir 7a2 (1.00 g, 3.60 mmol) and hydroquinone monobenzyl ether (756 mg, 3.78 mmol) in the presence of cesium carbonate (1.46 g, 4.50 mmol) to give 7a3.

### Step 3:

Using the protocol described in 1C, Step 2, combine 7a3 (1.41 g, 3.07 mmol) with potassium carbonate (1.27 g, 9.21 mmol), tricyclohexylphosphine tetrafluoroborate (236 mg, 0.62 mmol), DMA (10 mL), and palladium(II)acetate (147 mg, 0.22 mmol) at 130° C. for about 30 min to give 7a4.

### Step 4:

Stir a mixture consisting of 7a4, (674 mg, 2.32 mmol), stannous chloride (2.19 g, 11.6 mmol) and MeOH (50 mL) at 70° C. for about 6 h. Dilute the reaction with EtOAc (400 mL) and then add saturated NaHCO₃ (aqueous, 400 mL). Stir the biphasic mixture for about 2 days. Filter the solids and then collect the filtrate. Separate the layers and dry the organic portion over Na₂SO₄, filter and concentrate. The solid is trituated with a 25% t-BME/hexanes mixture, filtered and washed with hexanes to give 7a5.

### Step 5:

Add 4 M HCl/dioxane (363 μL, 1.45 mmol), 2-methoxy propene (1.11 mL, 11.6 mmol) and NaHB(OAc)₃ (770 g, 3.64 mmol) to a mixture of 7a5 (505 mg, 1.45 mmol) in DCM (30 mL). Stir at RT for about 2 h. Add saturated NaHCO₃ (aqueous) and stir for about 30 min. Dilute with t-BME and separate the layers. Wash the organic portion with brine, dry over MgSO₄, filter and concentrate to give 7a6.

### Step 6:

Heat a mixture consisting of 7a6 (104 mg, 0.27 mmol), 1a8 (51 mg, 0.30 mmol), pyridine (108 μL, 1.34 mmol) and DCE (2 mL) at 150° C., for 20 min in a microwave. Dilute with EtOAc. Wash with 1 M HCl (aqueous) and brine, then dry over MgSO₄, filter and concentrate. Purification of the residue by flash chromatography (EtOAc/Hexanes) affords 7a7.

### Step 7:

Using the protocol described in Example 1N, Step 1, 7a7 (80 mg, 0.16 mmol) is converted to 7a8.

### Step 8:

Using the protocol described in Example 1A, Step 7, 7a8 (20 mg, 0.047 mmol) is converted to 1189.

### Example 7B

### Compound 2025

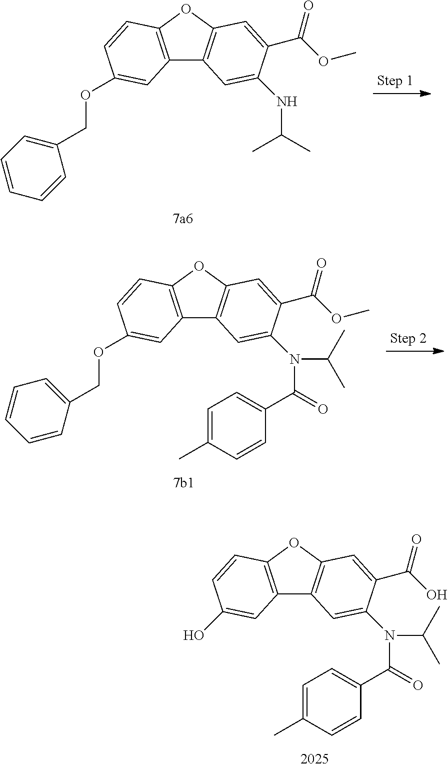

### Step 1:

Using the protocol described in Example 7A, Step 6, 7a6 (102 mg, 0.26 mm converted to 7b1.

### Step 2:

Expose a mixture of 7b1 (60 mg, 0.12 mmol), MeOH (30 mL), TFA and Pearlman's catalyst with stirring to a hydrogen atmosphere (1 atm) for about 2 h. Filter the catalyst and evaporate the volatiles. Add DMSO (1.5 mL) and 5 N NaOH (aqueous, 0.3 mL) and stir at RT for about 30 min. Acidify with excess AcOH. Purification and lyophilization of the volatiles affords 2025.

### Example 8A

### Compounds 2003, 2005, 2006 and 2022

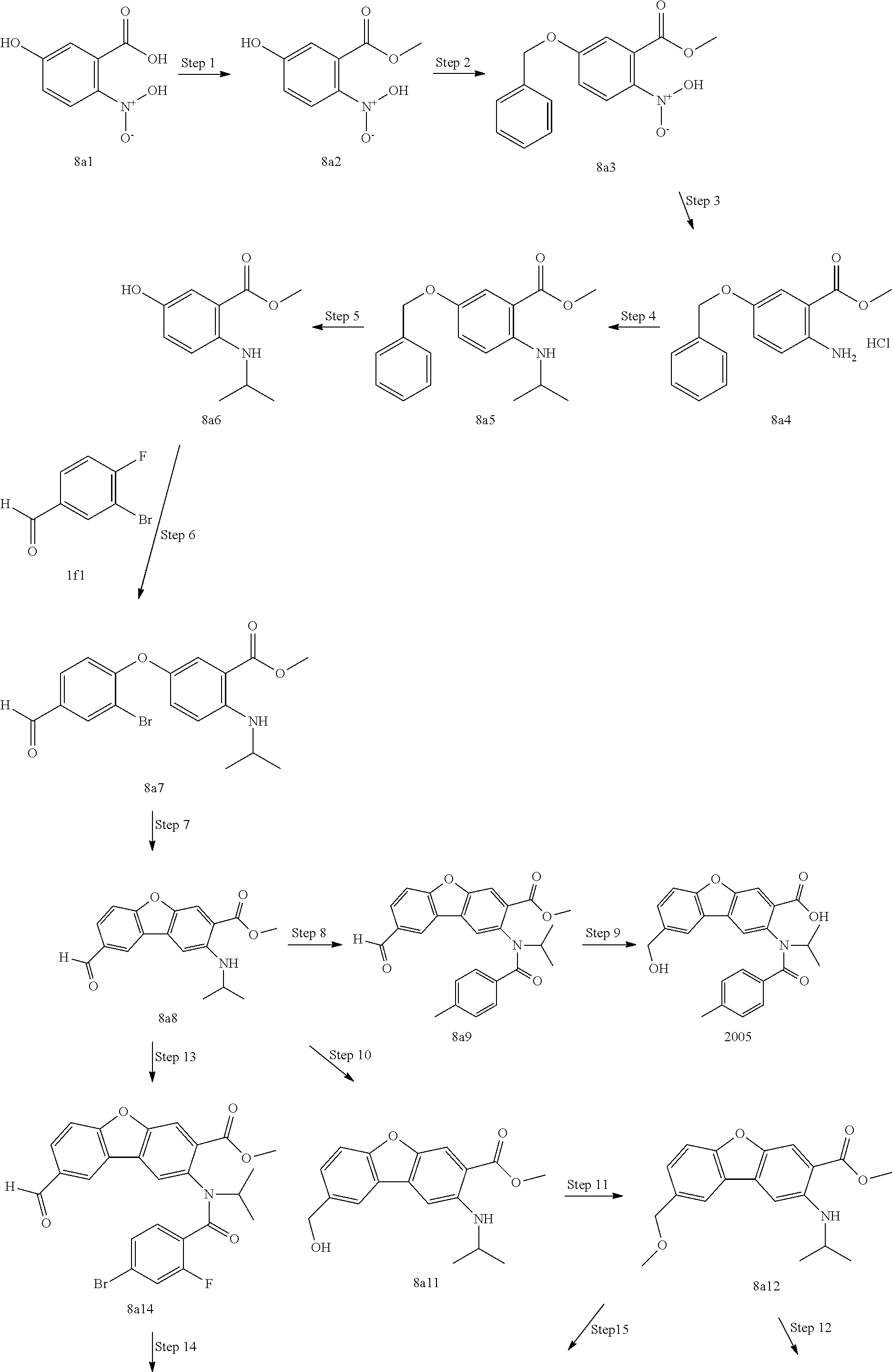

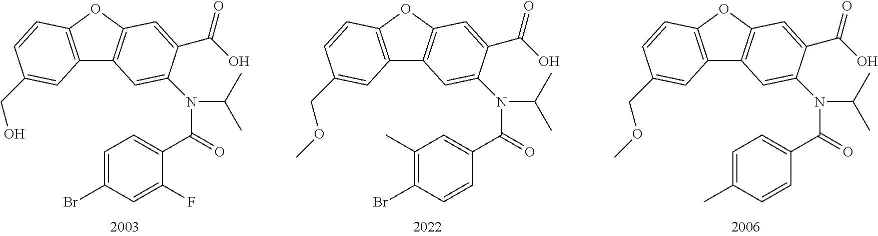

### Step 1:

Add thionyl chloride (40 mL, 0.55 mol) dropwise to 5-hydroxy-2-nitrobenzoic acid 8a1 (50.0 g, 0.270 mol) in MeOH (500 mL). Heat to 76° C. for about 2 h. Add a further portion of thionyl chloride (20 mL, 0.27 mol) dropwise and continue heating for about 18 h. Add a final portion of thionyl chloride (20 mL, 0.27 mol) and continue heating for about 1 h. Mow the mixture to cool to RT and concentrate under reduced pressure. Dilute with EtOAc. Wash with saturated NaHCO₃ (aqueous) and brine, dry over MgSO₄, filter and concentrate to dryness. Crystallize the residue with DCM and hexanes to provide 8a2.

### Step 2:

Stir a mixture consisting of 8a2 (51.2 g, 0.26 mol), potassium carbonate (150 g, 1.09 mmol), benzyl bromide (39 mL, 0.33 mol) and acetone at RT for about 18 h. Filter the solids and collect and concentrate the filtrate. Dilute the filtrate with EtOAc. Wash with water, then brine, dry over MgSO₄, filter and evaporate to dryness. Crystallize the residue with EtOAc and hexanes to provide 8a3.

### Step 3:

Using the protocol described in Example 1A, Step 2, treat 8a3 (68.4 g, 0.24 mol with elemental iron (225 g, 4.0 mol), acetic acid (95 mL) and EtOH (1.2 L). Heat to reflux to give a solid following workup and crystallization from DCM and hexanes. Dissolve this solid in Et₂O (400 mL) and then add 2 M HCl/Et₂O (180 mL) and stir for about 2 h. Filter the solids and dry to provide 8a4.

### Step 4:

Using the protocol described in Example 1A, Step 3, convert 8a4 (105.2 g, 0.36 mol) to give 8a5 following crystallization from EtOAc and hexanes.

### Step 5:

Hydrogenate a mixture consisting of 8a5 (9.3 g, 31.1 mmol), EtOAc (200 mL), MeOH (200 mL), and 10% Pd/C (0.9 g) at 30 psi H₂ (g) for about 6-8 h at RT. Filter the solids and concentrate to provide an oil. Trituate with hexanes to give 8a6.

### Step 6:

Using the protocol described in Example 1C, Step 1, 8a6 (4.80 g, 22.9 mmol) is converted to 8a7,

### Step 7:

Using the protocol described in Example 1C, Step 2, 8a7 (1.50 g, 3.82 mmol) is converted to 8a8.

### Step 8:

Heat a mixture of 8a8 (60 mg, 0.19 mmol), p-toluoyl chloride (51 μL, 0.38 mmol) and pyridine (1 mL) to 70° C. for about 5 h. Add a further portion of p-toluoyl chloride (51 μL, 0.3 8 mmol) and continue heating for about a further 3 h. Evaporate the volatiles and dilute with EtOAc. Wash with 10% citric acid (aqueous) and saturated NaHCO₃ (aqueous). Pass the organic portion through a pad of EXTRELUT®, concentrate, then purify by flash chromatography (EtOAc/Hexanes) to provide 8a9.

### Step 9:

Using the protocol described in Example 1F, Step 3, 8a9 (40 mg, 0.093 mmol) is converted to 2005.

### Step 10:

Using the protocol described in Example 4A, Step 1, 8a8 (1.79 g, 5.75 mmol) is converted to 8a11.

### Step 11:

Add a solution of 8a11 (600 mg, 1.92 mmol) in DMF (9 mL) dropwise to a mixture of 60% w/w NaH/mineral oil (92 mg, 2.30 mmol). DMF (9 mL) and iodomethane (180 μL, 2.88 mmol) at −10° C. Stir for about 2 h, then quench with saturated NH₄Cl (aqueous). Dilute with EtOAc and water. Separate and wash the organic portion with to brine, dry over MgSO₄, filter and concentrate. Purification of the residue by flash chromatography (EtOAc/Hexanes) affords crude 8a12.

### Step 12:

Heat a mixture of crude 8a12 (45 mg, 0.14 mmol), p-toluoyl chloride (36 μL, 0.27 mmol) and pyridine (1.5 mL) to 70° C. for about 5 h. Evaporate the volatiles and add DMSO (1 mL) and 5 N NaOH (aqueous, 0.3 mL). Stir at RT for about 3 h. Acidify with excess AcOH. Purification and lyophilization of the volatiles affords 2006.

### Step 13:

Suspend 4-bromo-2-fluorobenzoic acid (55 mg, 0.25 mmol) in thionyl chloride (500 μL) and add DMF (10 μL). Stir for about 18 h at RT. Evaporate the volatiles, then co-evaporate with toluene. Dissolve the residue in pyridine (1 mL) and add solid 8a8 (60 mg, 0.19 mmol). Stir at RT for about 3 h. Evaporate the volatiles and dilute with EtOAc. Wash with 10% citric acid (aqueous) and saturated NaHCO₃ (aqueous). Pass the organic portion through a pad of EXTRELUT®, concentrate, and purify the residue with flash chromatography (EtOAc/Hexanes) to provide 8a14.

### Step 14;

Using the protocol described in Example 1F, Step 3, 8a14 (45 mg, 0.088 mmol) is converted to 2003.

### Step 15:

Suspend 4-bromo-3-methylbenzoic acid (39 mg, 0.18 mmol) in thionyl chloride (600 μL) and add DMF (10 μL). Stir for about 1 h at 70° C. Evaporate the volatiles, then co-evaporate with DCE. To the remaining residue, add 8a12 (20 mg, 0.06 mmol) in DCE and add pyridine (25 μL, 0.31 mmol). Heat to 150° C. for 15 min in a microwave. Dilute with THF and treat with polystyrene-trisamine for about 2 h at RT. Filter the solids, collect the filtrate and concentrate. Add DMSO (1 mL) and 5 N NaOH (aqueous, 0.15 mL) and stir at RT for about 3 h. Acidify with excess AcOH. Purification and lyophilization of the volatiles affords 2022.

### Example 9A

### Compound 2018

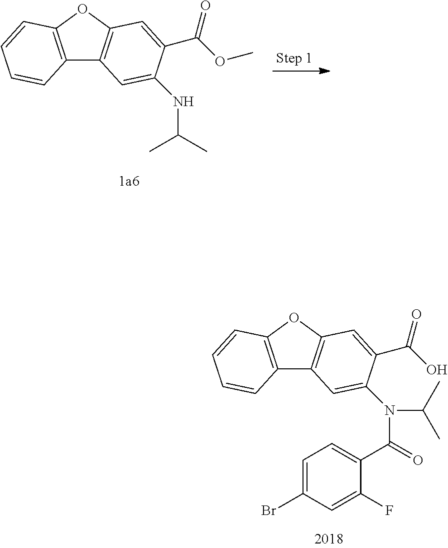

### Step 1:

Using the protocol described in Example 8A, Step 15, 1a6 (20 mg, 0.071 mmol) is converted to 2018.

### Example 10A

### Compounds 2009, 2011 and 2012

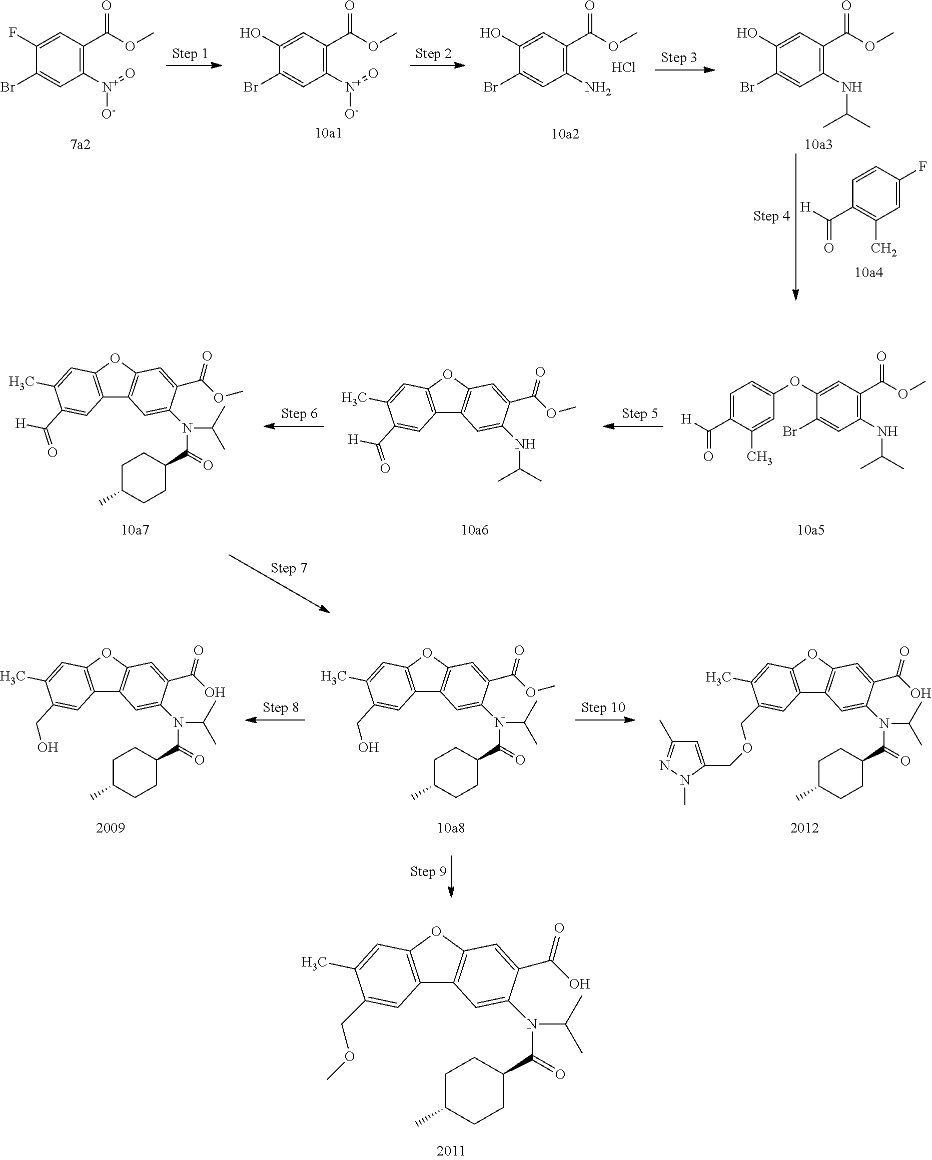

### Step 1:

Using the procedure outlined in Tetrahedron Letters, 2002, 43, pp 3585-3587, add 60% w/w NaH/mineral oil (1.80 g, 45 mmol) to 7a2 (5.00 g, 18.0 mmol), 2-(methylsulfonyl)ethanol (3.35 g, 27 mmol) and DMF (35 mL) in four equal portions over a period of about 20 min. After stirring at RT for about 1 h, add a further portion of 60% w/w NaH/mineral oil (300 mg) and stir for about 30 min. Quench with AcOH, dilute with water and extract with t-BME. Dry the organic portion over Na₂SO₄ filter and concentrate. Purification of the residue by flash chromatography (EtOAc/Hexanes) affords 10a1

### Step 2:

Combine 10a1 (4.27 g, 15.5 mmol) with MeOH (100 mL) and stannous chloride (11.7 g, 61.9 mmol) to give a solid following workup. Dissolve this solid in t-BME (100 mL), add 2 M HCl/Et₂O (20 mL) and stir for about 1 h. Evaporate the volatiles and trituate the residue with t-BME and hexanes to give 10a2.

### Step 3:

Using the protocol described in Example 1A, Step 3, 10a2 (3.96 g, 0.14 mmol) is converted to 10a3 following purification y flash chromatography (EtOAc/Hexanes)

### Step 4:

Using the protocol described in Example 1C, Step 1, combine 2-methyl-4-fluoro benzaldehyde 10a4 (365 mg, 2.64 mmol) and 10a3 (610 mg, 2.12 mmol) in the presence of cesium carbonate (1.20 g, 7.70 mmol) at 80° C. to give 10a5.

### Step 5:

Using the protocol described in Example 1C, Step 2, 10a5 (470 mg, 1.16 mmol) is converted to 10a6.

### Step 6:

Using the protocol described in Example 7A, Step 6, 10a6 (200 mg, 0.62 mmol) is converted to 10a7.

### Step 7:

Using the protocol described in Example 4A, Step 1, 10a7 (173 mg, 0.38 mmol) is converted to 10a8.

### Step 8:

Using the protocol described in Example 1A, Step 7, 10a8 (20 mg, 0.044 mmol) is converted to 2009.

### Step 9:

Using the protocol described in Example 1H, Step 5, 10a8 (30 mg, 0.066 mmol) is converted to 2011.

### Step 10:

Using the protocol described in Example 1H, Step 5, convert 10a8 (50 mg, 0.11 mmol) in the presence of 5-(chloromethyl)-1,3-dimethyl-1H-pyrazole (24 mg, 0.17 mmol) to give 2012.

### Example 11A

### Compounds 2014, 2015, 2020 and 2021

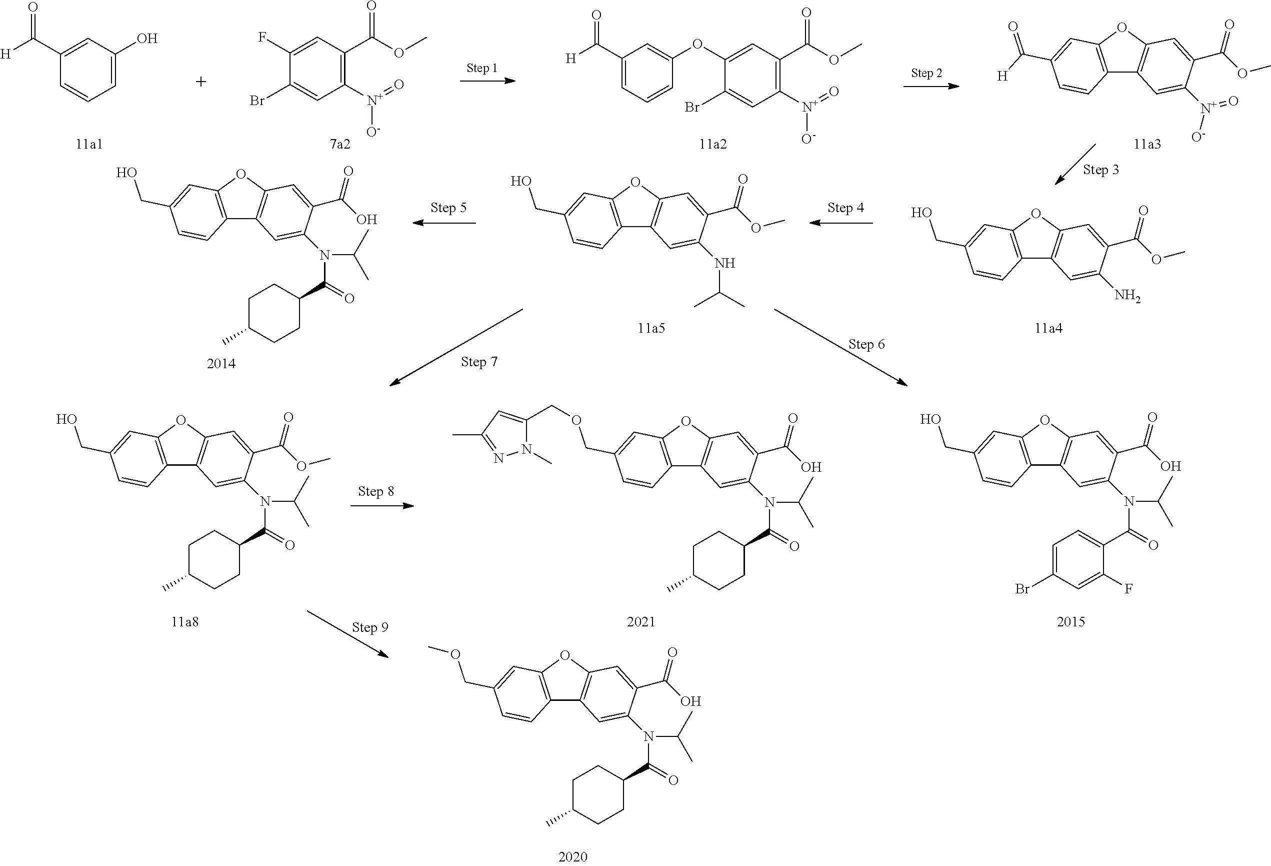

### Step 1:

Using the protocol described in Example 1C, Step 1, stir 7a2 (2.00 g, 7.21 mmol) and 3-hydroxybenzaldehyde 11a1 (972 mg, 7.60 mmol) in the presence of cesium carbonate (2.84 g, 8.63 mmol) to give 11a2.

### Step 2:

Using the protocol described in Example 1C, Step 2, combine 11a2 (2.33 g, 6.10 mmol) with potassium carbonate (2.52 g, 18.3 mmol), tricyclohexylphosphine tetrafluoroborate (468 mg, 1.23 mmol), DMA (35 mL) and palladium(II)acetate (292 mg, 1.27 mmol) at 130° C. for about 30 min to give 11a3.

### Step 3:

Using the protocol described in Example 1N, Step 1, 11a3 (840 mg, 2.81 mmol) is converted to 11a4 following purification by flash chromatography (EtOAc/Hexanes).

### Step 4:

Using the protocol described in Example 7A, Step 5, 11a4 (340 mg, 1.23 mmol) is converted to 11a 5,

### Step 5:

Heat a mixture consisting of 11a5 (25 mg, 0.080 mmol), 1a8 (32 mg, 0.20 mmol), pyridine (65 μL; 0.80 mmol) and DCE (0.5 mL) and heat at 150° C. for 20 min in a microwave. Evaporate the volatiles, add DMSO (1.5 mL) and 5 N NaOH (aqueous, 0.3 mL) and stir at RT for about 2 h. Acidify with excess AcOH. Purification and lyophilization of the volatiles affords 2014.

### Step 6:

Suspend 4-bromo-2-fluorobenzoic acid (44 mg, 0.20 mmol) in thionyl chloride (300 μL) and add DMF (10 ƒL), then stir for about 2 h at RT. Evaporate the volatiles, then co-evaporate with toluene. Dissolve the residue in DCE (0.5 mL), add pyridine (65 μL, 0.80 mmol), then 11a5 (25 mg, 0.080 mmol) and heat at 150° C. for 20 min in a microwave. Evaporate the volatiles, add DMSO (1.5 mL) and 5 N NaOH (aqueous, 0.30 mL) and stir at RT for about 2 h. Acidify with excess AcOH. Purification and lyophilization of the volatiles affords 2015.

### Step 7;

Heat a mixture consisting of 11a5 (126 mg, 0.40 mmol), 1a8 (161 mg, 1.01 mmol), pyridine (325 μL, 4.21 mmol) and DCE (6 mL) and heat at 150° C. for 20 min in a microwave. Evaporate the volatiles, add DMSO (3 mL) and 5 N NaOH (aqueous, 0.5 mL) and stir at RT for about 1 h. Pour the mixture into 0.5 M KHSO₄ (aqueous, 25 mL) and extract with EtOAc. Wash the organic portion with water and brine, dry over Na₂SO₄, filter and evaporate. Dissolve the residue in t-BME, then add freshly prepared diazomethane/ether solution. Titrate until characteristic yellow persists. Evaporate and purify the residue with flash chromatography (EtOAc/Hexanes) to give 11a8.

### Step 8:

Using the protocol described in Example 10A, Step 10, 11a8 (40 mg, 0.091 mmol) is converted to 2021.

### Step 9:

Using the protocol described in Example 1H, Step 5, 11a8 (30 mg, 0.069 mmol) is converted to 2020.

### Example 12A

### Compounds 2027 and 2029

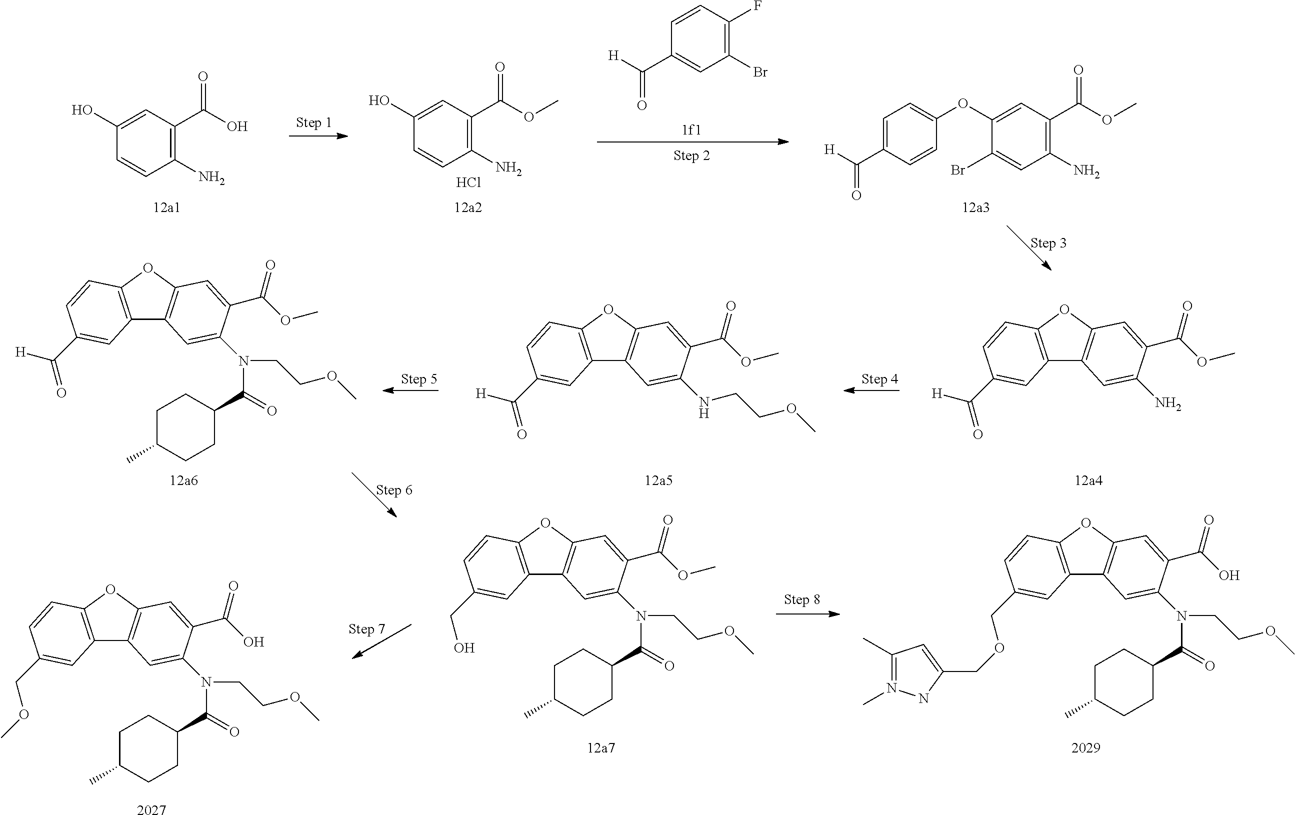

### Step 1:

Add thienyl chloride (1.9 mL, 26.1 mmol) to a solution of 2-amino-5-hydroxybenzoic acid, 12a1 (2.00 g, 13.1 mmol) in MeOH (50 mL). Stir at 70° C. for about 48 h. Add a further portion of thionyl chloride (1.9 mL, 26.1 mmol) and continue heating for about 72 h. Concentrate to dryness, then suspend solids in t-BME. Stir for about 24 h, then filter and air dry to provide 12a2.

### Step 2:

Using the protocol described in Example 1C, Step 1, combine 3-bromo-4-fluoro benzaldehyde 1f1 (2.53 g, 12.5 mmol) and 12a2 (2.31 g, 11.3 mmol) in the presence of cesium carbonate (7.37 g, 22.7 mmol) to give 12a3.

### Step 3:

Using the protocol described in Example 11A, Step 2, 12a3 (1.77 g, 5.06 mmol) is converted to 12a4.

### Step 4:

Stir a mixture consisting oil 12a4 (200 mg, 0.74 mmol), 2-bromoethyl methyl ether (700 μL, 7.43 mmol), KI (616 mg, 3.71 mmol), DIPEA (1300 μL, 7.43 mmol) and DMF (5 mL) at 120° C. for about 18 h. Dilute with EtOAc, wash with 1 M NaOH (aqueous), water and brine, dry over Na₂SO₄, filter and evaporate. Purification of the residue by flash chromatography (EtOAc/Hexanes) affords 12a5.

### Step 5:

Using the protocol described in Example 7A, Step 6, 12a5 (75 mg, 0.23 mmol) is converted to 12a6.

### Step 6:

Using the protocol described in Example 4A, Step 1, 12a6 (75 mg, 0.17 mmol) is converted to 12a7.

### Step 7:

Using the protocol described in Example 1H, Step 5, 12a7 (25 mg, 0.055 mmol) is converted to 2027.

### Step 8:

Using the protocol described in Example 10A, Step 10, 12a7 (47 mg, 0.10 mmol) to give 2029.

### Example 13A

### Compounds 2028 and 2030

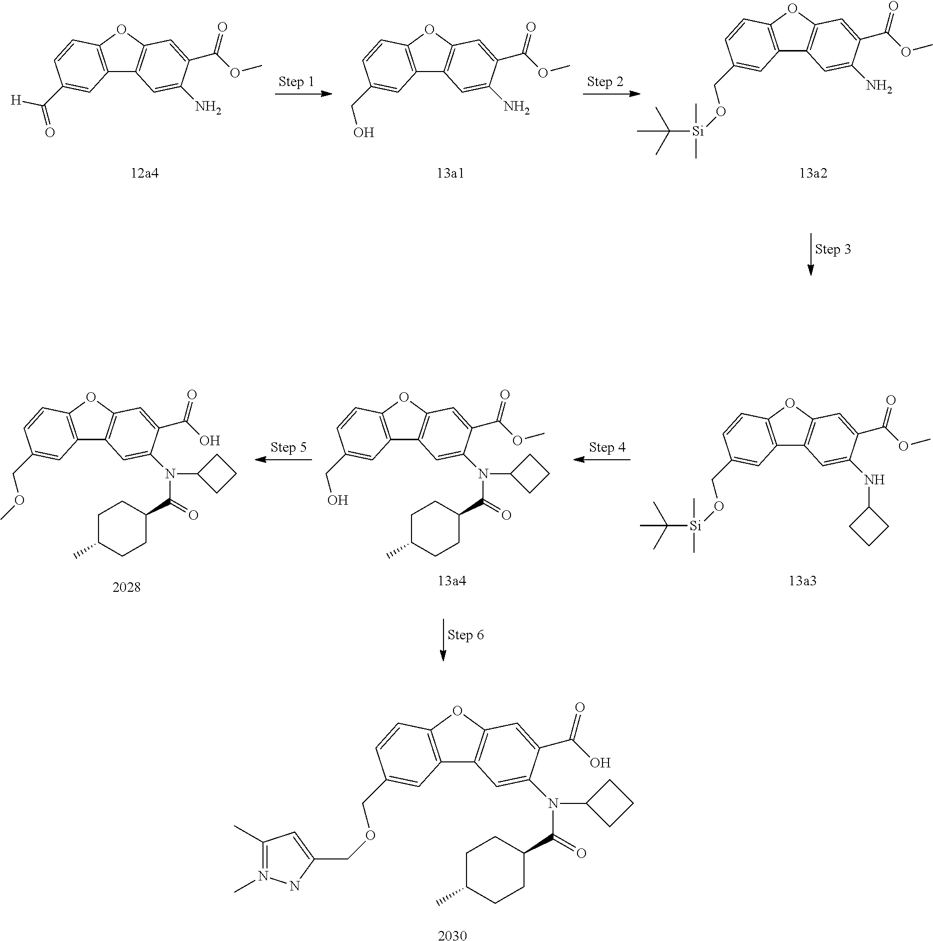

### Step 1:

In a fashion analogous to that for the production of 4a1 (Example 4A, Step 1), convert 12a4 (580 mg, 2.15 mmol) to give 13a1.

### Step 2:

Stir a mixture of 13a1 (570 mg, 2.10 mmol), DMF (5 mL), imidazole (429 mg, 6.30 mmol) and t-butyldimethylsilyl chloride (633 mg, 4.20 mmol) at RT for about 1 h. Dilute with t-BME, wash successively with portions of 10% citric acid (aqueous), saturated NaHCO₃ (aqueous), water and brine. Dry over Na₂SO₄, filter and evaporate. Purification of the residue by flash chromatography (EtOAc/Hexanes) affords 13a2.

Step 3: Following a Protocol Similar to that Described at Pages 60-61 of WO 06/119646.

Stir a mixture consisting of 13a2 (200 mg, 0.52 mmol), cyclobutanone (77 μL, 1.04 mmol), dibutyltin chloride (8 mg, 0.026 mmol), phenylsilane (70 μL, 0.57 mmol) and THF (5 mL) for about 24 h at 70° C. Add further portions of cyclobutanone (77 μL, 1.04 mmol), dibutyltin chloride (8 mg, 0.026 mmol) and phenylsilane (70 μL, 0.57 mmol), and continue heating for about 24 h. Dilute with t-BME, and wash with saturated NaHCO₃ (aqueous). Dry over Na₂SO₄, filter and concentrate. Purification of the residue by flash chromatography (EtOAc/Hexanes) affords 13a3.

### Step 4:

Heat a mixture consisting of 13a3 (165 mg, 0.37 mmol), 1a8 (120 mg, 0.75 mmol), pyridine (152 μL, 1.68 mmol) and DCE (2.5 mL) at 150° C. for 20 min in a microwave. Dilute with t-BME and 1 M HCl (aqueous) and separate the layers. Wash the organic portion with brine, dry over MgSO₄, filter and concentrate. Dissolve the residue in THF (5 mL) and add 1 M TBAF/THF solution (1 mL). Stir for about 1 h. Evaporate the volatiles and add MeOH (5 mL), OMSO (2 mL) and 5 N NaOH (aqueous, 1 mL). Stir at 50° C. for about 3 h and then at RT for about 18 h. Dilute with t-BME and 1 M HCl (aqueous) and separate the layers. Wash the organic portion with brine, dry over MgSO₄, filter and concentrate. Dissolve the residue in t-BME and add excess freshly prepared diazomethaneiether solution. Concentrate and purify the residue with flash chromatography (EIOAc/hexanes) to give 13a4.

### Step 5:

Using the protocol described in Example 1H, Step 5, 13a4 (25 mg, 0.056 mmol) is converted to 2028.

### Step 6:

Using the protocol described in Example 10A, Step 10, 13a4 (50 mg, 0.11 mmol) is converted to 2030.

### Example 14A

### Compound 1017

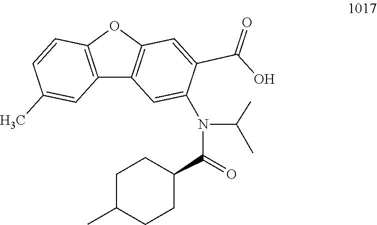

Using the protocol described in Example 1A, Steps 1-7, synthesize 1017 beginning with 4-methyl-2-bromophenol.

### Example 15A

### Compound 3002

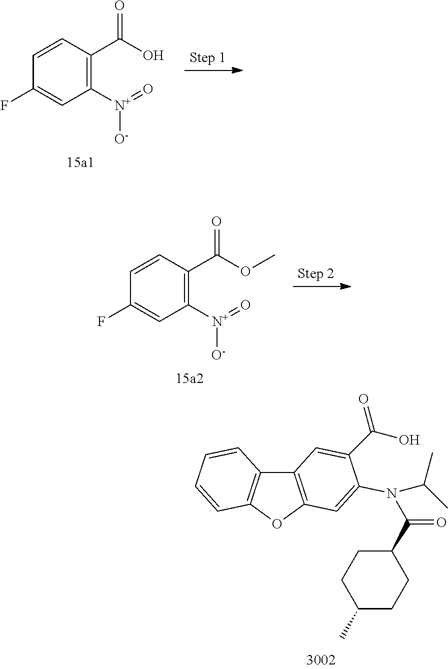

### Step 1:

Using the protocol described in Example 12A, Step 1, 15a1 (25 g, 135 mmol) is converted to 15a2.

### Step 2:

Using the protocol described in Example 1A, Steps 1-7, synthesize 3002 beginning with 2-bromophenol and 15a2.

### Example 16A

### Compound 3001

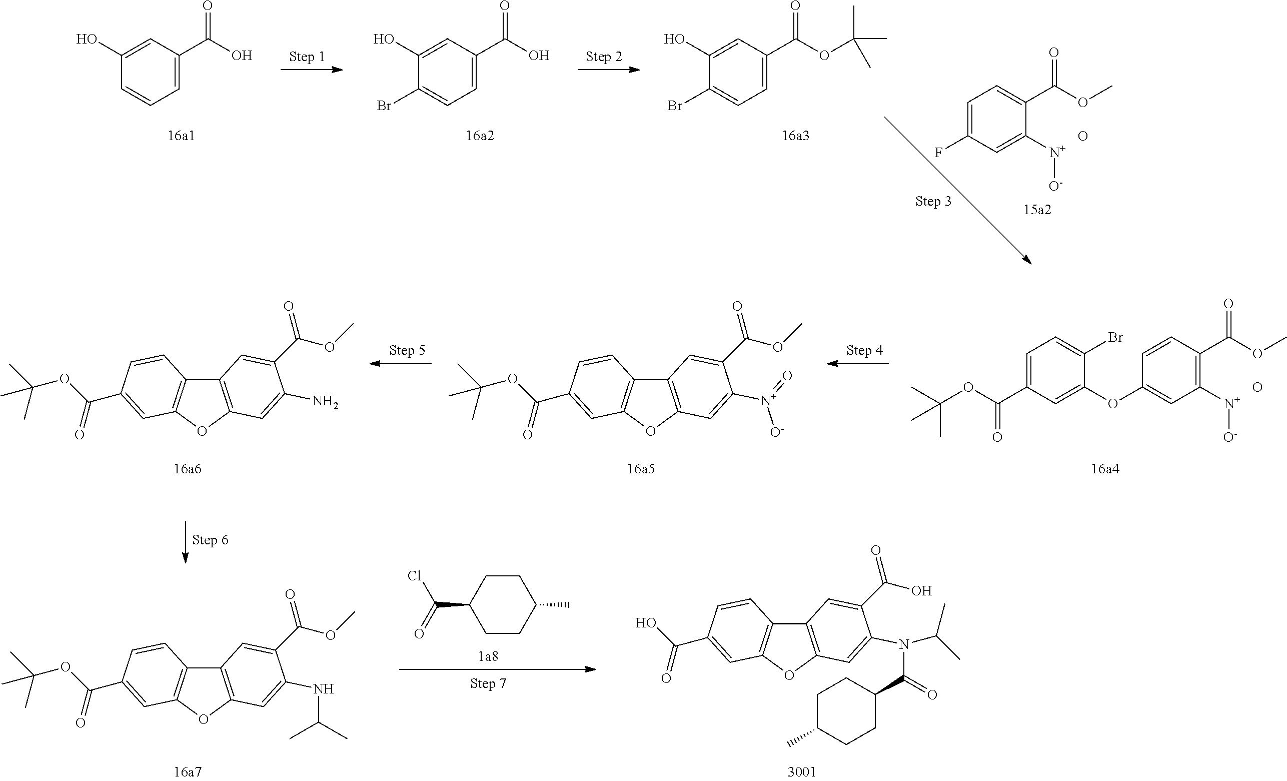

### Step 1:

Reference: L. W. Lawrence Woo et al, J. Med. Chem. 2007, 50, 3540-3560.

Dissolve 3-hydroxybenzoic acid 16a1 (18.20 g, 130.4 mmol) in acetic acid (180 mL) and cool to 0° C. Add bromine (21.34 g, 133.2 mmol) as a solution in acetic acid dropwise over about 30 min. Evaporate the solvent under reduced pressure to give a wet solid mass. Add water (200 mL) and stir initially at RT, then warm slowly to 80° C., stirring constantly. Cool slowly the now homogeneous mixture to 0° C., whereupon a solid forms. Add a further portion of water (200 mL) and stir the suspension at RT overnight. Filter and dry the solids to provide 16a2.

### Step 2:

Reference: Ulrich Widmer, Synthesis, 1983, 135-136.

Dissolve 16a2 (4.55 g, 20.97 mmol) in anhydrous toluene (20 mL) and heat to 80° C. Add dimethylformamide di-t-butylacetal (10 mL, 42.7 mmol) portionwise over about a 2 h period. Cool the mixture to RT. Evaporate the volatiles and purify the residue with flash chromatography (EtOAc/hexanes) to give 16a3.

### Step 3:

In a fashion analogous to that for the production of 1c3 (Example 1C, Step 1), combine 15a2 (565 mg, 2.95 mmol) and 16a3 (729 mg, 2.67 mmol) in the presence of cesium carbonate (996 mg, 3.03 mmol) to give 18a4.

### Step 4:

In a fashion analogous to that for the production of 1c4 (Example 1C, Step 2), combine 16a4 (779 mg, 1.72 mmol) with potassium carbonate (724 mg, 5.24 mmol), tricyclohexylphosphine tetrafluoroborate (128 mg, 0.35 mmol), DMA (5 mL) and palladium(II)acetate (80 mg, 0.35 mmol) at 130° C. for about 24 h. Dilute the reaction with EtOAc, wash with 0.5 M KHSO₄ (aqueous) and brine, dry over MgSO₄, filter and evaporate. Purify the residue with flash chromatography (EtOAc/hexanes) to give 16a5.

### Step 5:

In a fashion analogous to that for the production of 1n1 (Example 1N, Step 1), convert 16a5 (250 mg, 0.67 mmol) to give 16a6.

### Step 6:

Using the protocol described in Example 7A, Step 5, 16a6 (240 mg, 0.70 mmol) is converted to 16a7.

### Step 7:

Heat a mixture consisting of 16a7 (25 mg, 0.065 mmol), 1a8 (21 mg, 0.13 mmol), pyridine (42 μL, 0.52 mmol) and DCE (700 mL) at 150° C. for 20 min in a microwave. Evaporate the volatiles. Add TFA (1 mL) and stir for about 1 h at RT, then evaporate. Add DMSO (1.5 mL) and 5 N NaOH (aqueous, 0.30 mL) and stir at RT for about 2 h. Acidify with excess AcOH. Purification and lyophilization of the volatiles affords 3001.

### Example 17A

### Compounds 3003 and 3004

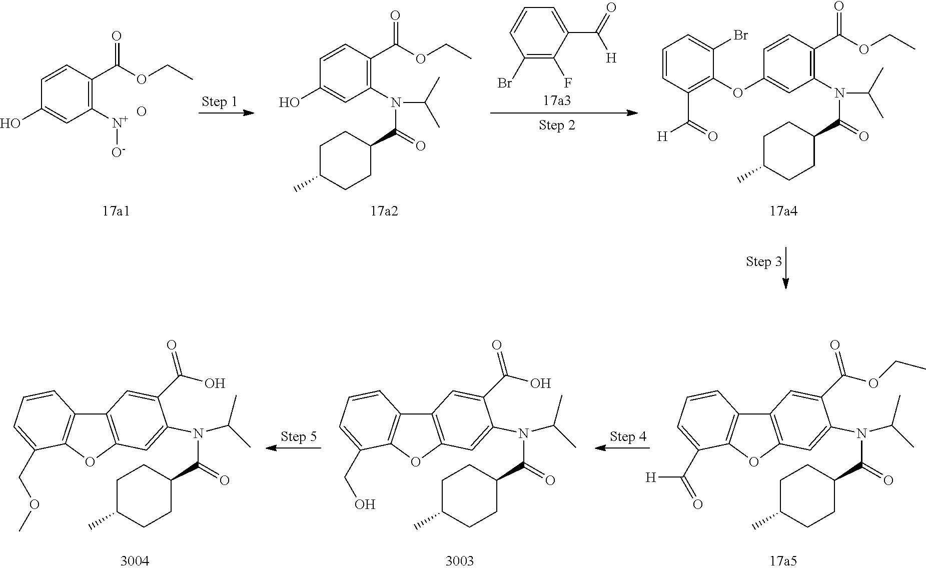

### Step 1:

Using the protocol described for the production of 1c2, 17a1 is converted to 17a2.

### Step 2:

Using the protocol described in Example 1C, Step 1, stir 17a2 (270 g, 0.78 mmol) and 2-fluoro-3-bromobenzaldehyde 17a3 (245 mg, 1.21 mmol) in the presence of cesium carbonate (380 mg, 117 mmol) to give 17a4.

### Step 3:

Using the protocol described in Example 11A, Step 2, 17a4 (100 g, 0.19 mmol) is converted to 17a5.

### Step 4:

Using the protocol described in Example 1F. Step 3, 17a5 (65 mg, 0.14 mmol) is converted to 3003.

### Step 5:

Add to a mixture of 3003 (25 mg, 0.059 mmol) in THF (1 mL), a freshly prepared solution of diazomethane/t-BME solution. Evaporate the THF and add DMF (2 mL), 60% w/w NaH/mineral oil (10 mg) and iodomethane (20 μL). Stir for about 1 h at RT. Add DMSO (1 mL) and 5 N NaOH (aqueous, 0.5 mL) and stir at RT for about 1 h. Acidify with excess TFA. Purification and lyophilization of the volatiles affords 3004.

### Example 18A

### Compound 2033

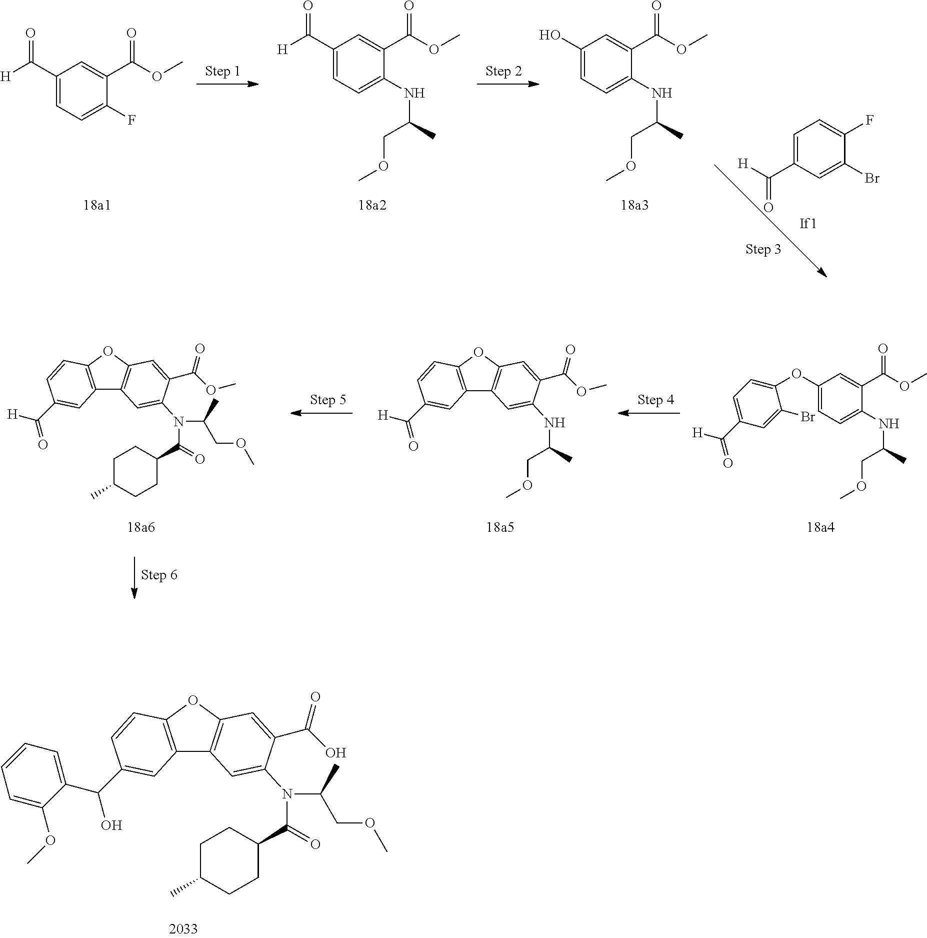

### Step 1:

Potassium carbonate (400 mg, 2.89 mmol) is added to a DMSO (4.0 mL) solution of fluoride 18a1 (438 mg, 2.4 mmol) and (S)-(+)-1-methoxy-2-propylamine (858 mg, 9.63 mmol). The mixture is heated at 70° C. for about 20 h, cooled to RT and diluted with water. Concentrated HCl is then added to make the mixture acidic. The solution is stirred at RT for about 1 h, basified with aqueous 2.5 N NaOH and extracted with EtOAc. The organic phase is washed with brine, dried over Na₂SO₄, filtered and concentrated under reduced pressure. The crude product 18a2 is used directly in the next step.

### Step 2:

Hydrogen peroxide (374 μL, 3.3 mmol) is added to a 0° C. MeOH (10 mL) solution of the aldehyde 18a2 and sulfuric acid (180 μL, 2.9 mmol). The solution is stirred at 0° C. for about 2 h, basified with aqueous 2.5 N NaOH and extracted with EtOAc. The organic phase is washed with brine, dried over Na₂SO₄, filtered and concentrated under reduced pressure. The crude residue is purified by flash chromatography to afford phenol 18a3.

### Step 3:

Using the protocol described in Example 1C, Step 1, combine 18a3 (150 mg, 0.63 mmol) and 1f1 (140 mg, 0.69 mmol) in the presence of cesium carbonate (407 mg, 1.25 mmol) to give 18a4.

### Step 4:

Using the protocol described in Example 11A, Step 2, 18a4 (225 mg, 0.53 mmol) is converted to 18a5.

### Step 6:

Using the protocol described in Example 7A, Step 6, 18a5 (135 mg, 0.39 mmol) is converted to 18a6.

### Step 6:

Using the protocol described in Example 6A, Step 2, convert 18a6 (55 mg, 0.12 mmol) with 1 M 2-methoxyphenyl magnesium bromide/THF (150 μL, 0.15 mmol) to 2033.

### Example 19A

### Compound 2032

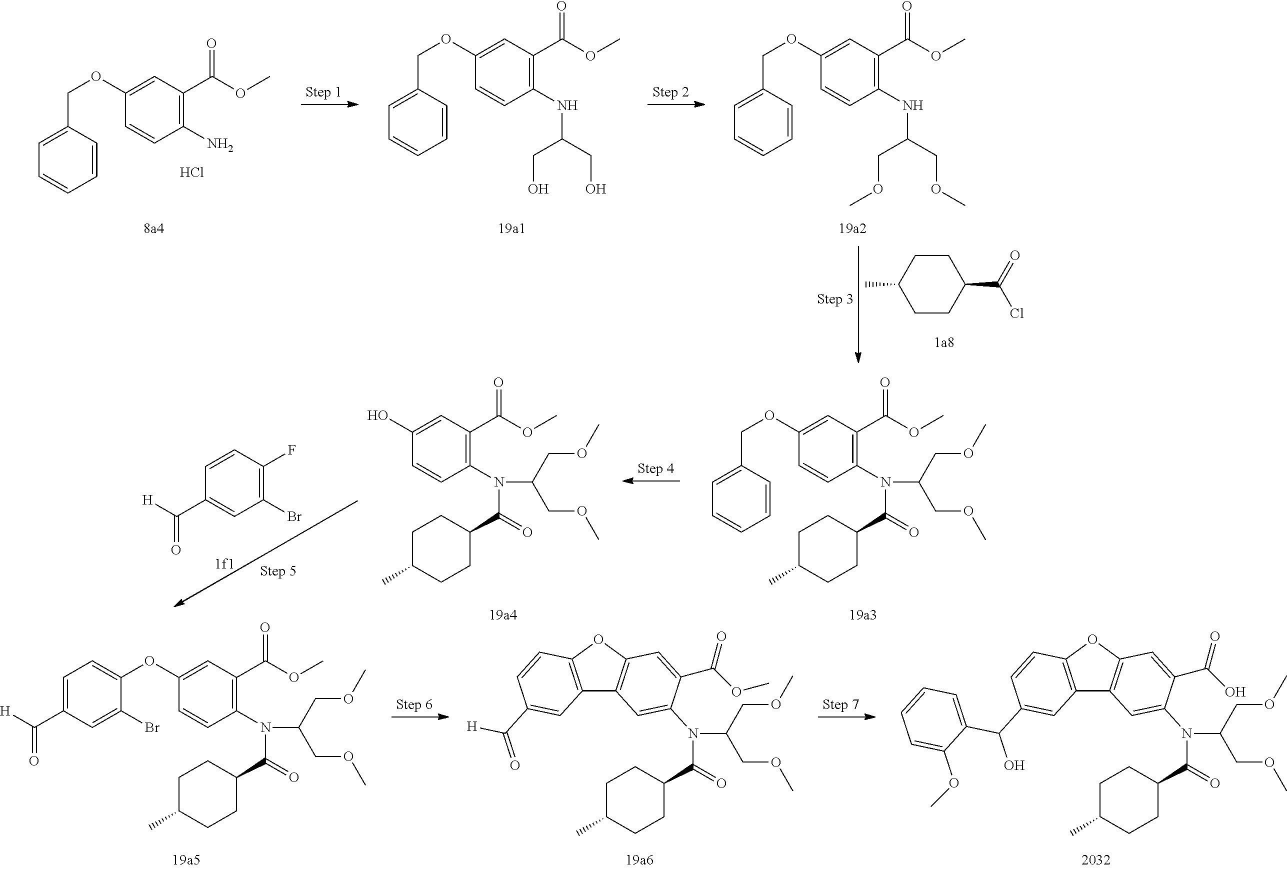

### Step 1:

Add to a mixture of 8a4 (60 g, 0.204 mol) in MeOH (2 L), 1,3-dihydroxyacetone (113 g, 1.25 mol) and stir at RT for about 15 min. Add NaHB(OAc)₃ (64.1 g, 1.02 mol) as a solution in MeOH (200 mL) and stir at RT for about 2-3 h. Add saturated NaHCO₃ (aqueous, 500 mL) and evaporate the MeOH. Extract with EtOAc, wash the organic portion with water and brine, dry over Na₂SO₄, filter and evaporate. Purify the residue with flash chromatography (EtOAc/hexanes) to give 19a1.

### Step 2:

Add to a mixture of 19a1 (50 g, 181 mmol), DMF (200 mL) and methyl iodide (77 g, 542 mmol), a suspension of NaH (7 g, 289 mmol) in DMF (200 mL) at RT. Stir overnight, then quench with saturated NH₄Cl (aqueous, 200 mL) and extract with EtOAc. Wash the organic portion with water and brine. Dry over Na₂SO₄, filter and concentrate. Purify the residue with flash chromatography (EtOAc/hexanes) to give 19a2.

### Step 3:

Reflux a mixture of 19a2 (10.0 g, 27.8 mmol), toluene (200 mL), pyridine (11.0 g, 139 mmol) and 1a8 (6.7 g, 41.7 mmol) for about 2 days. Cool to RT, filter the solids and discard. Collect the filtrate, concentrate and purify the residue with flash chromatography (EtOAc/hexanes) to give 19a3.

### Step 4:

Shake a mixture of 19a3 (12.63 g, 26.1 mmol), 10% Pd(OH)₂/C, EtOAc (75 mL) and MeOH (75 mL) under a 10 psi H₂ (g) atmosphere for about 18 h. Filter the mixture through Celite®, collect the filtrate and concentrate. Trituate the crude material with hexanes, filter and dry the solids. Dissolve the material in a 50% mixture of MeOH/EtOAc (200 mL), add activated charcoal (10 g) and reflux for about 1 h. Filter and evaporate to give 19a4.

### Step 5:

In a fashion analogous to that for the production of 1c3 (Example 1C, Step 1), combine 19a4 (150 mg, 0.38 mmol) and 1f1 (85 mg, 0.42 mmol) in the presence of cesium carbonate (248 mg, 0.76 mmol) to give 19a5.

### Step 6:

Using the protocol described in Example 11A, Step 2, 19a5 (180 mg, 0.31 mmol) is converted to 19a6.

### Step 7:

Using the protocol described in Example 6A, Step 2, convert 19a6 (58 mg, 0.12 mmol) with 1 M 2-methoxyphenyl magnesium bromide/THF (150 μL, 0.15 mmol) to 2032.

### Example 20A

### Compound 2031

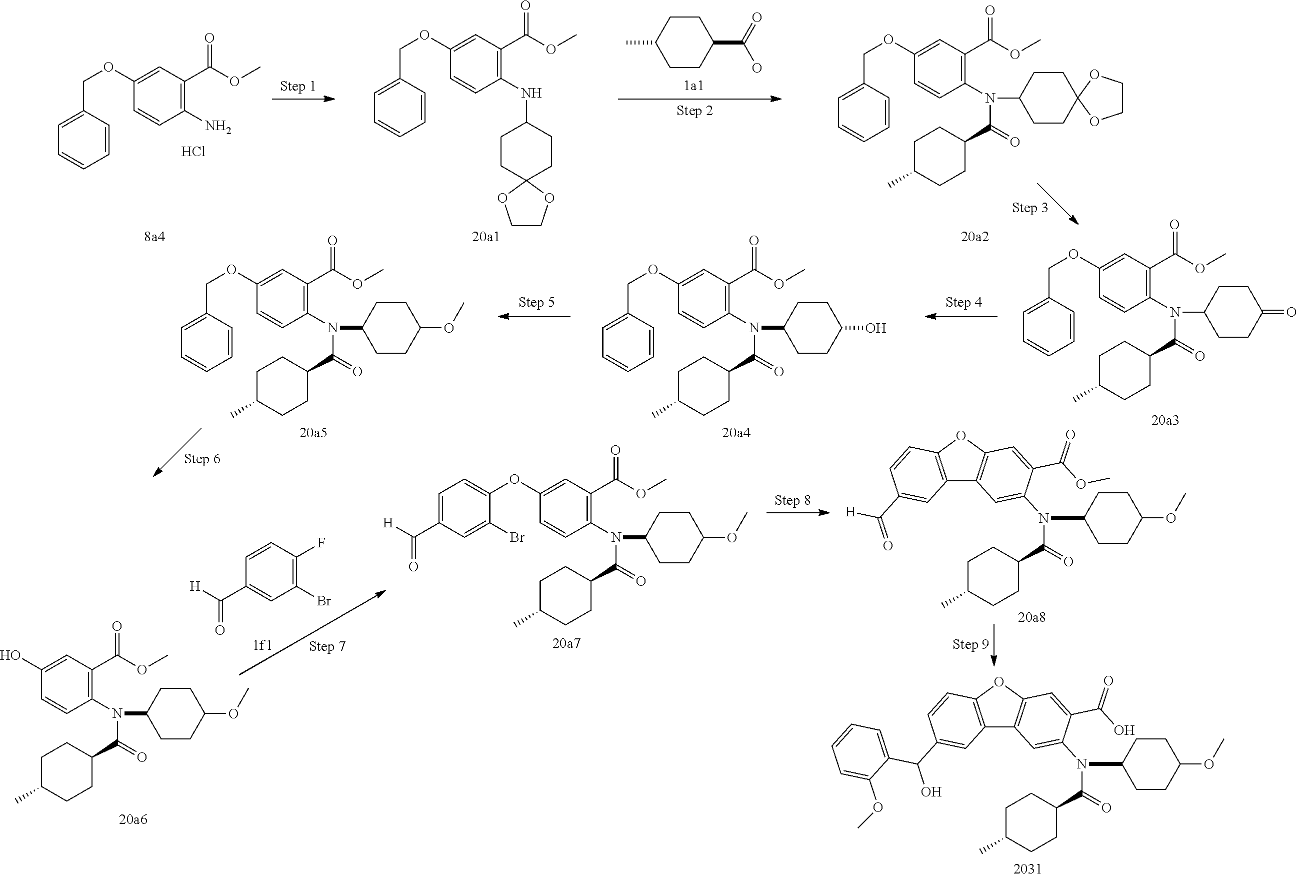

### Step 1:

Reference: WO 06/119646, pp. 60-61.

Dissolve 8a4 (12 g, 32 mmol) in water (100 mL) and add 1 M NaOH (aqueous) until the mixture is slightly basic. Extract with EtOAc. Wash the organic portion with water and brine, dry over Na₂SO₄, filter and evaporate. Dissolve the solids in anhydrous THF (20 mL) and add 1,4-cyclohexadione monoethylene ketal (5 g, 32 mmol) and dibutyltin dichloride (0.48 g, 1.58 mmol). Stir at RT for about 10 min. Add phenyl silane (4.30 mL, 34.7 mmol) and stir for about 3 days. Evaporate the volatiles and dissolve the residue in EtOAc. Wash with saturated NaHCO₃ (aqueous), and brine, dry over Na₂SO₄, filter and concentrate to give crude 20a1.

### Step 2:

Using the protocol described in Example 19A, Step 3, 20a1 is converted to 20a2.

### Step 3:

Stir a mixture of 20a2, toluene (40 mL), TFA (40 mL) and water (1.1 mL) for about 2 h. Evaporate the volatiles and dilute with EtOAc. Wash with saturated NaHCO₃ (aqueous) and brine, dry over Na₂SO₄, filter and concentrate to give crude 20a3.

### Step 4:

Using the protocol described in Example 4A, Step 1, 20a3 is converted to 20a4.

### Step 5:

Using the protocol described in Example 19A, Step 2, 20a4 is converted to 20a5.

### Step 6:

Using the protocol described in Example 19A, Step 4, 20a5 is converted to 20a6.

### Step 7:

Using the protocol described in Example 1C, Step 1, combine 20a6 (153 mg, 0.38 mmol), and 1f1 (85 mg, 0.42 mmol) in the presence of cesium carbonate (248 mg, 0.76 mmol) to give 20a7.

### Step 8:

Using the protocol described in Example 11A, Step 2, 20a7 (185 mg, 0.31 mmol) is converted to 2008.

### Step 9:

Using the protocol described for the production of 1180, convert 20a8 (58 mg, 0.12 mmol) with 1 M 2-methoxyphenyl magnesium bromide/THF (150 μL, 0.15 mmol) to 2031.

### Example 21A

### Cell-Based Luciferase Reporter HCV RNA Replication Assay

Representative compounds of the invention are tested for activity as inhibitors of hepatitis C virus RNA replication in cells expressing a stable subgenomic HCV replicon, using the assay described in WO 2005/025501, herein incorporated by reference.

### Tables of Compounds

The following tables list compounds representative of the invention. Representative compounds listed in Tables 1 to 3 below are tested in the assay of Example 21A and are found to have EC₅₀ values below 40 μM.

Retention times (t_{R}) for each compound are measured using the standard analytical HPLC conditions described in the Examples. As is well known to one skilled in the art, retention time values are sensitive to the specific measurement conditions. Therefore, even if identical conditions of solvent, flow rate, linear gradient, and the like are used, the retention time values may vary when measured, for example, on different HPLC instruments. Even when measured on the same instrument, the values may vary when measured, for example, using different individual HPLC columns, or, when measured on the same instrument and the same individual column, the values may vary, for example, between individual measurements taken on different occasions. The synthetic method used to generate each compound in Tables 1 to 3 is identified in the table. A person skilled in the art will recognize that obvious modifications to the synthetic methods may be required to generate each of the specific compounds listed in Tables 1 to 3.

TABLE 1

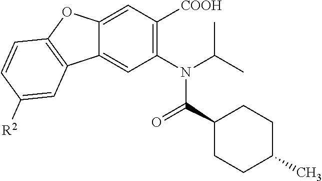

| | | t_{R} | MS | Synthetic |
|---|---|---|---|---|
| Cpd | R² | (min) | (M + H)⁺ | Method |
| 1001 | 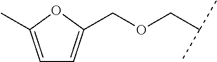 | 7.6 | 518.2 | 4C |
| 1002 | 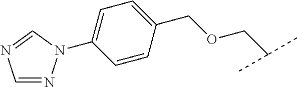 | 7.0 | 581.1 | 5A |
| 1003 | 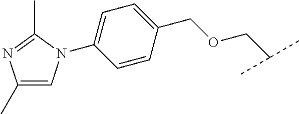 | 5.5 | 608.2 | 5A |
| 1004 | H | 6.7 | 394.2 | 1A |
| 1005 | SO₃H | 3.9 | 474.1 | 1B |
| 1006 | NO₂ | 6.5 | 439.2 | 1D |
| 1007 | C(═O)OH | 5.5 | 438.2 | 1E |
| 1008 | 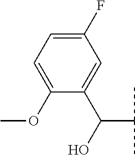 | 7.3 | 548.3 | 6A |
| 1009 | 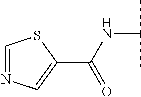 | 5.8 | 520.1 | 1K |
| 1010 | 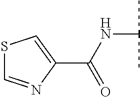 | 6.1 | 520.1 | 1K |
| 1011 | 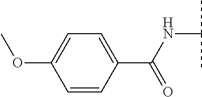 | 6.4 | 543.2 | 1K |
| 1012 | NH₂ | 3.8 | 409.2 | 1C |
| 1013 | 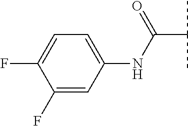 | 7.0 | 549.2 | 3A |
| 1014 | 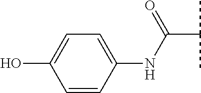 | 5.7 | 529.2 | 3A |
| 1015 | 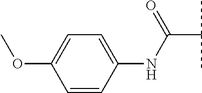 | 6.5 | 543.2 | 3A |
| 1016 | 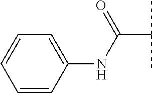 | 6.6 | 513.2 | 3A |
| 1017 | 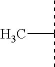 | 6.9 | 408.3 | 14A |
| 1018 | 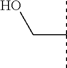 | 5.2 | 424.1 | 1F |
| 1019 | 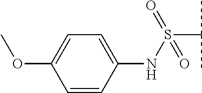 | 6.1 | 579.1 | 2A |
| 1020 | 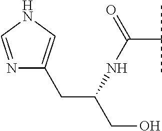 | 4.1 | 561.2 | 3A |
| 1021 | 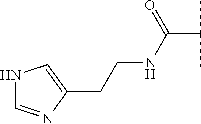 | 4.4 | 531.2 | 3A |
| 1022 | 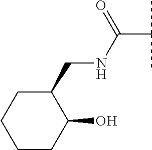 | 5.8 | 549.3 | 3A |
| 1023 | 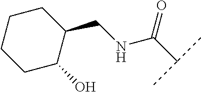 | 5.6 | 549.3 | 3A |
| 1024 | 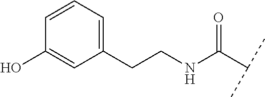 | 5.6 | 557.2 | 3A |
| 1025 | 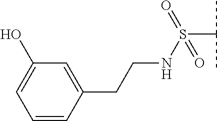 | 5.7 | 593.2 | 2A |
| 1026 | 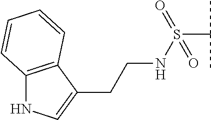 | 6.1 | 616.2 | 2A |
| 1027 | 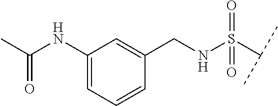 | 5.4 | 620.2 | 2A |
| 1028 | 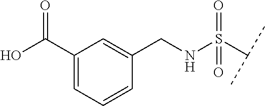 | 5.5 | 607.2 | 2A |
| 1029 | 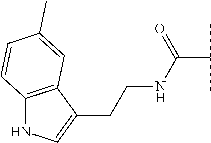 | 6.4 | 594.3 | 3A |
| 1030 | 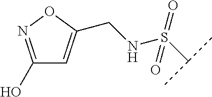 | 5.1 | 570.1 | 2A |
| 1031 | 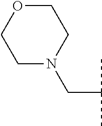 | 4.2 | 493.3 | 1L |
| 1032 | 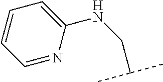 | 4.6 | 500.3 | 1L |
| 1033 | 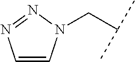 | 5.3 | 475.2 | 4A |
| 1034 | 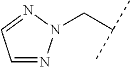 | 5.8 | 475.2 | 4A |
| 1035 | 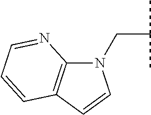 | 5.7 | 524.2 | 4A |
| 1036 | 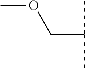 | 6.2 | 438.3 | 4B |
| 1037 | 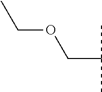 | 6.5 | 451.2 | 4B |
| 1038 | 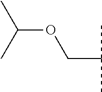 | 6.7 | 466.3 | 4B |
| 1039 | 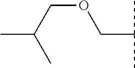 | 7.2 | 480.3 | 4B |
| 1040 | 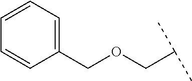 | 7.2 | 514.3 | 4C |
| 1041 | 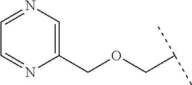 | 6.0 | 516.3 | 4C |
| 1042 | 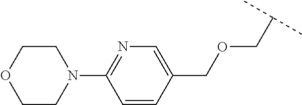 | 5.2 | 600.3 | 4C |
| 1043 | 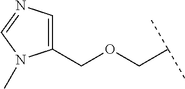 | 4.9 | 518.3 | 4C |
| 1044 | 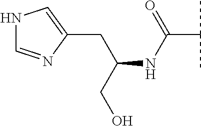 | 4.1 | 561.3 | 3A |
| 1045 | 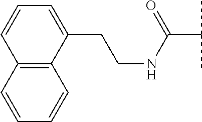 | 6.8 | 591.3 | 3A |
| 1046 | 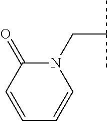 | 5.3 | 501.3 | 4A |
| 1047 | 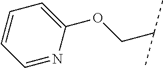 | 6.2 | 501.3 | 4A |
| 1048 | 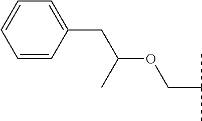 | 9.1 | 542.3 | 4C |
| 1049 | 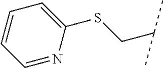 | 7.5 | 517.2 | 4A |
| 1050 | 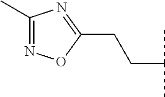 | 7.3 | 504.3 | 1N |
| 1051 | 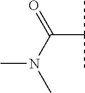 | 6.0 | 465.3 | 3A |
| 1052 | 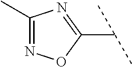 | 6.3 | 476.2 | 3B |
| 1053 | 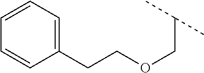 | 7.2 | 528.3 | 4C |
| 1054 | 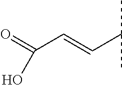 | 5.6 | 464.2 | 1G |
| 1055 | 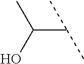 | 5.3 | 438.2 | 1G |
| 1056 | 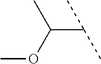 | 6.2 | 452.3 | 1G |
| 1057 | 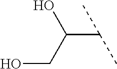 | 4.4 | 454.2 | 1H |
| 1058 | 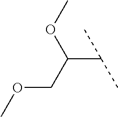 | 5.8 | 482.3 | 1H |
| 1059 | 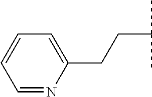 | 4.8 | 499.3 | 1L |
| 1060 | 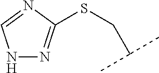 | 5.6 | 507.2 | 4A |
| 1061 | 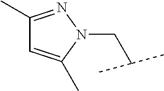 | 6.0 | 502.3 | 4A |
| 1062 | 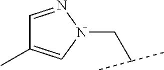 | 6.5 | 488.2 | 4A |
| 1063 | 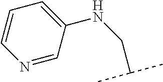 | 4.4 | 500.2 | 4A |
| 1064 | 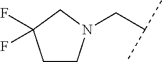 | 4.4 | 513.2 | 4A |
| 1065 | 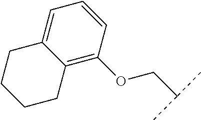 | 8.7 | 554.3 | 4A |
| 1066 | 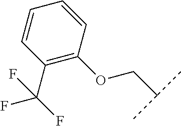 | 7.9 | 568.2 | 4A |
| 1067 | 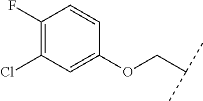 | 8.1 | 552.2 | 4A |
| 1068 | 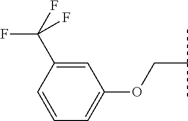 | 8.1 | 568.2 | 4A |
| 1069 | 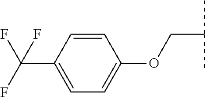 | 8.1 | 568.2 | 4A |
| 1070 | 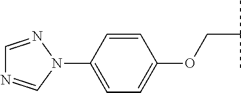 | 6.9 | 567.3 | 4A |
| 1071 | 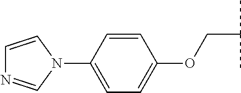 | 5.2 | 566.3 | 4A |
| 1072 | 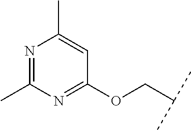 | 4.9 | 530.3 | 4A |
| 1073 | 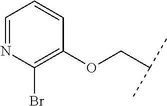 | 7.2 | 579.2 | 4A |
| 1074 | 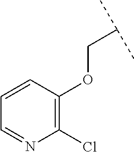 | 7.1 | 535.2 | 4A |
| 1075 | 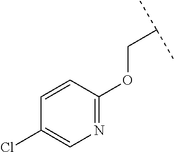 | 8.1 | 535.2 | 4A |
| 1076 | 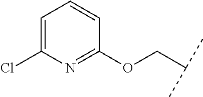 | 8.0 | 535.2 | 4A |
| 1077 | 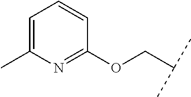 | 6.8 | 513.3 | 4A |
| 1078 | 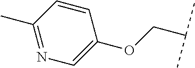 | 4.6 | 515.3 | 4A |
| 1079 | 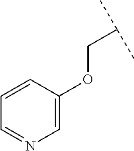 | 4.7 | 501.2 | 4A |
| 1080 | 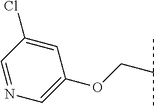 | 7.3 | 535.2 | 4A |
| 1081 | 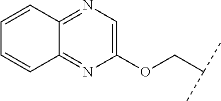 | 7.9 | 552.3 | 4A |
| 1082 | 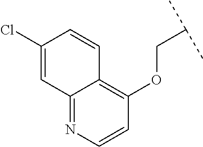 | 6.2 | 585.3 | 4A |
| 1083 | 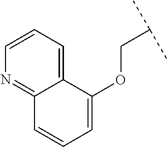 | 5.3 | 551.3 | 4A |
| 1084 | 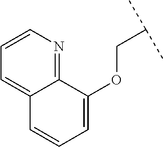 | 4.9 | 551.3 | 4A |
| 1085 | 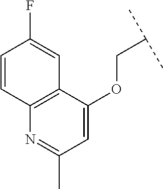 | 5.3 | 583.3 | 4A |
| 1086 | 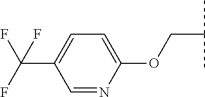 | 8.1 | 569.2 | 4A |
| 1087 | 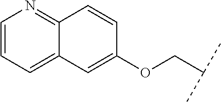 | 5.2 | 551.3 | 4A |
| 1088 | 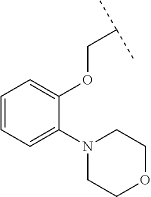 | 7.1 | 585.3 | 4A |
| 1089 | 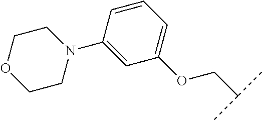 | 7.2 | 585.3 | 4A |
| 1090 | 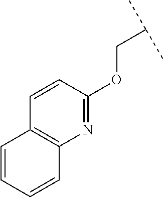 | 7.7 | 551.3 | 4A |
| 1091 | 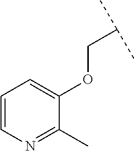 | 4.6 | 515.3 | 4A |
| 1092 | 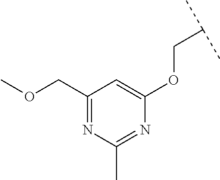 | 6.0 | 560.3 | 4A |
| 1093 | 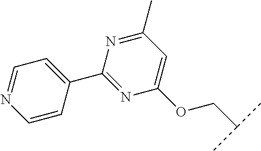 | 5.7 | 593.3 | 4A |
| 1094 | 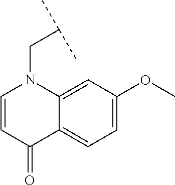 | 5.3 | 581.3 | 4A |
| 1095 | 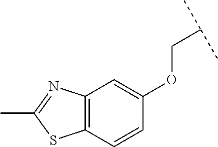 | 7.6 | 571.2 | 4A |
| 1096 | 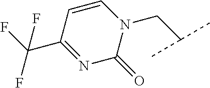 | 6.0 | 570.2 | 4A |
| 1097 | 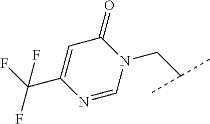 | 6.7 | 570.2 | 4A |
| 1098 | 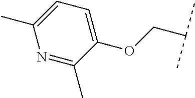 | 4.7 | 529.3 | 4A |
| 1099 | 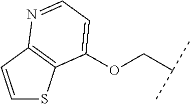 | 4.9 | 557.2 | 4A |
| 1100 | 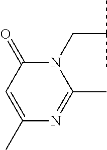 | 4.9 | 530.3 | 4A |
| 1101 | 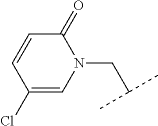 | 6.2 | 535.2 | 4A |
| 1102 | 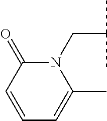 | 5.8 | 515.3 | 4A |
| 1103 | 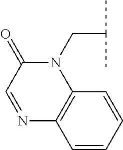 | 6.5 | 552.3 | 4A |
| 1104 | 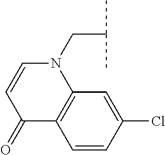 | 5.4 | 585.2 | 4A |
| 1105 | 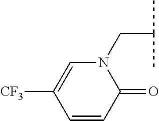 | 6.7 | 569.2 | 4A |
| 1106 | 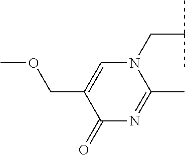 | 5.3 | 560.3 | 4A |
| 1107 | 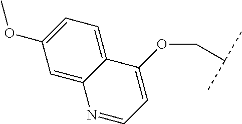 | 5.5 | 581.3 | 4A |
| 1108 | 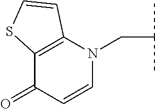 | 5.1 | 557.2 | 4A |
| 1109 | 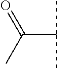 | 5.8 | 436.2 | 1M |
| 1110 | 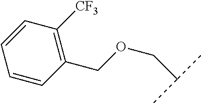 | 8.2 | 582.2 | 4C |
| 1111 | 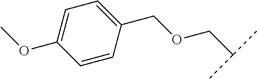 | 7.7 | 544.2 | 4C |
| 1112 | 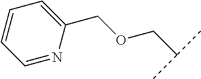 | 4.7 | 515.2 | 4C |
| 1113 | 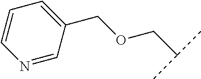 | 4.6 | 515.2 | 4C |
| 1114 | 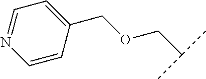 | 4.7 | 515.2 | 4C |
| 1115 | 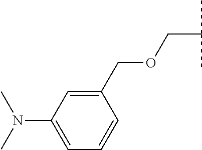 | 5.6 | 557.2 | 4C |
| 1116 | 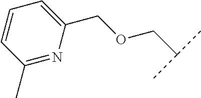 | 4.6 | 529.2 | 4C |
| 1117 | 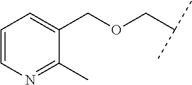 | 4.7 | 529.2 | 4C |
| 1118 | 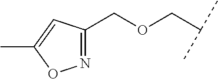 | 7.0 | 519.2 | 4C |
| 1119 | 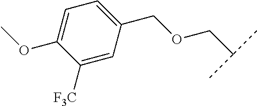 | 8.0 | 612.2 | 4C |
| 1120 | 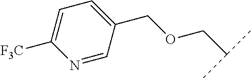 | 7.5 | 583.2 | 4C |
| 1121 | 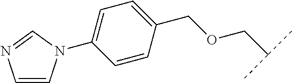 | 5.3 | 580.2 | 4C |
| 1122 | 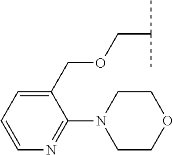 | 5.2 | 600.2 | 4C |
| 1123 | 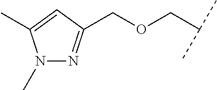 | 6.4 | 532.2 | 4C |
| 1124 | 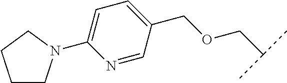 | 5.1 | 584.3 | 4C |
| 1125 | 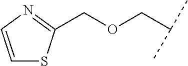 | 6.8 | 521.1 | 4C |
| 1126 | 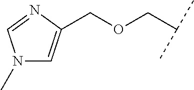 | 4.4 | 518.2 | 4C |
| 1127 | 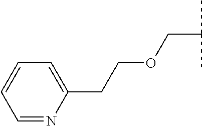 | 4.6 | 529.2 | 4C |
| 1128 | 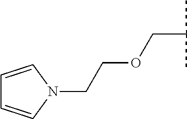 | 7.4 | 517.2 | 4C |
| 1129 | 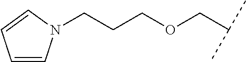 | 7.6 | 531.2 | 4C |
| 1130 | 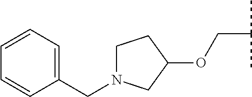 | 5.2 | 583.3 | 4C |
| 1131 | 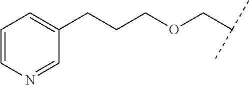 | 4.9 | 543.2 | 4C |
| 1132 | 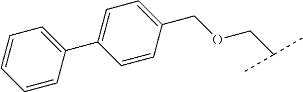 | 8.4 | 590.2 | 4C |
| 1133 | 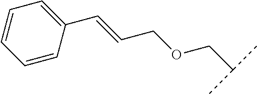 | 8.1 | 540.2 | 4C |
| 1134 | 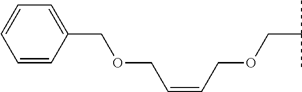 | 7.9 | 584.2 | 4C |
| 1135 | 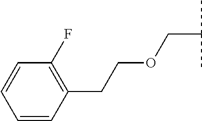 | 8.0 | 546.2 | 4C |
| 1136 | 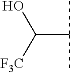 | 5.6 | 492.2 | 1I |
| 1137 | 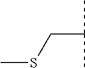 | 6.4 | 454.2 | 5B |
| 1138 | 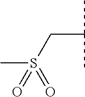 | 4.9 | 486.1 | 5B |
| 1139 | 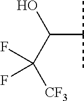 | 6.1 | 542.1 | 1I |
| 1140 | 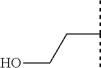 | 6.1 | 438.1 | 1H |
| 1141 | 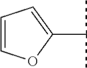 | 8.4 | 460.1 | 1J |
| 1142 | 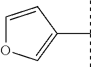 | 8.1 | 460.1 | 1J |
| 1143 | 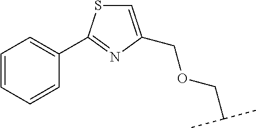 | 8.0 | 597.1 | 4C |
| 1144 | 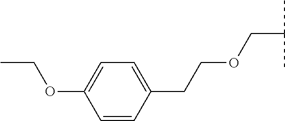 | 8.1 | 572.2 | 4C |
| 1145 | 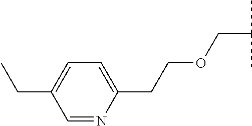 | 5.0 | 557.2 | 4C |
| 1146 | 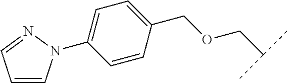 | 7.6 | 580.2 | 4C |
| 1147 | 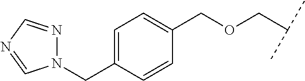 | 6.6 | 595.2 | 4C |
| 1148 | 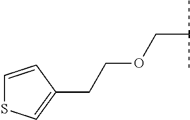 | 7.8 | 534.1 | 4C |
| 1149 | 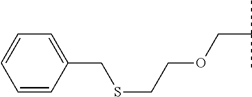 | 8.0 | 574.2 | 4C |
| 1150 | 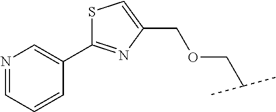 | 6.0 | 598.1 | 4C |
| 1151 | 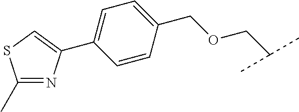 | 7.8 | 611.2 | 4C |
| 1152 | 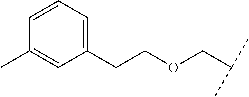 | 8.2 | 542.2 | 4C |
| 1153 | 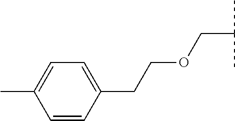 | 8.2 | 542.2 | 4C |
| 1154 | 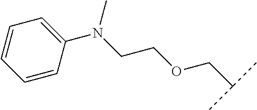 | 5.8 | 557.2 | 4C |
| 1155 | 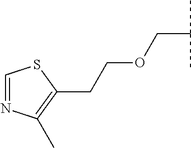 | 6.0 | 549.2 | 4C |
| 1156 | 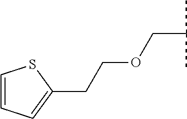 | 7.9 | 534.1 | 4C |
| 1157 | 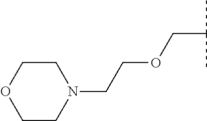 | 4.4 | 537.2 | 4C |
| 1158 | 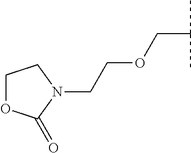 | 5.9 | 537.2 | 4C |
| 1159 | 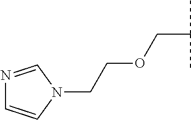 | 4.5 | 518.2 | 4C |
| 1160 | 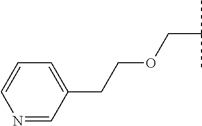 | 4.7 | 529.2 | 4C |
| 1161 | 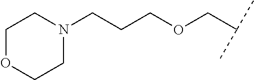 | 4.6 | 551.2 | 4C |
| 1162 | 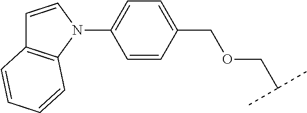 | 8.5 | 629.1 | 5A |
| 1163 | 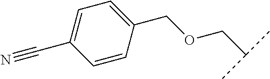 | 6.7 | 539.2 | 4C |
| 1164 | 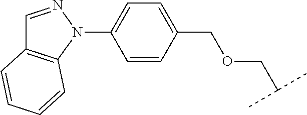 | 8.2 | 630.1 | 5A |
| 1165 | 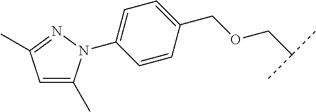 | 7.4 | 608.2 | 5A |
| 1166 | 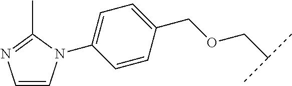 | 5.4 | 594.1 | 5A |
| 1167 | 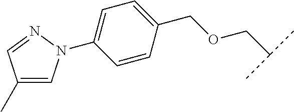 | 7.8 | 594.1 | 5A |
| 1168 | 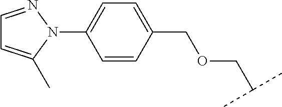 | 7.4 | 594.1 | 5A |
| 1169 | 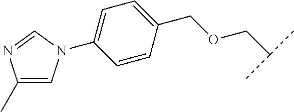 | 5.4 | 594.1 | 5A |
| 1170 | 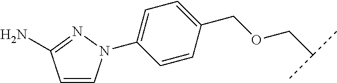 | 6.3 | 595.1 | 5A |
| 1171 | 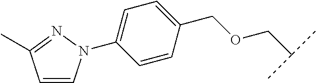 | 7.7 | 594.1 | 5A |
| 1172 | 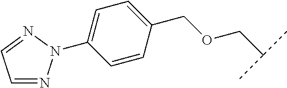 | 7.8 | 581.1 | 5A |
| 1173 | 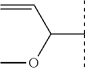 | 7.9 | 464.4 | 1O |
| 1174 | 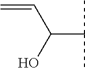 | 6.5 | 450.3 | 1O |
| 1175 | 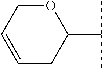 | 8.0 | 476.4 | 1P |
| 1176 | 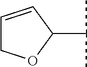 | 7.4 | 462.3 | 1O |
| 1177 | 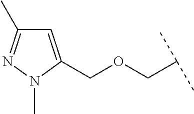 | 8.6 | 532.2 | 4C |
| 1178 | 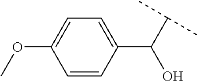 | 7.1 | 530.2 | 6A |
| 1179 | 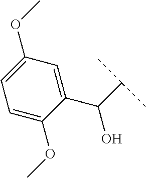 | 7.1 | 560.3 | 6A |
| 1180 | 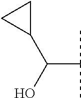 | 6.7 | 464.3 | 6A |
| 1181 | 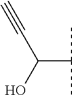 | 6.3 | 448.2 | 6A |
| 1182 | 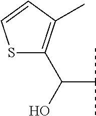 | 7.3 | 520.2 | 6A |
| 1183 | 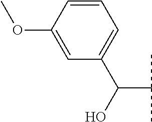 | 7.1 | 530.3 | 6A |
| 1184 | 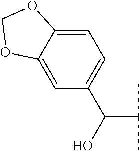 | 7.0 | 544.2 | 6A |
| 1185 | 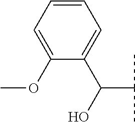 | 7.3 | 530.3 | 6A |
| 1186 | 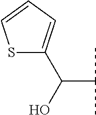 | 7.2 | 506.2 | 6A |
| 1187 | 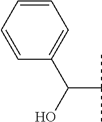 | 7.3 | 500.3 | 6A |
| 1188 | 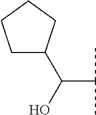 | 7.7 | 492.3 | 6A |
| 1189 | 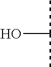 | 6.3 | 410.1 | 7A |
| 1190 | 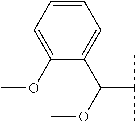 | 7.6 | 544.3 | 6A |

TABLE 2

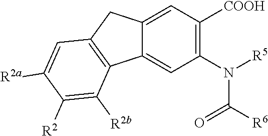

| | | | | | | t_{R} | MS | Syn |
|---|---|---|---|---|---|---|---|---|
| Cpd | R² | R^{2a} | R^{2b} | R⁵ | R⁶ | (min) | (M + H)⁺ | Meth |
| 2001 | 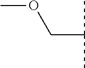 | F | H | 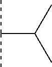 | 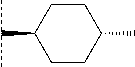 | 7.6 | 456.2 | 10A |
| 2002 | H | H | 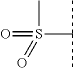 | 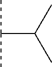 | 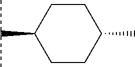 | 5.6 | 472.1 | 1Q |
| 2003 | 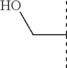 | H | H | 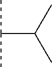 | 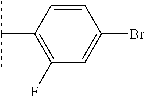 | 4.7 | 502.0 | 8A |
| 2004 | 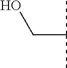 | H | H | 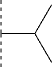 | 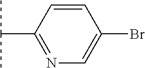 | 4.3 | 485.0 | 8A |
| 2005 | 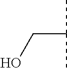 | H | H | 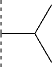 | 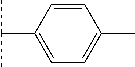 | 5.2 | 418.1 | 8A |
| 2006 | 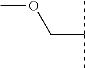 | H | H | 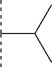 | 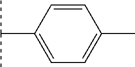 | 6.6 | 432.1 | 8A |
| 2007 | 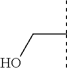 | 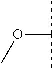 | H | 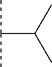 | 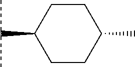 | 6.1 | 454.2 | 10A |
| 2008 | 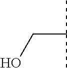 | 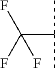 | H | 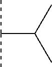 | 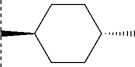 | 7.2 | 492.1 | 10A |
| 2009 | 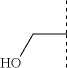 | 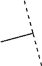 | H | 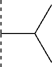 | 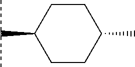 | 6.4 | 438.2 | 10A |
| 2010 | 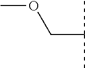 | 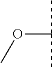 | H | 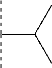 | 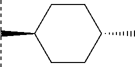 | 7.5 | 468.2 | 10A |
| 2011 | 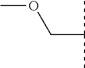 | 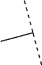 | H | 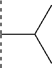 | 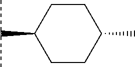 | 7.8 | 452.2 | 10A |
| 2012 | 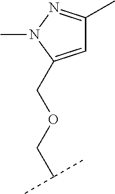 | 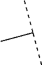 | H | 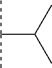 | 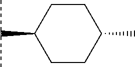 | 7.0 | 546.2 | 10A |
| 2013 | 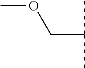 | F | F | 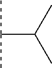 | 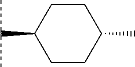 | 7.7 | 474.1 | 10A |
| 2014 | H | 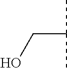 | H | 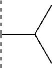 | 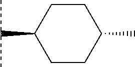 | 5.8 | 424.2 | 11A |
| 2015 | H | 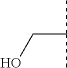 | H | 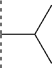 | 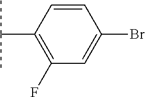 | 5.7 | 502.0 | 11A |
| 2016 | H | H | H | 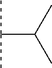 | 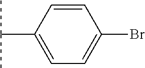 | 6.7 | 452 | 9A |
| 2017 | H | H | H | 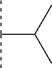 | 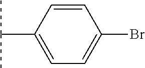 | 6.3 | 388.1 | 9A |
| 2018 | H | H | H | 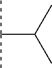 | 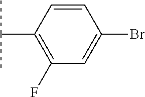 | 6.9 | 470 | 9A |
| 2019 | H | H | H | 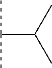 | 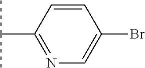 | 6.3 | 453 | 9A |
| 2020 | H | 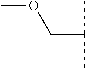 | H | 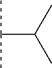 | 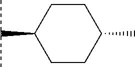 | 7.4 | 438.2 | 11A |
| 2021 | H | 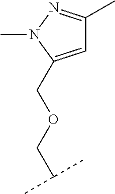 | H | 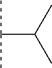 | 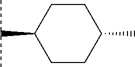 | 6.6 | 532.2 | 11A |
| 2022 | 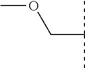 | H | H | 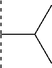 | 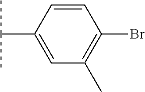 | 6.8 | 510 | 8A |
| 2023 | 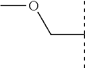 | H | H | 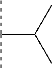 | 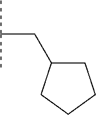 | 6.6 | 424.2 | 8A |
| 2024 | 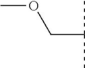 | H | H | 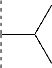 | 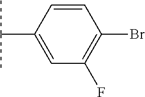 | 6.7 | 514 | 8A |
| 2025 | 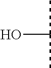 | H | H | 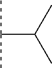 | 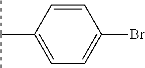 | 5.4 | 404.2 | 7B |
| 2026 | 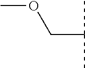 | H | H | 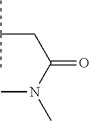 | 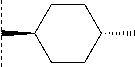 | 6.6 | 481.2 | 12A |
| 2027 | 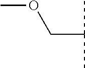 | H | H | 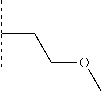 | 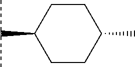 | 7.0 | 454.2 | 12A |
| 2028 | 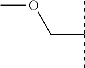 | H | H | 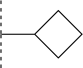 | 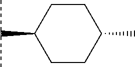 | 7.6 | 450.2 | 13A |
| 2029 | 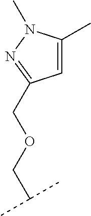 | H | H | 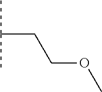 | 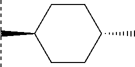 | 6.6 | 548.2 | 12A |
| 2030 | 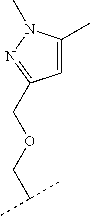 | H | H | 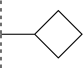 | 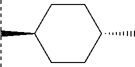 | 7.1 | 544.2 | 13A |
| 2031 | 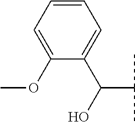 | H | H | 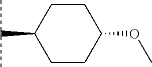 | 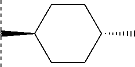 | 6.7 | 600.3 | 20A |
| 2032 | 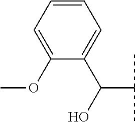 | H | H | 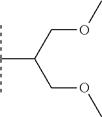 | 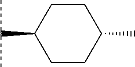 | 6.7 | 590.3 | 19A |
| 2033 | 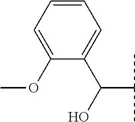 | H | H | 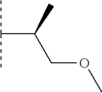 | 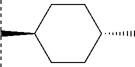 | 6.7 | 560.3 | 18A |

TABLE 3

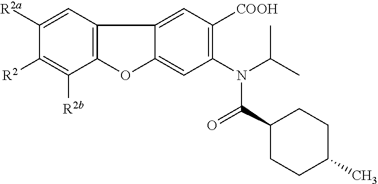

| | | | | t_{R} | MS | Syn |
|---|---|---|---|---|---|---|
| Cpd | R² | R^{2a} | R^{2b} | (min) | (M + H)⁺ | Met |
| 3001 | 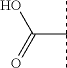 | H | H | 6.0 | 438.1 | 16A |
| 3002 | H | H | H | 6.7 | 394.2 | 15A |
| 3003 | H | H | 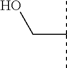 | 4.9 | 424.2 | 17A |
| 3004 | H | H | 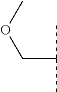 | 5.7 | 438.2 | 17A |

Each of the references, including all patents, patent applications and publications, listed in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A compound of formula (I): 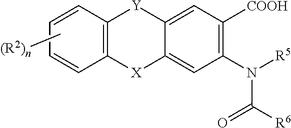
wherein:
either X is absent and Y is O; or
Y is absent and X is O;
n is 0 to 4;
R² is selected from:
a) halo, cyano, nitro or SO₃H;
b) R⁷, —C(═O)—R⁷, —C(═O)—O—R⁷, —O—R⁷, —S—R⁷, —SO—R⁷, —SO₂—R⁷, —(C₁₋₆)alkylene-R⁷, —(C₁₋₆)alkylene-C(═O)—R⁷, —(C₁₋₆)alkylene-C(═O)—O—R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl, —N((C₁₋₄)alkyl)₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
wherein each of the aryl and Het is optionally substituted with 1 to 3 substituents each independently selected from:
i) halo, cyano, oxo, thioxo, imino, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
ii) (C₁₋₆)alkyl optionally substituted with —OH, —O—(C₁₋₆)haloalkyl, or —O—(C₁₋₆)alkyl; and
iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
c) —N(R⁸)R⁹, —C(═O)—N(R⁸)R⁹, —O—C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹, —(C₁₋₆)alkylene-O—C(═O)—N(R⁸)R⁹, or —(C₁₋₆)alkylene-SO₂—N(R⁸)R⁹; wherein the (C₁₋₆)alkylene is optionally substituted with 1 or 2 substituents each independently selected from —OH, —(C₁₋₆)alkyl, halo, (C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
R⁸ is in each instance independently selected from H, (C₁₋₆)alkyl and (C₃₋₇)cycloalkyl; and
R⁹ is in each instance independently selected from R⁷, —O—(C₁₋₆)alkyl, —(C₁₋₆)alkylene-R⁷, —SO₂—R⁷, —C(═O)—R⁷, —C(═O)OR⁷ and —C(═O)N(R⁸)R⁷;
wherein R⁷ and R⁸ are as defined above;
or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
wherein the heterocycle is optionally substituted with 1 to 3 substituents each independently selected from (C₁₋₆)alkyl, (C₁₋₆)haloalkyl, halo, oxo, —OH, SH, —O(C₁₋₆)alkyl, —S(C₁₋₆)alkyl, (C₃₋₇)cycloalkyl, —NH₂,
—NH(C₁₋₆)alkyl, —N((C₁₋₆)alkyl)₂, —NH(C₃₋₇)cycloalkyl,
—N((C₁₋₄)alkyl)(C₃₋₇)cycloalkyl, —C(═O)(C₁₋₆)alkyl and —NHC(═O)—(C₁₋₆)alkyl;
R⁵ is H, (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het; each being optionally substituted with 1 to 4 substituents each independently selected from (C₁₋₆)alkyl, (C₁₋₆)haloalkyl, (C₃₋₇)cycloalkyl, Het, —OH, —COOH, —C(═O)—(C₁₋₆)alkyl, —C(═O)—O—(C₁₋₆)alkyl, —SO₂(C₁₋₆)alkyl, —C(═O)—N(R⁵¹)R⁵² and —O—R⁵³;
wherein R⁵³ is (C₁₋₆)alkyl, (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het, said aryl and Het being optionally substituted with (C₁₋₆)alkyl or —O—(C₁₋₆)alkyl;
wherein R⁵¹ is H, (C₁₋₆)alkyl or (C₃₋₇)cycloalkyl; and
R⁵² is H, (C₁₋₆)alkyl, (C₃₋₇)cycloalkyl, aryl, Het, —(C₁₋₃)alkyl-aryl or —(C₁₋₃)alkyl-Het;
wherein each of the (C₁₋₆)alkyl, (C₃₋₇)cycloalkyl, aryl, Het, —(C₁₋₃)alkyl-aryl and —(C₁₋₃)alkyl-Het are optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
wherein the (C₁₋₆)alkyl is optionally substituted with OH;
or R⁵¹ and R⁵², together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
wherein the heterocycle is optionally substituted with 1 to 3 substituents each independently selected from (C₁₋₆)alkyl, (C₁₋₆)haloalkyl, halo, oxo, —OH, —O(C₁₋₆)alkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
R⁶ is (C₃₋₇)cycloalkyl, —(C₁₋₆)alkyl-(C₃₋₇)cycloalkyl, aryl, —(C₁₋₆)alkyl-aryl, Het or —(C₁₋₆)alkyl-Het; being optionally substituted with 1 to 5 substituents each independently selected from halo, (C₁₋₆)alkyl, (C₁₋₆)haloalkyl, (C₃₋₇)cycloalkyl, —OH, —SH, —O—(C₁₋₄)alkyl, —S—(C₁₋₄)alkyl and —N(R⁸)R⁹; wherein R⁸ and R⁹ are as defined above; and
Het is a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S;
wherein each N heteroatom may, independently and where possible, exist in an oxidized state such that it is further bonded to an oxygen atom to form an N-oxide group and wherein each S heteroatom may, independently and where possible, exist in an oxidized state such that it is further bonded to one or two oxygen atoms to form the groups SO or SO₂;
or a salt or ester thereof.

2. The compound according to claim 1, of the formula: 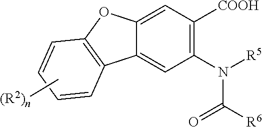
wherein R², n, R⁵ and R⁶ are as defined in claim 1, or a pharmaceutically acceptable salt or ester thereof.

3. The compound according to claim 1, of the formula: 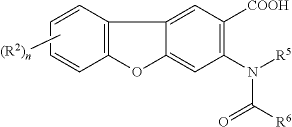
wherein R², n, R⁵ and R⁶ are as defined in claim 1, or a pharmaceutically acceptable salt or ester thereof.

4. The compound according to claim 1, wherein R² is selected from:
a) halo, nitro or SO₃H;
b) R⁷, C(═O)OH, C(═O)(C₁₋₆)alkyl, —O—R⁷, —S—R⁷, —SO—R⁷, —SO₂—R⁷, —(C₁₋₆)alkylene-R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl and (C₁₋₆)alkylene are optionally substituted with 1 or 2 substituents each independently selected from —OH, —(C₁₋₆)alkyl optionally substituted with —O—(C₁₋₆)alkyl, halo, (C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl, —N((C₁₋₄)alkyl)₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
wherein each of the aryl and Het is optionally substituted with 1 to 3 substituents each independently selected from:
i) halo, cyano, oxo, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
ii) (C₁₋₆)alkyl optionally substituted with —OH, —O—(C₁₋₆)haloalkyl, or —O—(C₁₋₆)alkyl; and
iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
c) —N(R⁸)R⁹, —C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹ or —(C₁₋₆)alkylene-SO₂—N(R⁸)R⁹; wherein the (C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl and —N((C₁₋₄)alkyl)₂;
R⁸ is in each instance independently selected from H and (C₁₋₆)alkyl; and
R⁹ is in each instance independently selected from R⁷, —O—(C₁₋₆)alkyl, —(C₁₋₆)alkylene-R⁷, —SO₂—R⁷, —C(═O)—R⁷; wherein R⁷ is as defined above, or a pharmaceutically acceptable salt or ester thereof.

5. The compound according to claim 4, wherein R² is selected from:
a) halo, nitro or SO₃H;
b) R⁷, C(═O)OH, C(═O)(C₁₋₆)alkyl, —O—R⁷, —SO₂—R⁷, —(C₁₋₆)alkylene-R⁷, —(C₁₋₆)alkylene-O—R⁷, —(C₁₋₆)alkylene-S—R⁷ or —(C₁₋₆)alkylene-SO₂—R⁷;
wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, —(C₁₋₆)alkyl optionally substituted with —O—(C₁₋₆)alkyl, halo, (C₁₋₆)haloalkyl, (C₃₋₇)cycloalkyl, —O—(C₁₋₆)alkyl, COOH, —NH₂, —N((C₁₋₄)alkyl)(aryl), aryl, —(C₁₋₆)alkyl-aryl, —O—(C₁₋₆)alkyl-aryl, —S—(C₁₋₆)alkyl-aryl, Het, —(C₁₋₆)alkyl-Het, —O—(C₁₋₆)alkyl-Het; and
wherein each of the aryl and Het is optionally substituted with 1 to 3 substituents each independently selected from:
i) halo, cyano, oxo, —OH, —O—(C₁₋₆)alkyl, —O—(C₁₋₆)haloalkyl, (C₃₋₇)cycloalkyl, (C₁₋₆)haloalkyl, —C(═O)—(C₁₋₆)alkyl, COOH, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂ or —NH—C(═O)(C₁₋₄)alkyl;
ii) (C₁₋₆)alkyl optionally substituted with —OH, —O—(C₁₋₆)haloalkyl, or —O—(C₁₋₆)alkyl; and
iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
c) —N(R⁸)R⁹, —C(═O)—N(R⁸)R⁹, —SO₂—N(R⁸)R⁹, —(C₁₋₆)alkylene-N(R⁸)R⁹, —(C₁₋₆)alkylene-C(═O)—N(R⁸)R⁹ or —(C₁₋₆)alkylene-SO₂—N(R⁸)R⁹; wherein the (C₁₋₆)alkylene is optionally substituted with 1 or 2 substituents each independently selected from —OH, —(C₁₋₆)alkyl, halo, —(C₁₋₆)haloalkyl, —O—(C₁₋₆)alkyl;
R⁸ is in each instance independently selected from H and (C₁₋₆)alkyl; and
R⁹ is in each instance independently selected from R⁷, —O— (C₁₋₆)alkyl, —(C₁₋₆)alkylene-R⁷, —C(═O)—R⁷; wherein R⁷ is as defined above; or a pharmaceutically acceptable salt or ester thereof.

6. The compound according to claim 5, wherein R² is selected from:
a) halo, nitro or SO₃H;
b) R⁷, OH, C(═O)OH, C(═O)(C₁₋₆)alkyl, —SO₂—R⁷, —(C₁₋₆)alkylene-R⁷, —(C₁₋₆)alkylene-O—R⁷, —(C₁₋₆)alkylene-S—R⁷ or —(C₁₋₆)alkylene-SO₂—R⁷;
wherein R⁷ is in each instance independently selected from H, (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, aryl and Het;
wherein the (C₁₋₆)alkyl, (C₂₋₆)alkenyl, (C₂₋₆)alkynyl, (C₁₋₆)haloalkyl, (C₃₋₇)cycloalkyl, —(C₁₋₆)alkyl-(C₃₋₇)cycloalkyl, and (C₁₋₆)alkylene are optionally substituted with 1 or 2 substituents each independently selected from —OH, halo, —(C₁₋₆)haloalkyl, —O—(C₁₋₆)alkyl, COOH, —N((C₁₋₄)alkyl)(aryl), aryl, —(C₁₋₆)alkyl-aryl, —O—(C₁₋₆)alkyl-aryl,
—S—(C₁₋₆)alkyl-aryl, Het, —(C₁₋₆)alkyl-Het, —O—(C₁₋₆)alkyl-Het; and
wherein each of the aryl and Het is optionally substituted with 1 to 3 substituents each independently selected from:
i) halo, cyano, oxo, —OH, —O—(C₁₋₆)alkyl, (C₁₋₆)haloalkyl, —NH₂, —N((C₁₋₄)alkyl)₂ or —NH—C(═O)(C₁₋₄)alkyl;
ii) (C₁₋₆)alkyl optionally substituted with —O—(C₁₋₆)alkyl; and
iii) aryl, —(C₁₋₆)alkyl-aryl, Het or —(C₁₋₆)alkyl-Het, wherein each of the aryl and Het is optionally substituted with halo, (C₁₋₆)alkyl or NH₂; and
c) —N(R⁸)R⁹, —C(═O)—N(R⁸)R⁹, —SO₂—N(R⁸)R⁹ or —(C₁₋₆)alkylene-N(R⁸)R⁹;
R⁸ is H; and
R⁹ is in each instance independently selected from R⁷, —(C₁₋₆)alkylene-R⁷ or —C(═O)—R⁷, wherein R⁷ is as defined above; or a pharmaceutically acceptable salt or ester thereof.

7. The compound according to claim 1, wherein R⁵ is (C₁₋₆)alkyl, (C₃₋₇)cycloalkyl, —(C₁₋₆)alkyl-(C₃₋₇)cycloalkyl; each being optionally substituted with 1 to 2 substituents each independently selected from (C₁₋₆)alkyl, —OH, —C(═O)—(C₁₋₆)alkyl, —C(═O)—O—(C₁₋₆)alkyl, —C(═O)—N(R⁵¹)R⁵² and O—R⁵³; wherein R⁵³ is (C₁₋₆)alkyl, (C₃₋₇)cycloalkyl or —(C₁₋₆)alkyl-(C₃₋₇)cycloalkyl; R⁵¹ is H, (C₁₋₆)alkyl or (C₃₋₇)cycloalkyl; and R⁵² is H, (C₁₋₆)alkyl or
(C₃₋₇)cycloalkyl; or a pharmaceutically acceptable salt or ester thereof.

8. The compound according to claim 7, wherein R⁵ is (C₁₋₄)alkyl or (C₃₋₇)cycloalkyl; each being optionally substituted with 1 to 2 substituents each independently selected from (C₁₋₄)alkyl, —C(═O)—N(R⁵¹)R⁵² and
—O—(C₁₋₄)alkyl; R⁵¹ is (C₁₋₄)alkyl; and R⁵² is (C₁₋₄)alkyl; or a pharmaceutically acceptable salt or ester thereof.

9. The compound according to claim 1, wherein R⁶ is (C₅₋₆)cycloalkyl, —(C₁₋₃)alkyl-(C₅₋₆)cycloalkyl, phenyl or Het optionally substituted with 1 to 3 substituents each ndependently selected from halo, (C₁₋₄)alkyl and (C₁₋₄)haloalkyl; wherein Het is a 4- to 7-membered saturated, unsaturated or aromatic heterocycle having 1 to 3 nitrogen heteroatoms; or a pharmaceutically acceptable salt or ester thereof.

10. The compound according to claim 9, wherein R⁶ is phenyl, cyclohexyl, —CH₂-cyclopentyl or pyridine optionally substituted with 1 to 3 substituents each independently selected from halo, (C₁₋₄)alkyl and (C₁₋₄)haloalkyl; or a pharmaceutically acceptable salt or ester thereof.

11. The compound according to claim 10, wherein R⁶ is cyclohexyl or —CH₂-cyclopentyl, optionally substituted with 1 to 3 substituents each independently selected from halo, (C₁₋₄)alkyl and (C₁₋₄)haloalkyl; or a pharmaceutically acceptable salt or ester thereof.

12. (canceled)

13. A pharmaceutical composition comprising a therapeutically effective amount of a compound of formula (I) according to claim 1, or a pharmaceutically acceptable salt or ester thereof, and one or more pharmaceutically acceptable carriers.

14. The pharmaceutical composition according to claim 13 additionally comprising at least one other antiviral agent.

15. A method for the treatment of a hepatitis C viral infection in a mammal having or at risk of having the infection, said method comprising administering to said mammal a therapeutically effective amount of a compound of formula (I) according to claim 1, or a pharmaceutically acceptable salt or ester thereof.
